(19) United States
(12) Patent Application Publication
(10) Pub. No.: US 2024/0293464 A1
(43) Pub. Date: Sep. 5, 2024

(54) TISSUE REPAIR

(71) Applicant: Mohamed Hamza NOORDEEN, London (GB)

(72) Inventor: Mohamed Hamza NOORDEEN, London (GB)

(73) Assignee: REGENALL LIMITED, London (GB)

(21) Appl. No.: 18/287,554

(22) PCT Filed: Apr. 20, 2022
(86) PCT No.: PCT/GB2022/050994

§ 371 Date: Oct. 19, 2023

(30) Foreign Application Priority Data
Apr. 20, 2021 (GB) 2105646.0
May 28, 2021 (GB) 2107696.3
May 28, 2021 (GB) 2107701.1
May 28, 2021 (GB) 2107704.5
Oct. 1, 2021 (GB) 2114128.8
Oct. 1, 2021 (GB) 2114129.6
Oct. 1, 2021 (GB) 2114130.4

(51) Int. Cl. A61K 35/28; A61K 31/203; A61P 17/02; A61P 19/04

(52) U.S. Cl. CPC A61K 35/28; A61K 31/203; A61P 17/02; A61P 19/04

(57) ABSTRACT

Compounds, compositions, combined preparations, and multiple-dose formulations, for use in the treatment of tissue damage or injury, in particular, for example, spinal cord injury and tendon injury, are described. Methods of treatment of tissue damage or injury using the compounds, compositions, combined preparations, or multiple-dose formulations, are also described. Vitamin A. and optionally one or more regenerative cells, is used to inhibit scar tissue formation in a subject, which is a necessary prerequisite for successful tissue regeneration following tissue damage or injury. Vitamin A and compositions comprising vitamin A, for use in the treatment of pulmonary fibrosis, including pulmonary fibrosis caused by respiratory infection, are also described. Methods of treatment of pulmonary fibrosis using vitamin A and compositions are also described. Vitamin A. and compositions, combined preparations, and multiple-dose formulations comprising vitamin A, for use in the treatment of acute and chronic traumatic brain injury are also described, as well as methods of treatment of such injury using vitamin A. or the compositions, combined preparations, or multiple-dose formulations.

This invention relates to compounds, compositions, combined preparations, and multiple-dose formulations, for use in the treatment of tissue damage or injury, in particular, for example, spinal cord injury and tendon injury, and to methods of treatment of tissue damage or injury using the compounds, compositions, combined preparations, or multiple-dose formulations.

Tissue repair encompasses two separate processes: regeneration and replacement. Regeneration refers to a type of healing in which new growth completely restores portions of damaged tissue to their normal state. Replacement refers to a type of healing in which severely damaged or non-regenerable tissues are repaired by the laying down of connective tissue (or glial tissue in the central nervous system, for example the brain or spinal cord), a process commonly referred to as scarring. Whilst some types of tissue injury (such as minor paper cuts) can sometimes be healed in such a way that no permanent damage remains, most tissue repair consists of both regeneration and replacement. Tissue repair may restore some of the original structures of the damaged tissue (such as epithelial layers), but may also result in structural abnormalities that impair function (such as the scar formed in the healing of a myocardial infarction).

The extent to which regeneration and/or replacement occurs following injury depends, in part, on the type of tissue involved. Certain tissues of the body are more capable of cellular proliferation (and hence regeneration) than others. There are three types of tissues: continuously dividing tissues, quiescent tissues, and non-dividing tissues. Continuously dividing tissues are comprised of cells that are constantly proliferating in order to replace dead or sloughed-off cells. Examples of such tissues include epithelia (such as skin, gastrointestinal epithelium and salivary gland tissue) and hematopoietic tissues. These tissues contain pools of stem cells, which have enormous proliferative and self-renewing ability, and which give rise to more than one type of cell. Replicating asymmetrically, each stem cell gives rise to one daughter cell that differentiates and matures and another daughter cell that remains undifferentiated and capable of beginning another self-renewing cycle. Quiescent tissues are composed of cells that normally exist in a non-dividing state but may enter the cell cycle in response to certain stimuli, such as cell injury. Tissues falling into this category include parenchymal cells of the liver, kidney and pancreas, mesenchymal cells such as fibroblasts and smooth muscle cells, endothelial cells and lymphocytes. A few types of tissue are composed of cells that have left the cell cycle permanently, and are therefore unable to proliferate. These non-dividing tissues include cardiac and skeletal muscle. Tissue repair in these tissues always leaves permanent evidence of injury, such as a scar.

In adults, most cells, such as myocytes, adipocytes, skin cells and neurons, are in the non-dividing state, i.e. terminally-differentiated. Terminal differentiation is the process by which cells during the course of development become specialized, taking on specific structural, functional, and biochemical properties and roles. The brain is composed of both non-dividing and dividing cells. Specifically, differentiated neurons are in a post-mitotic state and cannot re-enter the cell cycle. Glial cells (e.g., astrocytes, oligodendrocytes, and microglia) are in either a proliferative or non-proliferative state, depending on their differentiation status and possible re-entry into the cell cycle.

Spinal cord injury (SCI) is a major cause of long-term physical impairment. It results in disruption of cord microstructure and is followed by limited neuronal regeneration and insufficient functional recovery in adult mammals. In some cases, nerve roots may be torn or avulsed from the spinal cord, leading to degeneration of nerve fibres, death of nerve cells and a breakdown of connections within the spinal cord. Root avulsion has been associated with an overall poor clinical outcome. Scar tissue formation in spinal cord injury is reviewed in O'Shea et al. Cell biology of spinal cord injury and repair, J. Clin. Invest. 2017, 127(9), 3259-3270. Briefly, after severe SCI, and in response to changes in the local microenvironment, astrocytes, the most abundant glial cells in the central nervous system (CNS) transform into reactive astrocytes and undergo dramatic morphological changes as well as massive variations in gene expression. Reactive astrocytes, the major component of glial scars, along with other cells in the spinal cord and blood-borne cells leaking from the damaged blood-spinal cord barrier, participate in the process of scar formation. Mature spinal cord injury lesions comprise: (a) a central non-neural lesion core, often referred to as a fibrotic scar, (b) an astroglial scar border that intimately surrounds the lesion core; and (c) a surrounding zone of viable neural tissue that is functional but is demarcated by the presence of reactive glia. Spinal cord injury can be caused by single large lesions or multiple small lesions that span the entire spinal cord.

Current attempts to manage spinal cord injuries involve maintenance of spinal cord nerve cells and regeneration of nerve fibres within the spinal cord, i.e. central nervous regeneration. Medullary implantation of peripheral nerve grafts or re-implantation of avulsed ventral or motor roots into the spinal cord, if performed early after the injury, can curtail some neuronal loss and promote some regrowth of new motor or autonomic axons through spinal cord tissue (central nerve fibre regeneration) and further into the peripheral nerves. However, it has not been possible to reconstruct avulsed dorsal or sensory roots as these nerve fibres are prevented or inhibited from entering into the spinal cord. Today, most patients are not treated at all or receive non-curative palliative procedures which do not give acceptable functional return or pain alleviation. There are currently no effective treatments for spinal cord injuries, which can result in loss of feeling and control of the body below the level of the spinal cord injury.

Stem cells have been extensively studied as neuroregenerative and neuroprotective agents for the treatment of SCI (reviewed by Gazdic et al., Stem Cells Therapy for Spinal Cord Injury, Int. J. Mol. Sci. 2018, 19, 1039). Treatments for mobilisation of endogenous stem cell population are considered to provide a promising therapeutic approach. Results of preclinical studies indicate that application of stem cell-derived progenitors significantly reduces neurological disability in most severe SCIs. However, stem cell transplantation alone is not sufficient to bridge a spinal cord lesion.

As with SCI, there are currently no effective treatments for other neurological injuries, such as brain injury or peripheral nerve injury.

Tendon injuries are some of the most common orthopaedic problems, and cause substantial pain, disability, and time off work. Whilst many tendon injuries are acute, a large number are chronic, degenerative conditions. In either case, repair results in the formation of a fibrovascular scar that never attains the characteristics of normal tendon. Tendon healing is characterised by the formation of fibrovascular scar tissue, as tendon has very little intrinsic regenerative capacity. The molecular mechanisms resulting in scar tissue formation after tendon injuries are not well understood (as reviewed in Schneider et al. Rescue plan for Achilles: Therapeutics steering the fate and functions of stem cells in tendon wound healing; Advanced Drug Delivery Reviews 129 2018 352-375). Briefly, in the first few days after injury a blood clot forms that serves as a preliminary scaffold for invading cells followed by a more robust vascular network which is essential for the survival of tenocytes engaged in the synthesis of new fibrous tissue. Thereafter, fibroblasts are recruited to the injured site and produce initially disorganised extracellular matrix components. Following this a remodelling stage commences characterised by tissue changes resulting in a more fibrous appearance and eventually a scar-like tendon tissue can be observed.

Current biologic treatment strategies have not achieved tendon regeneration but include the use of extracellular matrix patches to provide a scaffold for new cell growth and differentiation (as reviewed in Galatz et al. Tendon Regeneration and Scar Formation: The Concept of Scarless Healing, J. Orthop. Res. 2015, 33(6) 823-831). Platelet rich plasma which comprises a multitude of growth factors normally involved in repair processes has also been investigated for use in tendon repair. However, there is no evidence that either strategy induces tendon regeneration. Tendon injuries are also a particular problem in horses.

There is, therefore, an urgent need to provide more effective treatments for tissue injury, including neurological injury such as spinal cord injury, brain injury, and peripheral nerve injury, and tendon injury, in particular treatments which provide improvements in functional recovery following injury.

The applicant has recognised that scar tissue formation, for example due to accumulation of extracellular matrix proteins such as collagen and/or glycosaminoglycans, prevents improvement in functionality of an injured tissue even after treatment with stem cells or surgery. In fact, administration of stem cells to an injured tissue, for example in SCI, may even increase the formation of scar tissue at the injury site, thereby impairing the repair process and reducing functional recovery. The applicant has appreciated that inhibition of scar tissue formation following tissue injury is a key aspect of the repair process, and in particular is a necessary prerequisite for successful tissue regeneration. The applicant has also recognised that vitamin A may be used to inhibit scar tissue formation, and may thus be used in the effective treatment of tissue injury, including neurological injury (such as spinal cord injury, brain injury, or peripheral nerve injury) and soft tissue injury (such as tendon or ligament injury).

According to the invention, there is provided vitamin A for use in inhibition of scar tissue formation in a subject.

There is also provided according to the invention use of vitamin A in the manufacture of a medicament for the inhibition of scar tissue formation in a subject.

There is further provided according to the invention a method of inhibiting scar tissue formation in a subject comprising administering to the subject an effective amount of vitamin A.

Particular embodiments of the invention relate to inhibition of scar tissue formation occurring other than as a result of infection, for example as a result of injury.

There is also provided according to the invention vitamin A for use in inhibition of scar tissue formation in a subject, following an injury to the subject, for the treatment of the injury.

There is also provided according to the invention use of vitamin A in the manufacture of a medicament for the inhibition of scar tissue formation in a subject, following an injury to the subject, for the treatment of the injury.

There is further provided according to the invention a method of inhibiting scar tissue formation in a subject, following an injury to the subject, for the treatment of the injury comprising administering to the subject an effective amount of vitamin A.

There is also provided according to the invention vitamin A for use in the treatment of an injury to a subject.

There is also provided according to the invention use of vitamin A in the manufacture of a medicament for the treatment of an injury to a subject.

There is further provided according to the invention a method of treating an injury to a subject, comprising administering to the subject an effective amount of vitamin A.

There is further provided according to the invention vitamin A for use in the treatment of tissue damage in a subject.

There is also provided according to the invention use of vitamin A in the manufacture of a medicament for the treatment of tissue damage in a subject.

There is further provided according to the invention a method of treating tissue damage in a subject comprising administering to the subject an effective amount of vitamin A.

Optionally the tissue damage is tissue damage resulting from an injury to the subject.

The injury may be a connective tissue injury, such as a tendon or ligament injury.

Optionally the vitamin A inhibits scar tissue formation by anti-inflammatory action.

Optionally the vitamin A inhibits scar tissue formation by increasing expression of collagenase.

Vitamin A may cause a reduction in the amount of scar tissue already formed after an injury has occurred.

Optionally the scar tissue is actively maintained scar tissue.

Vitamin A is the name of a group of fat-soluble retinoids, including retinol, retinal, and retinyl esters. There are two different categories of vitamin A. The first category, preformed vitamin A, comprises retinol and its esterified form, retinyl ester. The second category, provitamin A, comprises provitamin A carotenoids such as alpha-carotene, beta-carotene and beta-cryptoxanthin. Both retinyl esters and provitamin A carotenoids are converted to retinol, which is oxidized to retinal and then to retinoic acid. Both provitamin A and preformed vitamin A are known be metabolized intracellularly to retinal and retinoic acid, the bioactive forms of vitamin A.

Vitamin A for use according to the invention may be an isolated form of vitamin A. An isolated form of vitamin A is any form of vitamin A found in the diet or a metabolized form thereof. For example, vitamin A may be isolated from fish liver oil. Vitamin A may comprise a preformed vitamin A such as retinol or a retinyl ester. Retinyl esters include retinyl acetate and retinyl palmitate. Vitamin A may comprise a provitamin A, such as a provitamin A carotenoid including alpha-carotene, beta-carotene or beta-cryptoxanthin. Vitamin A may comprise a bioactive form of vitamin A such as retinal or retinoic acid.

Vitamin A is available for human consumption in multivitamins and as a stand-alone supplement, often in the form of retinyl acetate or retinyl palmitate. A portion of the vitamin A in some supplements is in the form of beta-carotene and the remainder is preformed vitamin A; others contain only preformed vitamin A or only beta-carotene. Supplement labels usually indicate the percentage of each form of the vitamin. The amounts of vitamin A in stand-alone supplements range widely. Multivitamin supplements typically contain 2,500 to 10,000 international units (IU) vitamin A, often in the form of both retinol and beta-carotene.

Vitamin A is listed on food and supplement labels in international units (IUs). However, Recommended Dietary Allowance (RDA) (average daily level of intake sufficient to meet the nutrient requirements of nearly all (97%-98%) healthy individuals) for vitamin A is given as micrograms (μg; mcg) of retinol activity equivalents (RAE) to account for the different bioactivities of retinol and provitamin A carotenoids (see Table 1). Because the body converts all dietary sources of vitamin A into retinol, 1 mcg of physiologically available retinol is equivalent to the following amounts from dietary sources: 1 mcg of retinol, 12 mcg of beta-carotene, and 24 mcg of alpha-carotene or beta-cryptoxanthin. From dietary supplements, the body converts 2 mcg of beta-carotene to 1 mcg of retinol.

Conversion rates between mcg RAE and IU are as follows:
1 IU retinol=0.3 mcg RAE;
1 IU beta-carotene from dietary supplements=0.15 mcg RAE;
1 IU beta-carotene from food=0.05 mcg RAE; and
1 IU alpha-carotene or beta-cryptoxanthin=0.025 mcg RAE.

An RAE cannot be directly converted into an IU without knowing the source(s) of vitamin A. For example, the RDA of 900 mcg RAE for adolescent and adult men is equivalent to 3,000 IU if the food or supplement source is preformed vitamin A (retinol). However, this RDA is also equivalent to 6,000 IU of beta-carotene from supplements, 18,000 IU of beta-carotene from food, or 36,000 IU of alpha-carotene or beta-cryptoxanthin from food. So a mixed diet containing 900 mcg RAE provides between 3,000 and 36,000 IU of vitamin A, depending on the foods consumed.

TABLE 1
Recommended Dietary Allowances (RDAs) for Vitamin A

| Age | | Male | Female | Pregnancy | Lactation |
|---|---|---|---|---|---|
| 0-6 | months* | 400 mcg RAE | 400 mcg RAE | | |
| 7-12 | months* | 500 mcg RAE | 500 mcg RAE | | |
| 1-3 | years | 300 mcg RAE | 300 mcg RAE | | |
| 4-8 | years | 400 mcg RAE | 400 mcg RAE | | |
| 9-13 | years | 600 mcg RAE | 600 mcg RAE | | |
| 14-18 | years | 900 mcg RAE | 700 mcg RAE | 750 mcg RAE | 1,200 mcg RAE |
| 19-50 | years | 900 mcg RAE | 700 mcg RAE | 770 mcg RAE | 1,300 mcg RAE |
| 51+ | years | 900 mcg RAE | 700 mcg RAE | | |

*Adequate Intake (AI), equivalent to the mean intake of vitamin A in healthy, breastfed infants.
Source: National Institutes of Health, Vitamin A, Fact Sheet for Health Professionals, as updated 5 Oct. 2018

The Food and Nutrition Board (FNB) at the Institute of Medicine of the National Academies (formerly National Academy of Sciences) has established tolerable Upper Intake Level (UL) (maximum daily intake unlikely to cause adverse health effects) for preformed vitamin A that apply to both food and supplement intakes. The FNB based these ULs on the amounts associated with an increased risk of liver abnormalities in men and women, teratogenic effects, and a range of toxic effects in infants and children. The FNB has not established ULs for beta-carotene and other provitamin A carotenoids.

TABLE 2
Tolerable Upper Intake Levels (ULs) for Preformed Vitamin A*

| Age | | Male | Female | Pregnancy | Lactation |
|---|---|---|---|---|---|
| 0-12 | months | 600 mcg RAE | 600 mcg RAE | | |
| | | (2,000 IU) | (2,000 IU) | | |
| 1-3 | years | 600 mcg RAE | 600 mcg RAE | | |
| | | (2,000 IU) | (2,000 IU) | | |
| 4-8 | years | 900 mcg RAE | 900 mcg RAE | | |
| | | (3,000 IU) | (3,000 IU) | | |
| 9-13 | years | 1,700 mcg RAE | 1,700 mcg RAE | | |
| | | (5,667 IU) | (5,667 IU) | | |
| 14-18 | years | 2,800 mcg RAE | 2,800 mcg RAE | 2,800 mcg RAE | 2,800 mcg RAE |
| | | (9,333 IU) | (9,333 IU) | (9,333 IU) | (9,333 IU) |
| 19+ | years | 3,000 mcg RAE | 3,000 mcg RAE | 3,000 mcg RAE | 3,000 mcg RAE |
| | | (10,000 IU) | (10,000 IU) | (10,000 IU) | (10,000 IU) |

Source: National Institutes of Health, Vitamin A, Fact Sheet for Health Professionals, as updated 5 Oct. 2018
*These ULs, expressed in mcg and in IUs (where 1 mcg = 3.33 IU), only apply to products from animal sources and supplements whose vitamin A comes entirely from retinol or ester forms, such as retinyl palmitate. However, many dietary supplements (such as multivitamins) do not provide all of their vitamin A as retinol or its ester forms. For example, the vitamin A in some supplements consists partly or entirely of beta-carotene or other provitamin A carotenoids. In such cases, the percentage of retinol or retinyl ester in the supplement should be used to determine whether an individual's vitamin A intake exceeds the UL. For example, a supplement labeled as containing 10,000 IU of vitamin A with 60% from beta-carotene (and therefore 40% from retinol or retinyl ester) provides 4,000 IU of preformed vitamin A. That amount is above the UL for children from birth to 13 years but below the UL for adolescents and adults.

The Vitamin A may be provided from a mixture of different sources, including, for example, in normal feed, and in the form of a supplement.

It will be appreciated that use of a natural vitamin for tissue repair is particularly advantageous because of its known safety profile.

Preferably vitamin A for use in inhibition of scar tissue formation in a subject according to the invention comprises a high dose of vitamin A.

A high dose of vitamin A is considered to be a dose that exceeds a UL for the subject. Examples of high doses of vitamin A include: >10,000 IU to 100,000 IU vitamin A per day; about 25,000 to 100,000 IU vitamin A per day; about 50,000 to 100,000 IU vitamin A per day; or about 75,000 to 100,000 IU vitamin A per day, in particular of preformed vitamin A.

Examples of high doses of vitamin A for a horse include: 25,000-250,000, 50,000-250,000, 75,000-250,000, 100,000-250,000, 125,000-250,000, 150,000-250,000, 175,000-250,000, or 200,000-250,000 IU vitamin A per day, or 25,000-50,000, 25,000-75,000, 25,000-100,000, 25,000-125,000, 25,000-150,000, 25,000-175,000, or 25,000-200,000 IU vitamin A per day.

A high dose of vitamin A may be a dose that is 5%-50% of a minimum toxic dose for the subject.

Vitamin A may be administered to the subject once per day, twice per day, three times per day, four times per day, or five times per day.

Vitamin A may be administered to the subject for at least 3 days for example for at least a week, for at least a month, or for at least 6 months from the day of first administration to the subject.

Vitamin A may be administered to the subject for at least five weeks from the day of first administration to the subject.

Vitamin A may be administered to the subject for at least five weeks from the day of first administration to the subject for treatment of an injury that occurred over six months prior to the date of first administration of Vitamin A to the subject in accordance with the invention.

Prolonged exposure to high doses of vitamin A may lead to hypervitaminosis A. Thus, it may be preferred to limit administration of high doses of vitamin A to the subject for up to 6 years, or up to 6 months, from the day of first administration to the subject.

Optionally for an adult human subject (>18 years old), the subject may be administered up to 100,000 IU vitamin A (in particular of preformed vitamin A) per day for up to 6 months. For example, >10,000 IU to 100,000 IU vitamin A per day; about 25,000 to 100,000 IU vitamin A per day; about 50,000 to 100,000 IU vitamin A per day; or about 75,000 to 100,000 IU vitamin A per day (in particular of preformed vitamin A) for up to 6 months.

Optionally for an adult human subject (>18 years old), the subject may be administered up to 25,000 IU vitamin A per day (in particular of preformed vitamin A) for up to 6 years. For example, >10,000 IU to 25,000 IU vitamin A per day (in particular of preformed vitamin A) for up to 6 years

Optionally the subject is administered up to 50% (for example >10% to 50%, or 25% to 50%) of a maximum safe dose of vitamin A (in particular of preformed vitamin A) for the subject per day.

For example, a maximum safe dose of vitamin A (in particular of preformed vitamin A) per day for an adult human subject may be 100,000 IU vitamin A (in particular of preformed vitamin A). Vitamin A toxicity levels have been recorded at around 1,000 IU/kg body weight (BW)/day for a horse. Thus, a maximum safe dose of vitamin A (in particular of preformed vitamin A) per day for an adult horse subject may be 1,000 IU/kg BW vitamin A (in particular of preformed vitamin A).

Optionally for a horse subject, the subject may be administered over 1,000,000 IU vitamin A per day, for example 1,500,000 to 2,000,000 IU vitamin per day.

Optionally the subject may be administered a dose of vitamin A which is upto 50% of a minimum toxic dose for the subject.

Optionally the subject may be administered a dose of vitamin A which is at least 5% of a minimum toxic dose for the subject.

For example, a minimum toxic dose for a subject may be 1,000 IU/kg body weight (BW)/day.

Optionally a horse may be administered a dose of 25,000-250,000, 50,000-250,000, 75,000-250,000, 100,000-250,000, 125,000-250,000, 150,000-250,000, 175,000-250,000, or 200,000-250,000 IU vitamin A per day.

Optionally a horse may be administered a dose of 25,000-50,000, 25,000-75,000, 25,000-100,000, 25,000-125,000, 25,000-150,000, 25,000-175,000, or 25,000-200,000 IU vitamin A per day.

The vitamin A may be administered to a subject systemically for example orally or intravenously.

Optionally vitamin A is used for inhibition of scar tissue formation in the subject resulting from an injury to the subject. The injury may be any injury that causes tissue damage. The injury may be a soft tissue injury which causes damage to soft tissue, such as muscle, a ligament, or a tendon. The injury may be an injury to connective tissue, such as a ligament or a tendon.

Such injuries may be caused, for example, by a strain, sprain, contusion, or a burn. The injury may be a neurological injury which causes damage to neurological tissue, such as a spinal cord injury, a brain injury, or a peripheral nerve injury.

The injury may be a traumatic injury. A traumatic injury is a physical injury of sudden onset and severity which requires immediate medical attention.

Where an injury involves a neurological injury, such as a spinal cord injury or a brain injury, vitamin A may be used to inhibit glial scar tissue formation.

Treatment with vitamin A may begin before surgery to repair the injured tissue.

Vitamin A treatment may begin after surgery to repair the injured tissue.

Vitamin A treatment may begin before surgery and be continued after surgery to repair the injured tissue.

Inhibition of scar tissue formation in accordance with the invention facilities regeneration of normal tissue, in particular by stem cells and/or quiescent cells present within or near a damaged tissue. Such stem cells and quiescent cells may be endogenous to the subject.

Quiescence is the reversible state of a cell in which it does not divide but retains the ability to re-enter cell proliferation. Some adult stem cells are maintained in a quiescent state and can be rapidly activated when stimulated, for example by damage or injury to the tissue in which they reside.

In some embodiments of the invention, one or more regenerative cells may be administered to the subject, for example by injection or transplantation.

Optionally vitamin A is administered to the subject prior to, with, or subsequent to administration of one or more regenerative cells.

There is also provided according to the invention vitamin A for use in inhibition of scar tissue formation in a subject, wherein the vitamin A is to be administered before, with, or after administration of one or more regenerative cells.

There is also provided according to the invention use of vitamin A in the manufacture of a medicament for the inhibition of scar tissue formation in a subject, wherein the vitamin A is to be administered before, with, or after administration of one or more regenerative cells.

There is also provided according to the invention vitamin A for use in the inhibition of scar tissue formation in a subject that has been administered one or more regenerative cells.

There is also provided according to the invention one or more regenerative cells for use in the inhibition of scar tissue formation in a subject that has been administered vitamin A.

There is also provided according to the invention use of vitamin A in the manufacture of a medicament for the inhibition of scar tissue formation in a subject that has been administered one or more regenerative cells.

There is also provided according to the invention use of one or more regenerative cells in the manufacture of a medicament for the inhibition of scar tissue formation in a subject that has been administered vitamin A.

There is also provided according to the invention a method of inhibiting scar tissue formation in a subject, which comprises administering an effective amount of vitamin A and one or more regenerative cells to the subject.

According to the invention there is provided vitamin A and one or more regenerative cells for use in the treatment of an injury to a subject.

There is also provided according to the invention use of vitamin A and one or more regenerative cells in the manufacture of a medicament for the treatment of an injury to a subject.

There is also provided according to the invention vitamin A for use in the treatment of an injury to a subject, wherein the vitamin A is to be administered before, with, or after administration of one or more regenerative cells.

There is also provided according to the invention use of vitamin A in the manufacture of a medicament for the treatment of an injury to a subject, wherein the vitamin A is to be administered before, with, or after administration of one or more regenerative cells.

There is also provided according to the invention vitamin A for use in the treatment of an injury to a subject that has been administered one or more regenerative cells.

There is also provided according to the invention one or more regenerative cells for use in the treatment of an injury to a subject that has been administered vitamin A.

There is also provided according to the invention use of vitamin A in the manufacture of a medicament for the treatment of an injury to a subject that has been administered one or more regenerative cells.

There is also provided according to the invention use of one or more regenerative cells in the manufacture of a medicament for the treatment of an injury to a subject that has been administered vitamin A.

There is also provided according to the invention a method of treating an injury to a subject, which comprises administering an effective amount of vitamin A and one or more regenerative cells to the subject.

According to the invention there is provided vitamin A and one or more regenerative cells for use in the treatment of tissue damage in a subject.

There is also provided according to the invention use of vitamin A and one or more regenerative cells in the manufacture of a medicament for the treatment of tissue damage in a subject.

There is also provided according to the invention vitamin A for use in the treatment of tissue damage in a subject, wherein the vitamin A is to be administered before, with, or after administration of one or more regenerative cells.

There is also provided according to the invention use of vitamin A in the manufacture of a medicament for the treatment of tissue damage in a subject, wherein the vitamin A is to be administered before, with, or after administration of one or more regenerative cells.

There is also provided according to the invention vitamin A for use in the treatment of tissue damage in a subject that has been administered one or more regenerative cells.

There is also provided according to the invention one or more regenerative cells for use in the treatment of tissue damage in a subject that has been administered vitamin A.

There is also provided according to the invention use of vitamin A in the manufacture of a medicament for the treatment of tissue damage in a subject that has been administered one or more regenerative cells.

There is also provided according to the invention use of one or more regenerative cells in the manufacture of a medicament for the treatment of tissue damage in a subject that has been administered vitamin A.

There is also provided according to the invention a method of treating tissue damage in a subject, which comprises administering an effective amount of vitamin A and one or more regenerative cells to the subject.

Optionally the tissue damage is tissue damage resulting from an injury to the subject.

The vitamin A and the one or more regenerative cells may be sourced together or separately.

A regenerative cell is a cell that has capacity to restore at least some functionality to an injured tissue. Examples of regenerative cells include stem cells, progenitor cells, mature cells, and in vitro or ex vivo differentiated stem cells.

Stem cells are cells that can differentiate into other types of cells, and can also divide in self-renewal to produce more of the same type of stem cells. In mammals, there are two broad types of stem cells: embryonic stem cells, which are isolated from the inner cell mass of blastocysts in early embryonic development, and adult stem cells, which are found in various tissues of fully developed mammals.

A progenitor cell is a cell that, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. Most progenitors are described as oligopotent. In this point of view, they may be compared to adult stem cells. But progenitors are said to be in a further stage of cell differentiation. They are in the “centre” between stem cells and fully differentiated cells. Progenitors can go through several rounds of cell division before finally differentiating into a mature cell.

| | Stem Cell | Progenitor Cell |
|---|---|---|
| Self-renewal in vitro | Unlimited | Limited |
| Potentiality | Multipotent | Unipotent, sometimes |
| | | oligopotent |
| Maintenance of self- | Yes | No |
| renewal | | |
| Population | Reaches maximum | Does not reach |
| | number of cells before | maximum population |
| | differentiating | |

In adult organisms, stem cells and progenitor cells replenish adult tissues. In a developing embryo, stem cells can differentiate into all the specialized cells-ectoderm, endoderm and mesoderm but also maintain the normal turnover of regenerative organs, such as blood, skin, or intestinal tissues.

Examples of suitable stem cells include pluripotent stem cells (such as embryonic stem cells or induced pluripotent stem cells), multipotent stem cells, including multipotent stem cells capable of differentiating into neural cells (such as neural stem cells), and multipotent stem cells capable of differentiating into mesenchymal cells (such as mesenchymal stem cells, adipose-derived stem cells or tendon-derived stem cells).

Examples of suitable in vitro or ex vivo differentiated stem cells include in vitro or ex vivo differentiated pluripotent stem cells or in vitro or ex vivo differentiated multipotent stem cells.

Examples of suitable mature cells include neurons, glia and tenocytes.

Vitamin A may be administered before, with, or subsequent to administration of the one or more regenerative cells.

Administration of vitamin A with the one or more regenerative cells may be by co-administration of vitamin A with the one or more regenerative cells, or by simultaneous administration of vitamin A with the one or more regenerative cells (i.e. by separate administration at the same time, for example by different routes of administration).

Vitamin A may be administered to the subject before administration of the one or more regenerative cells. For example, the one or more regenerative cells may be administered within 6, 12, 24, 48, 72, or 96 hours after administration of vitamin A.

In other embodiments, vitamin A may be administered to the subject after administration of the one or more regenerative cells. For example, vitamin A may be administered within 6, 12, 24, 48, 72, or 96 hours of administration of the one or more regenerative cells.

It will be appreciated that vitamin A should preferably be administered as soon as possible after an injury has occurred. Optionally vitamin A is administered within a month, within a week, or within a day of the injury. Optionally vitamin A is administered with a week of the injury.

However, beneficial effects of treatment with vitamin A have also been observed when treatment is initiated many months or even years after an injury has occurred. Thus, optionally vitamin A may be administered months, years or even decades after an injury has occurred, for example within six months, a year, or a decade, or within twenty, thirty, forty, fifty, or sixty years of the injury.

The one or more regenerative cells may comprise one or more autologous cells. For example, a stem cell, progenitor, or mature cell may be taken from a subject, and optionally expanded and/or differentiated in vitro or ex vivo, before being administered back into the subject to aid restoration of at least some functionality to an injured tissue.

There are three known accessible sources of autologous adult stem cells in humans: bone marrow, adipose tissue, and blood. Adult stem cells may also be isolated from dental pulp, skin and most major organs and tissues of the body.

The one or more regenerative cells may comprise one or more allogeneic cells. For example, a stem cell or a mature cell may be taken from a healthy subject, and optionally expanded and/or differentiated in vitro or ex vivo, before being administered to the subject in need thereof to aid restoration of at least some functionality to an injured tissue.

In some cases, the one or more regenerative cells comprises one or more stem cells. Stem cells can be totipotent (i.e. they can differentiate into all the cell types in a body as well as the extraembryonic, or placental, cells); pluripotent (i.e. they can differentiate into all the cell types in a body); multipotent (i.e. they can differentiate into more than one cell type, but are more limited that pluripotent stem cells); or unipotent (i.e. they can differentiate into only one cell type).

Examples of pluripotent stem cells are embryonic stem cells (ESCs) which can be derived from the inner cell mass of embryos, including by parthogenesis, and induced pluripotent stem cells (iPSCs). Mature adult cells may be directed to a pluripotent state by addition of specific factors to the cells in vitro. These resulting cells are termed iPSCs. An iPSC can differentiate into all cell types in a body.

Therefore, in some cases the one or more stem cells of the invention may comprise one or more pluripotent stem cells such as one or more ESC or iPSC.

It may be advantageous to use multipotent stem cells for regenerative purposes. Mesenchymal stem cells (MSCs) are a type of multipotent stem cell found in large numbers in adults that are relatively easily isolated from bone marrow, adipose tissue and perivascular niches. MSCs and other multipotent stem cells such as tendon-derived stem cells may be particularly advantageous for restoring function to injured tendons. However, the differentiation potential of multipotent stem cells is more restricted than that of pluripotent stem cells.

In some cases the one or more stem cells may comprise one or more multipotent stem cells capable of differentiating into one or more neural cells, for example a neural stem cell. The neural cells may include neurons and/or glia.

In some cases the one or more stem cells may comprise one or more multipotent stem cells capable of differentiating into one or more mesenchymal cells, for example a mesenchymal stem cell, adipose-derived stem cell or tendon-derived stem cell. The mesenchymal cell may include tenocytes.

There are several different methods to differentiate stem cells to specific cell lineages. For example, a stem cell may be cultured in vitro or ex vivo in the presence of specific growth factors and/or morphogenic factors and/or small molecules (collectively termed ‘differentiation factors’) for specific times to direct differentiation to a specific cell lineage. The specific differentiation factor or combination thereof and the specific time of exposure to said factors will depend on the identity of the starting stem cell and the specific cell lineage required.

Mechanical factors may also be used control the ability of stem cells to differentiate towards specific lineages. For example, a substrate with a specific stiffness may be selected to control stem cell differentiation. Substrates with varying surface chemistry (for example hydrophobic/hydrophilic properties) and topography (for example the degree of folding of a particular substrate) may be selected to control stem cell differentiation.

Stem cells may also be differentiated to specific cell lineages by culturing the stem cells on or within naturally-derived biomaterials for example 3D collagen gels or decellularised scaffolds isolated from an in vivo microenvironment.

Differentiation protocols towards specific cell lineages for specific starting stem cells are well known to the person skilled in the art and may use a combination of the approaches outlined above. For example, differentiation of stem cells for tendon and spinal cord regeneration is reviewed in Lui Stem cell technology for tendon regeneration: current status, challenges, and future research directions Stem Cells and Cloning: Advances and Applications 2015:8 163-174 and briefly reviewed in Gazdic et al., Stem Cells Therapy for Spinal Cord Injury, Int. J. Mol. Sci. 2018, 19, 1039.

The one or more regenerative cells may comprise one or more in vitro- or ex vivo-differentiated stem cells for example neurons, glia, and/or tenocytes. In some cases, stem cells may be cultured and maintained as stem cells in vitro or ex vivo for example, to increase the number of stem cells compared to the starting number of stem cells, before being administered as stem cells into the subject in need thereof. Stem cells may be isolated from a heathy subject or tissue and stored before being administered to a subject in need thereof. Stem cells may be isolated from a healthy subject or tissue and optionally sorted, for example using flow-assisted cell sorting, before being immediately administered to a subject in need thereof.

The one or more regenerative cells may comprise one or more mature cells for example neurons, glia, and/or tenocytes.

Regenerative cells may be isolated from a heathy subject or tissue and stored before being administered to a subject in need thereof. Regenerative cells may be isolated from a healthy subject or tissue and optionally sorted, for example using flow-assisted cell sorting, before being immediately administered to a subject in need thereof. Regenerative cells may be cultured in vitro or ex vivo for example to increase the number of regenerative cells compared with the starting number of regenerative cells and/or to differentiate the regenerative cells to specific cell lineages (such as neurons, glia or tenocytes).

The one or more regenerative cells may be administered locally i.e. local to a site of injury that is in need of repair. For example, a regenerative cell may be injected or transplanted into a tendon in need of repair and/or into one or more lesions of a tendon in need of repair; a regenerative cell may be administered by a direct intramedullary or intrathecal injection or transplantation of a spinal cord in need of repair and/or into one or more lesions of a spinal cord in need of repair. Alternatively, a regenerative cell may be administered distally i.e. distal to a site of injury that is in need of repair. For example a regenerative cell may be injected intravenously and/or intraperitoneally and/or subcutaneously for tendon and/or spinal cord repair. Alternatively, regenerative cells may be administered both locally and distally, or locally and systemically, or distally and systemically, or locally and distally and systemically. Routes of administration for regenerative cells have been reviewed in Lui Stem cell technology for tendon regeneration: current status, challenges, and future research directions Stem Cells and Cloning: Advances and Applications 2015:8 163-174 and Oh et al. Current Concept of Stem Cell Therapy for Spinal Cord Injury: A Review 2016; 12(2):40-46.

Regenerative cells may be administered to a subject in need thereof at the same time as surgery takes place to repair the injured tissue. Regenerative cells may be administered to a subject in need thereof after surgery to repair the injured tissue.

Regenerative cells may be administered to a subject in need thereof multiple times. Each regenerative cell administration subsequent to the initial regenerative cell administration may be termed a “top up”. Each top up may be administered via the same or a different route as the initial administration. Thus a top up may be administered locally. Alternatively, a top up may be administered distally.

The top up administration may be up to one year later than the initial regenerative cell administration. For example, the top up administration may be up to six months later than the initial regenerative cell administration; up to three months later than the initial regenerative cell administration; up to one month later than the initial regenerative cell administration; or up to one week later than the initial regenerative cell administration.

As regenerative cells may be administered to a subject in need thereof multiple times, each top up administration may take place at regular time intervals. For example, each top up administration may take place weekly, monthly, quarterly, bi-annually, or annually.

Each top up administration may be with any regenerative cell. For example, each top up administration may be with the same regenerative cell as the initial regenerative cell administered. Alternatively, each top up administration may be with a different regenerative cell to the initial regenerative cell administered.

Also provided according to the invention is a multiple-dose formulation comprising a plurality of unit doses of vitamin A wherein each unit dose comprises up to 100,000 IU vitamin A, for example >10,000 IU to 100,000 IU vitamin A; 25,000 to 100,000 IU vitamin A; 50,000 to 100,000 IU vitamin A; or 75,000 to 100,000 IU vitamin A (in particular of preformed vitamin A).

There is also provided according to the invention a multiple-dose formulation which comprises a plurality of separate unit doses of vitamin A, wherein each unit dose comprises 25,000-250,000, 50,000-250,000, 75,000-250,000, 100,000-250,000, 125,000-250,000, 150,000-250,000, 175,000-250,000, or 200,000-250,000 IU vitamin A (in particular of preformed vitamin A).

There is also provided according to the invention a multiple-dose formulation which comprises a plurality of separate unit doses of vitamin A, wherein each unit dose comprises 25,000-50,000, 25,000-75,000, 25,000-100,000, 25,000-125,000, 25,000-150,000, 25,000-175,000, or 25,000-200,000 IU vitamin A (in particular of preformed vitamin A).

There is also provided according to the invention a multiple-dose formulation of the invention for administration to a horse.

Vitamin A of a multiple-dose formulation of the invention may comprise any combination of vitamin A described previously.

A multiple-dose formulation of the invention may comprise at least 7 unit doses, at least 30 unit doses, or at least 100 unit doses of vitamin A.

Each unit dose of vitamin A in a multiple-dose formulation of the invention may comprise a pharmaceutical composition comprising vitamin A and a pharmaceutically acceptable carrier, excipient or diluent.

Optionally the pharmaceutical composition is a sterile composition.

There is also provided according to the invention a multiple-dose formulation for use in inhibition of scar tissue formation in a subject.

Vitamin A and the one or more regenerative cells may be provided as a combined preparation.

According to the invention there is provided a combined preparation comprising: (a) vitamin A; and (b) one or more regenerative cells.

The term “combined preparation” as used herein refers to a “kit of parts” in the sense that the combination components (a) and (b) as defined above can be dosed independently or by use of different fixed combinations with distinguished amounts of the combination components (a) and (b). The components can be administered simultaneously or one after the other. If the components are administered one after the other, preferably the time interval between administration is chosen such that the therapeutic effect of the combined use of the components is greater than the effect which would be obtained by use of only any one of the combination components (a) and (b).

The components of the combined preparation may be present in one combined unit dosage form, or as a first unit dosage form of component (a) and a separate, second unit dosage form of component (b). The ratio of the total amounts of the combination component (a) to the combination component (b) to be administered in the combined preparation can be varied, for example in order to cope with the needs of a single patient, which can be due, for example, to the particular condition, age, sex, or body weight of the patient.

A combined preparation of the invention may be provided as a pharmaceutical combined preparation for administration to a mammal, preferably a human, or a non-human mammal such as a horse, or a dog. The vitamin A may optionally be provided together with a pharmaceutically acceptable carrier, excipient, or diluent, and/or the one or more regenerative cells may optionally be provided together with a pharmaceutically acceptable carrier, excipient, or diluent.

The combined preparation may comprise up to 100,000 IU vitamin A. The combined preparation may comprise any combination of vitamin A described previously.

Use of vitamin A, optionally with one or more regenerative cells, in accordance with the invention may be particularly effective for the treatment of older subjects. For example, a human subject may be at least 18 years old, at least 25 years old, at least 30 years old, at least 40 years old, or at least 50 years old.

The subject may be a non-human subject, in particular a mammal such as a horse, or a dog.

Uses and methods of the invention may be particularly advantageous for the treatment of connective tissue injury, such as tendon or ligament injury in a horse, or for the treatment of a neurological injury (for example, a spinal cord injury) in a dog.

### Maintenance Doses

We have found that administering a post-treatment maintenance dose of vitamin A supplement provides continued benefits, especially for treatment of soft tissue injuries, for example connective tissue injuries. As described in more detail in Example 10 below, after several weeks of treatment with “full-treatment doses” of vitamin A supplement, horses with connective tissue injuries (tendon or ligament) were orally administered vitamin A once per day with a reduced, “maintenance dose” of half the “full-treatment dose” of vitamin A supplement for 7 weeks. The results provide evidence for beneficial effects of continuing with a maintenance dose of vitamin A supplement after a period of administration of full-treatment doses.

Accordingly, after a period of treatment of a subject with “full-treatment doses” of Vitamin A, it may be advantageous to continue treatment for a further period with “maintenance doses” of Vitamin A.

A “maintenance dose” is a dose of vitamin A, or of a composition comprising vitamin A, which is less than a “full-treatment dose”.

Optionally a maintenance dose is up to three quarters of a full-treatment dose, or up to two-thirds of a full treatment dose.

Optionally a maintenance dose is at least a quarter of a full-treatment dose.

Optionally a maintenance dose is to be administered after the subject has been administered one or more full-treatment doses.

Optionally a maintenance dose is to be administered from the day after the last administration of a full-treatment dose.

Typically, a plurality of maintenance doses is to be administered to the subject.

Optionally the maintenance doses are to be administered for at least four weeks from the day of first administration of a maintenance dose to the subject.

Optionally the maintenance doses are to be administered for at least 12 weeks from the day of first administration of a maintenance dose to the subject.

Optionally the maintenance doses are to be administered for at least 6 months from the day of first administration of a maintenance dose to the subject.

Optionally the maintenance doses are to be administered for at least 12 months from the day of first administration of a maintenance dose to the subject.

Optionally the maintenance doses are to be administered for up to 6 years from the day of first administration of a maintenance dose to the subject.

Optionally the subject is a human subject.

For a human subject the, or each full-treatment dose optionally comprises >10,000 to 100,000 IU vitamin A per day.

For a human subject the, or each full-treatment dose optionally comprises about 25,000-100,000, 50,000-100,000, or 75,000-100,000 IU vitamin A per day

For a human subject the, or each maintenance dose optionally comprises >2,500 IU to 75,000 IU vitamin A per day.

For a human subject optionally the, or each maintenance dose comprises 5,000-75,000, 10,000-75,000, or 20,000-75,000 IU vitamin A per day.

For a human subject optionally the, or each maintenance dose comprises 5,000-50,000, 10,000-50,000, or 20,000-50,000 IU vitamin A per day.

Optionally the subject is a non-human subject.

Optionally the subject is a horse.

For a horse, optionally the, or each full-treatment dose comprises 25,000-250,000, 50,000-250,000, 75,000-250,000, 100,000-250,000, 125,000-250,000, 150,000-250,000, 175,000-250,000, or 200,000-250,000 IU vitamin A per day.

For a horse, optionally the, or each maintenance dose comprises 10,000-200,000, 20,000-150,000, 40,000-100,000, or 60,000-100,000 IU vitamin A per day.

A “maintenance dose” may be administered as a single dose, or in multiple dose units. For example, a maintenance dose of 80,000 IU vitamin A per day for a horse may be provided as two doses of 40,000 IU vitamin A, one dose to be given in the morning, and another dose to be given in the evening.

Similarly, a “full-treatment dose” may be administered as a single dose, or in multiple dose units. For example, a full-treatment dose of 160,000 IU vitamin A per day for a horse may be provided as two doses of 80,000 IU vitamin A, one dose to be given in the morning, and another dose to be given in the evening.

There is also provided according to the invention a multiple-dose formulation which comprises a plurality of separate unit doses of vitamin A, wherein each unit dose is a maintenance dose of vitamin A, and wherein each unit dose comprises >2,500 IU to 75,000 IU vitamin A.

Optionally the, or each unit dose comprises 5,000-75,000, 10,000-75,000, or 20,000-75,000 IU vitamin A.

Optionally the, or each unit dose comprises 5,000-50,000, 10,000-50,000, or 20,000-50,000 IU vitamin A.

Optionally the, or each unit dose of a multiple-dose formulation of the invention is for administration to a human subject.

There is also provided according to the invention a multiple-dose formulation which comprises a plurality of separate unit doses of vitamin A, wherein each unit dose is a maintenance dose of vitamin A, and wherein each unit dose comprises 10,000-200,000, 20,000-150,000, 40,000-100,000, or 60,000-100,000 IU vitamin A.

Optionally the, or each unit dose of a multiple-dose formulation of the invention is for administration to a horse.

Optionally the vitamin A comprises isolated vitamin A.

Optionally the vitamin A comprises a preformed vitamin A, such as a retinyl ester or retinol.

Optionally the vitamin A comprises a provitamin A, such as a carotenoid.

Optionally the vitamin A comprises a bioactive form of vitamin A, such as retinal or retinoic acid.

Optionally the vitamin A is part of a pharmaceutical composition comprising vitamin A and a pharmaceutically acceptable carrier, excipient or diluent.

Optionally the composition is a sterile composition.

Optionally the vitamin A is the only non-cellular, non-antibiotic, active agent present in the pharmaceutical composition.

Optionally a multiple-dose formulation of the invention comprises at least 7, at least 30, or at least 100 separate unit doses of vitamin A.

There is also provided according to the invention a multiple-dose formulation which comprises: a first plurality of separate unit doses of vitamin A, wherein each unit dose of the first plurality of separate unit doses is a full-treatment unit dose of vitamin A; and a second plurality of separate unit doses of vitamin A, wherein each unit dose of the second plurality of separate unit doses is a maintenance dose of vitamin A.

A multiple-dose formulation of the invention comprising a first plurality of separate unit doses and a second plurality of unit doses may be for treatment of a human subject.

For a human subject optionally each maintenance unit dose optionally comprises >2,500 IU to 75,000 IU vitamin A.

For a human subject optionally each maintenance unit dose comprises 5,000-75,000, 10,000-75,000, or 20,000-75,000 IU vitamin A.

For a human subject optionally each maintenance unit dose comprises 5,000-50,000, 10,000-50,000, or 20,000-50,000 IU vitamin A.

A multiple-dose formulation of the invention comprising a first plurality of separate unit doses and a second plurality of unit doses may be for treatment of a non-human subject.

Optionally the subject is a horse.

For a horse, optionally each maintenance unit dose comprises 10,000-200,000, 20,000-150,000, 40,000-100,000, or 60,000-100,000 IU vitamin A.

The term “unit dose” as used herein refers to physically discrete units suited as unitary doses for the subject to be treated. That is, the vitamin A (or composition comprising vitamin A) is formulated into discrete dose units each containing a predetermined “unit dose” quantity of vitamin A calculated to produce the desired therapeutic effect, typically in association with a required pharmaceutical carrier, excipient or diluent. It should be noted that, in some cases, two or more individual dose units in combination provide a therapeutically effective amount of the active ingredient, for example, two tablets or capsules taken together (or sequentially) may provide a therapeutically effective dose, such that the unit dose in each tablet or capsule is approximately 50% of the therapeutically effective amount.

Each unit dose of a multiple-dose formulation of the invention is typically provided as a sterile unit dose.

A multiple-dose formulation of the invention may be provided packaged in a container. The container can be, for example, a bottle (e.g., with a closure device, such as a cap), a blister pack (e.g., which can provide for enclosure of one or more doses per blister), a vial, flexible packaging (e.g., sealed Mylar or plastic bags), an ampule (for single doses in solution), a dropper, a syringe, thin film, a tube and the like. In some embodiments, a container, such as a sterile container, comprises a subject pharmaceutical composition. In some embodiments the container is a bottle or a syringe. In some embodiments the container is a bottle. In some embodiments the container is a syringe.

For example, each unit dose of the multiple-dose formulation may be provided in a separate well or blister of the container, with a foil seal covering each well/blister. In addition to the container containing the unit doses of the multiple-dose formulation, an information package insert may be included describing the use and attendant benefits of the active ingredient (for example, vitamin A or a composition comprising vitamin A) in treating the condition of interest (for example, tissue damage). Preferred compounds, compositions, and unit doses are those described herein.

There is also provided according to the invention a multiple-dose formulation of the invention for use as a medicament.

There is also provided according to the invention a multiple-dose formulation of the invention for use in inhibition of scar tissue formation in a subject.

There is also provided according to the invention use of a multiple-dose formulation of the invention in the manufacture of a medicament for inhibition of scar tissue formation in a subject.

There is also provided according to the invention a multiple-dose formulation of the invention for use in the treatment of tissue damage in a subject.

There is also provided according to the invention use of a multiple-dose formulation of the invention in the manufacture of a medicament for the treatment of tissue damage in a subject.

There is also provided according to the invention a multiple-dose formulation of the invention for use in the treatment of an injury to the subject.

There is also provided according to the invention use of a multiple-dose formulation of the invention in the manufacture of a medicament for the treatment of an injury to the subject.

Optionally the injury comprises a soft tissue injury, such as a tendon injury or a ligament injury.

The vitamin A and/or the one or more regenerative cells can be incorporated into a variety of formulations for therapeutic administration, more particularly by combination with appropriate, pharmaceutically acceptable carriers, pharmaceutically acceptable diluents, or other pharmaceutically acceptable excipients, and can be formulated into preparations in solid, semi-solid, liquid or gaseous forms, such as tablets, capsules, powders, granules, ointments, solutions, suppositories, injections, inhalants and aerosols as appropriate.

Optionally the vitamin A is in solid form.

Optionally the vitamin A is not in an organic solution.

Optionally the vitamin A is not encapsulated by, or attached to a microparticle.

Optionally the vitamin A is not encapsulated by, or attached to a nanoparticle.

Vitamin A can be administered in the form of a pharmaceutically acceptable salt. It can also be used alone or in appropriate association, as well as in combination, with other pharmaceutically active compounds. Optionally vitamin A is administered with an antibiotic agent. Optionally vitamin A is the only non-cellular, non-antibiotic, active agent administered. The following methods and excipients are merely exemplary and are in no way limiting.

For oral preparations, vitamin A can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Vitamin A and/or the one or more regenerative cells can be formulated into preparations for injection by dissolving, suspending or emulsifying in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, a pharmaceutical composition of the present disclosure can comprise further agents such as dopamine or psychopharmacologic drugs, depending on the intended use of the pharmaceutical composition.

Pharmaceutical compositions are prepared by mixing Vitamin A having the desired degree of purity, and/or the one or more regenerative cells with optional physiologically acceptable carriers, other excipients, stabilizers, surfactants, buffers and/or tonicity agents. Acceptable carriers, other excipients and/or stabilizers are nontoxic to recipients at the dosages and concentrations employed, and may include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

An aqueous formulation can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the formulation to reduce aggregation and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant can range from about 0.001% to about 1% w/v.

A lyoprotectant can also be added in order to protect a labile active ingredient against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Unit dosage forms for oral administration such as syrups, elixirs, and suspensions can be provided wherein each dosage unit, for example, teaspoonful, tablespoonful, or tablet contains a predetermined amount of the active agent (i.e. vitamin A and/or the one or more regenerative cells). Similarly, unit dosage forms for injection or intravenous administration can comprise vitamin A and/or the one or more regenerative cells in a composition as a solution in sterile water, normal saline or another pharmaceutically acceptable carrier.

The term “unit dosage form,” (or “unit dose”), as used herein, refers to physically discrete units suitable as unitary dosages for human and animal subjects, each unit containing a predetermined quantity of vitamin A and/or the one or more regenerative cells, calculated in an amount sufficient to produce the desired effect in association with a pharmaceutically acceptable diluent, carrier or vehicle.

Vitamin A and/or the one or more regenerative cells can be administered as an injectable formulation. Typically, injectable compositions are prepared as liquid solutions or suspensions; solid forms suitable for solution in, or suspension in, liquid vehicles prior to injection can also be prepared.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985. The composition or formulation to be administered will, in any event, contain a quantity of vitamin A and/or the one or more regenerative cells adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Ranges may be expressed herein as from “about” one particular value, and/or to another particular value. When such a range is expressed, another embodiment includes from the one particular value and/or to the other particular value. Similarly, when values are expressed as approximations, by the use of the antecedent “about”, it will be understood that the particular value forms another embodiment.

Wherever the term “vitamin A” is used herein this includes reference to “vitamin A or a pharmaceutically acceptable salt thereof”.

Optionally uses and methods of the invention are limited to cosmetic treatment of a subject.

Optionally uses and methods of the invention are for non-cosmetic treatment of a subject.

Methods for visualising scar tissue formation are known to those of ordinary skill in the art. Examples include MRI for spinal cord, and tendons, and ultrasound in tendons. Suitable examples are described in the following documents:

### MRI in Spinal Cord Injury:

Ellingson et al., “Imaging Techniques in Spinal Cord Injury”, World Neurosurg. 2014 December; 82(6): 1351-1358;
Hu et al., “Glial scar and neuroregeneration: histological, functional, and magnetic resonance imaging analysis in chronic spinal cord injury”, Journal of Neurosurgery, 2010, 13(2) (doi.org/10.3171/2010.3.SPINE09190);
Byrnes et al., “Neuropathological Differences Between Rats and Mice after Spinal Cord Injury”, J Magn Reson Imaging. 2010 October; 32(4): 836-846.

### MRI in Tendon Injury:

Galatz et al., “Tendon Regeneration and Scar Formation: The Concept of Scarless Healing”, Journal Of Orthopaedic Research, 2015, 823-831;
Tavares Jr, et al., “Healing of the Achilles tendon in rabbits—evaluation by magnetic resonance imaging and histopathology”, Journal of Orthopaedic Surgery and Research, volume 9, Article number: 132 (2014);

### Ultrasound in Tendon Injury:

Ackerman, et al., “Non-Invasive Ultrasound Quantification of Scar Tissue Volume Identifies Early Functional Changes During Tendon Healing”, Volume 37, Issue 11, November 2019, Pages 2476-2485;
Galatz et al., “Tendon Regeneration and Scar Formation: The Concept of Scarless Healing”, Journal Of Orthopaedic Research, 2015, 823-831

### Treatment of Pulmonary Fibrosis

A second aspect of the invention relates to the treatment of pulmonary fibrosis.

This second aspect of the invention relates to compounds and compositions for use in the treatment of pulmonary fibrosis, including pulmonary fibrosis caused by respiratory infection, and to methods of treatment of pulmonary fibrosis using the compounds and compositions.

Damage to tissues can result from various stimuli, including infections, autoimmune reactions, allergic responses, toxins, radiation and mechanical injury. The repair process typically involves two distinct phases: a regenerative phase, in which injured cells are replaced by cells of the same type, leaving no lasting evidence of damage; and a phase known as fibrosis, in which connective tissue replaces normal parenchymal tissue. Although initially beneficial, the repair process becomes pathogenic when it is not controlled appropriately, resulting in substantial deposition of extracellular matrix (ECM) components in which normal tissue is replaced with permanent scar tissue. In some diseases, extensive tissue remodelling and fibrosis can ultimately lead to organ failure and death.

Fibrosis is initiated when immune cells, such as macrophages, release soluble factors (such as TGF-β) that stimulate fibroblasts. These pro-fibrotic factors initiate signal transduction pathways, such as the AKT/mTOR and SMAD pathways, that ultimately lead to the proliferation and activation of fibroblasts, which deposit extracellular matrix into the surrounding connective tissue. ECM synthesis and degradation is tightly regulated, ensuring maintenance of normal tissue architecture. However, this process can lead to a progressive irreversible fibrotic response if tissue injury is severe or repetitive, or if the wound healing response itself becomes deregulated.

The key cellular mediator of fibrosis is the myofibroblast, which when activated serves as the primary collagen-producing cell. Myofibroblasts are generated from a variety of sources including resident mesenchymal cells, epithelial and endothelial cells in processes termed epithelial/endothelial-mesenchymal transition, as well as from circulating fibroblast-like cells called fibrocytes that are derived from bone-marrow stem cells. Myofibroblasts are activated by a variety of mechanisms, including paracrine signals derived from lymphocytes and macrophages, autocrine factors secreted by myofibroblasts, and pathogen-associated molecular patterns (PAMPS) produced by pathogenic organisms that interact with pattern recognition receptors on fibroblasts. Cytokines (IL-13, IL-21, TGF-β1), chemokines (MCP-1, MIP-1B), angiogenic factors (VEGF), growth factors (PDGF), peroxisome proliferator-activated receptors (PPARs), acute phase proteins (SAP), caspases, and components of the renin-angiotensin-aldosterone system (ANG II) have been identified as important regulators of fibrosis.

In pulmonary fibrosis, gradual exchange of normal lung parenchyma with fibrotic tissue causes an irreversible decrease in oxygen diffusion capacity as the lungs become scarred over time. Symptoms of pulmonary fibrosis include shortness of breath (particularly with exertion), chronic dry, hacking coughing, fatigue and weakness, chest discomfort including chest pain, loss of appetite and rapid weight loss. Complications may include pulmonary hypertension, respiratory failure, pneumothorax, and lung cancer.

Pulmonary fibrosis may be a secondary effect of other diseases or conditions (many of which are classified as interstitial lung diseases), including: infections, autoimmune diseases, connective tissue diseases, other diseases involving connective tissue, certain medications, radiation therapy, or inhalation of environmental and occupational pollutants. Cigarette smoking can increase the risk or make the illness worse. There have been reports of development of pulmonary fibrosis in patients infected with Severe acute respiratory syndrome coronavirus 2 (SARS-COV-2), the causative agent of coronavirus disease 2019 (COVID-19) (see, for example, Shi et al., Lancet Infect Dis 2020; 20: 425-34; see also Wadman et al., “How does coronavirus kill? Clinicians trace a ferocious rampage through the body, from brain to toes”, Science, Apr. 17, 2020, doi:10.1126/science.abc3208).

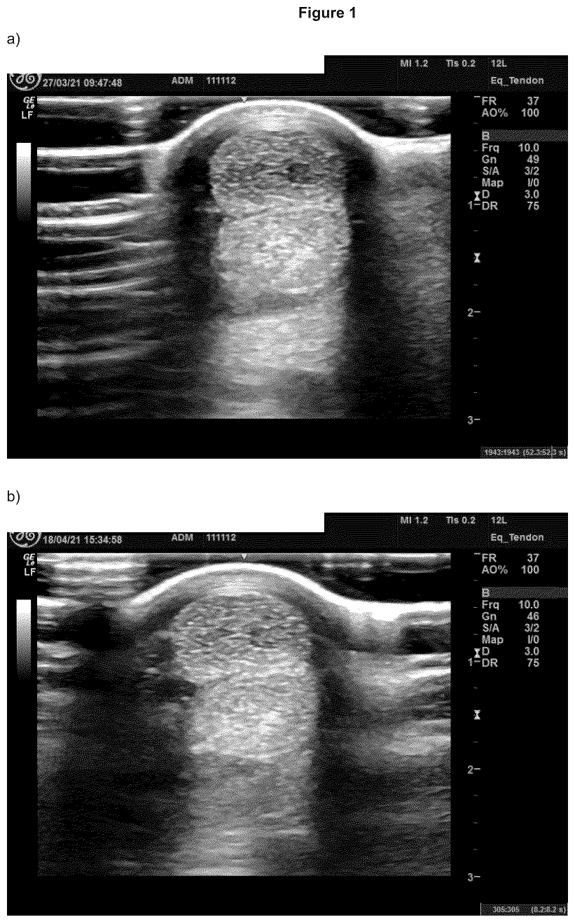

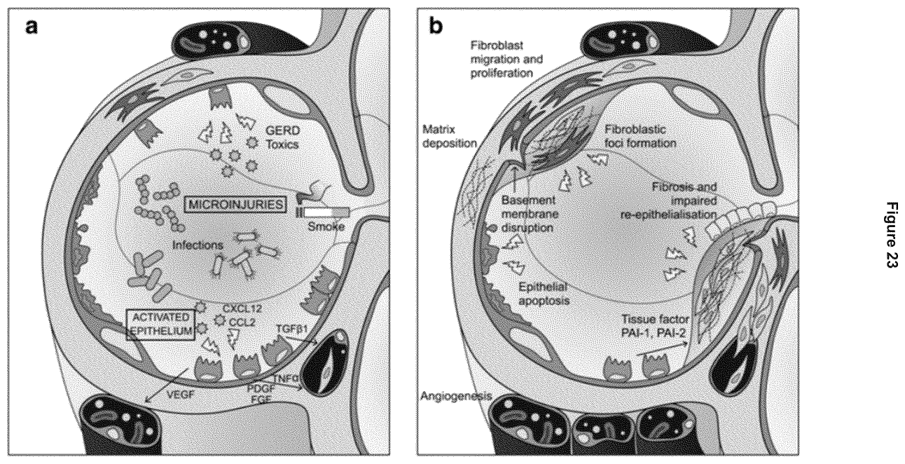

Pulmonary fibrosis can also appear, however, without any known cause. Idiopathic pulmonary fibrosis (IPF) is the most common type of pulmonary fibrosis. About 5 million people are affected globally, with those in their 60s and 70s most commonly affected. Recent evidence supports the hypothesis that virus infection could play a key role in pathogenesis of IPF (Sheng et al “Viral Infection Increases the Risk of Idiopathic Pulmonary Fibrosis”, Chest Journal (2019); online article: doi.org/10.1016/j.chest.2019.10.032). The authors conclude that the presence of persistent or chronic viral infections, including Epstein-Barr virus (EBV), cytomegalovirus (CMV), human herpesvirus 7 (HHV-7), and human herpesvirus 8 (HHV-8), significantly increases the risk of developing IPF (but not exacerbation of IPF), implying that viral infection could be a potential risk factor for IPF. There is also evidence which points to a genetic predisposition to IPF in a subset of patients. For example, a mutation in surfactant protein C (SP-C) has been found to exist in some families with a history of pulmonary fibrosis. Autosomal dominant mutations in the TERC or TERT genes, which encode telomerase, have been identified in about 15 percent of pulmonary fibrosis patients. The factors driving the course of pulmonary fibrosis are not definitively identified. However, repeated microinjury to alveolar epithelial tissues is considered to be the first trigger of an aberrant repair process in which several lung cells develop abnormal behaviours that promote the fibrotic process. A dysfunctional, ageing lung epithelium exposed to recurrent microinjuries leads to defective attempts of regeneration and aberrant epithelial-mesenchymal crosstalk, creating an imbalance between profibrotic and antifibrotic mediators. Environments supportive of exaggerated fibroblast and myofibroblast activity are maintained, and the normal repair mechanisms are replaced with chronic fibrosis. Myofibroblasts, considered the effector cells of fibrogenesis, synthesise an abnormally stiff extracellular matrix. Mechanisms of pulmonary fibrosis are reviewed by Kinoshita and Goto (Int. J. Mol. Sci. 2019, 20, 1461; see FIG. 1), and Sgalla et al (Respiratory Research (2018) 19:32). A schematic view of IPF pathogensis is provided in FIG. 23 (adapted from FIG. 1 of Sgalla et al).

There is no known cure for pulmonary fibrosis. IPF is a progressive disease with a 5-year survival rate of only 20%, reflecting the lack of effective therapies. Treatment is aimed at improving symptoms, and may include oxygen therapy and pulmonary rehabilitation. Certain medications may be used to try to slow the worsening of scarring. Immune suppressive agents, such as corticosteroids, may be used to decrease lung inflammation and subsequent scarring. However, responses to treatment are variable. Anti-inflammatory agents also have only limited success in reducing the fibrotic process. Therapeutic reagents pirfenidone and nintedanib were developed to slow the progression of pulmonary fibrosis. Whilst these agents are effective and well-tolerated (Hughes et al., “Real World Experiences: Pirfenidone and Nintedanib are Effective and Well Tolerated Treatments for Idiopathic Pulmonary Fibrosis”, J Clin Med. 2016 September; 5(9): 78), they do not improve lung function and patients often remain with poor pulmonary function. Lung transplantation may be the only therapeutic option available in severe cases.

An adequate vitamin A intake is required in early lung development, alveolar formation, tissue maintenance and regeneration. Timoneda et al. (“Vitamin A Deficiency and the Lung”, Nutrients 2018; 10, 1132) review vitamin A deficiency (VAD) and the lung. Chronic VAD has been associated with histopathological changes in the pulmonary epithelial lining that disrupt the normal lung physiology, predisposing to severe tissue dysfunction and respiratory diseases. In addition, there are important alterations of the structure and composition of extracellular matrix (ECM) with thickening of the alveolar basement membrane (BM) and ectopic deposition of collagen I.

Pulmonary fibrosis is characterized by a replacement of normal lung parenchyma with fibrotic tissue accompanied by inflammation and excessive collagen deposition. The most prominent characteristic in the pathogenesis of lung fibrosis is persistent alveolitis, accumulation of myofibroblasts and the deposition of excessive amounts of ECM. Myofibroblasts (fibroblasts that express some features of muscle differentiation) are derived from resident mesenchymal cells, bone marrow progenitors (fibrocytes) and epithelial cells that have undergone epithelial-mesenchymal transition (EMT). One of the primary functions of the ECM is to maintain tissue integrity and homeostasis of multicellular organisms and it plays an important role in regulating alveolarization, tissue repair and remodelling in pulmonary tissue. Therefore, changes in the structure or composition of the ECM can induce alterations in cell and organ responses, leading to the development or progression of disease.

Retinoid signalling participates in the expression of ECM proteins, both directly (acting on their gene promoters) and indirectly (modifying the expression of profibrotic factors), and also affects the expression of cell membrane ECM receptors. Consequently, altered retinoid signalling induces changes in ECM/BM ultrastructure which are associated with fibrogenic activation in different organs and deterioration of tissue parenchyma. This can contribute to the disorders induced by VAD in organs and tissues.

Timoneda et al. (supra) report that, in an experimental model of chronic VAD rats, a thickening of the alveolar BM with an increase in the total amount of both type I and type IV collagens and a deposition of ectopic collagen fibrils in the BM was observed. The authors note that the mechanism through which VAD alters ECM is not clearly established, but explain that: “an alteration of the TGF-β1/Smad3 signalling pathway has been considered to play a central role and is associated with lung fibrosis. Among the different possibilities suggested, an alteration in the transforming growth factor-β1 (TGF-β1)/Smad3 signalling pathway has been considered to play a central role, and is associated with lung fibrosis. TGF-β1 via the Smad signalling pathway can upregulate the expression of several collagens, and also via non-Smad signalling can activate the expression of other ECM molecules and its composition. Additionally, TGF-β1 is an inducer of EMT in alveolar epithelial cells, which has been suggested as an early event in the development of pulmonary fibrosis. Moreover, TGF-β1, via an integration of the Smad3 and STAT3 signalling pathways stimulates the connective tissue growth factor (CTGF), a central mediator of ECM production. In agreement, increased levels of TGF-31 have been found in VAD tissues such as kidney, lung and aorta.”

Although Timoneda et al report that most of the VAD-induced alterations of ECM are reversed by RA, and that RA exhibits anti-proliferative, anti-inflammatory, anti-migratory and anti-fibrogenic activities and ameliorates bleomycin-induced lung fibrosis by downregulating the TGF-β1/Smad3 signalling pathway in rats, there is no suggestion in this document regarding an effective treatment of pulmonary fibrosis in subjects that do not have artificially induced lung fibrosis (i.e. bleomycin-induced lung fibrosis in rats), human subjects, or subjects who are not vitamin A deficient.

There is an urgent need, therefore, to provide effective therapies for pulmonary fibrosis.

The applicant has recognised that vitamin A may be used to inhibit scar tissue formation in the lungs, thereby reducing damage caused to the lungs as a result of fibrosis and potentially allowing enhanced replacement of injured cells by cells of the same type in the regenerative phase. Vitamin A may thus be used in the effective prevention, treatment, or amelioration of pulmonary fibrosis.

According to the invention there is provided vitamin A for use in the prevention, treatment, or amelioration of pulmonary fibrosis.

There is also provided according to the invention use of vitamin A in the manufacture of a medicament for the prevention, treatment, or amelioration of pulmonary fibrosis.

There is further provided according to the invention a method of preventing, treating, or ameliorating pulmonary fibrosis in a subject in need thereof, which comprises administering to the subject an effective amount of vitamin A.

In particular, vitamin A is able to prevent, treat, or ameliorate pulmonary fibrosis by inhibiting formation of pulmonary scar tissue.

Optionally the vitamin A inhibits scar tissue formation by anti-inflammatory action.

Optionally the vitamin A inhibits scar tissue formation by increasing expression of collagenase.

Vitamin A may cause a reduction in the amount of scar tissue already formed after an injury has occurred.

Optionally the scar tissue is actively maintained scar tissue.

It will be appreciated that use of a natural vitamin for the prevention, treatment, or amelioration of pulmonary fibrosis is particularly advantageous because of its known safety profile.

The pulmonary fibrosis may be a secondary effect of any of the following:
an infection, including a viral infection, or a bacterial infection (such as tuberculosis)
an autoimmune disease
a connective tissue disease, such as rheumatoid arthritis, ankylosing spondylitis, systemic lupus erythematosus (SLE), or scleroderma
a disease involving connective tissue, such as sarcoidosis and granulomatosis with polyangiitis
administration of a medication, for example amiodarone, bleomycin (pingyangmycin), busulfan, methotrexate, apomorphine, and nitrofurantoin
radiation therapy to the chest
inhalation of an environmental or occupational pollutant, such as metals in asbestosis, silicosis or exposure to certain gases.
hypersensitivity pneumonitis, most often resulting from inhaling dust contaminated with bacterial, fungal, or animal products

Optionally the pulmonary fibrosis is associated with a pulmonary infection. The pulmonary infection may be a viral, bacterial, or fungal pulmonary infection. Optionally the pulmonary infection is a chronic pulmonary infection.

The most common symptom of a chronic pulmonary infection is a persistent, severe cough (for example, lasting for more than three weeks). The sufferer will often bring up phlegm or mucus when coughing, and in the most severe cases, blood. Some patients with chronic pulmonary infections also experience some or all of the following symptoms (vary in severity from person to person): fever and sometimes sweats; a tight feeling across the chest, or sometimes sharp stabbing pain (pleurisy); shortness of breath which may involve wheezing; fatigue.

Examples of chronic pulmonary infections include: pneumonia, chronic bronchitis, influenza, the common cold, chronic sinusitis, rhinitis, Streptococcal pharyngitis, bronchiolitis, and bronchiectasis. Causative agents of pulmonary infections that may be associated with pulmonary fibrosis include: Bordetella pertussis (whooping cough), Influenza A virus (for example, swine flu (H1N1), bird flu (H5N1)), influenza B virus, enterovirus, SARS coronavirus (SARS-COV) (Severe acute respiratory syndrome, SARS), SARS coronavirus 2 (SARS-COV-2) (Coronavirus Disease 2019, COVID-19), MERS coronavirus (MERS-COV) (Middle East respiratory syndrome, MERS), Histoplasma capsulatum (Histoplasmosis), Mycobacterium tuberculosis (tuberculosis), Blastomyces dermatitidis (pulmonary blastomycosis).

Bacterial pneumonia is caused by Streptococcus pneumoniae, Haemophilus influenza, Chlamydophila pneumoniae, Mycoplasma pneumoniae; Staphylococcus aureus; Moraxella catarrhalis; and Legionella pneumophila. Viral pneumonia is caused by respiratory syncytial virus, parainfluenza, adenovirus, rhinoviruses, coronaviruses, influenza virus, respiratory syncytial virus (RSV), adenovirus, and parainfluenza. Fungal pneumonia is caused by Histoplasma capsulatum, Blastomyces, Cryptococcus neoformans, Pneumocystis jiroveci (pneumocystis pneumonia, or PCP), and Coccidioides immitis.

Viruses cause most cases of bronchitis and bronchiolitis. In community-acquired pneumonias, the most common bacterial agent is Streptococcus pneumoniae. Atypical pneumonias are cause by such agents as Mycoplasma pneumoniae, Chlamydia spp, Legionella, Coxiella burnetti and viruses. Nosocomial pneumonias and pneumonias in immunosuppressed patients have protean etiology with gram-negative organisms and staphylococci as predominant organisms.

Bronchiectasis may be associated with a range of bacterial, mycobacterial, and viral lung infections. Bacterial infections commonly associated with bronchiectasis include P. aeruginosa, H. influenzae, and S. pneumoniae. Nontuberculous mycobacteria infections such as Mycobacterium avium complex, and Nocardia infections have also been implicated.

Optionally the pulmonary infection is a viral pulmonary infection. Optionally the viral pulmonary infection is an Epstein-Barr virus (EBV), cytomegalovirus (CMV), human herpesvirus 7 (HHV-7), or human herpesvirus 8 (HHV-8) infection. Optionally the viral pulmonary infection is a coronavirus pulmonary infection, such as a severe acute respiratory syndrome coronavirus 2 (SARS-COV-2) pulmonary infection.

Optionally the pulmonary fibrosis is idiopathic pulmonary fibrosis (IPF).

The term “pulmonary fibrosis” is used herein to include pulmonary fibrosis occurring in interstitial lung disease. Interstitial lung disease (ILD), or diffuse parenchymal lung disease (DPLD), is a group of lung diseases affecting the interstitium (the tissue and space around the alveoli (air sacs of the lungs). It concerns alveolar epithelium, pulmonary capillary endothelium, basement membrane, and perivascular and perilymphatic tissues. It may occur when an injury to the lungs triggers an abnormal healing response. Ordinarily, the body generates just the right amount of tissue to repair damage, but in interstitial lung disease, the repair process goes awry and the tissue around the air sacs (alveoli) becomes scarred and thickened. This makes it more difficult for oxygen to pass into the bloodstream. The average rate of survival for someone with this disease is currently between 3 and 5 years. Prolonged ILD may result in pulmonary fibrosis.

Idiopathic interstitial pneumonia is the term given to ILDs with an unknown cause. They represent the majority of cases of interstitial lung diseases (up to two-thirds of cases). They were subclassified by the American Thoracic Society in 2002 into 7 subgroups: Idiopathic pulmonary fibrosis (IPF): the most common subgroup; Desquamative interstitial pneumonia (DIP); Acute interstitial pneumonia (AIP): also known as Hamman-Rich syndrome; Nonspecific interstitial pneumonia (NSIP); Respiratory bronchiolitis-associated interstitial lung disease (RB-ILD); Cryptogenic organizing pneumonia (COP): also known as Bronchiolitis Obliterans Organizing Pneumonia (BOOP); Lymphoid interstitial pneumonia (LIP).

Secondary ILDs are those diseases with a known etiology, including:

### Connective Tissue and Autoimmune Diseases:

Sarcoidosis;
Rheumatoid; arthritis;
Systemic lupus erythematosus;
Systemic sclerosis;
Polymyositis;
Dermatomyositis;
Antisynthetase syndrome

### Inhaled Substances:

### Inorganic

Silicosis
Asbestosis
Berylliosis
Industrial printing chemicals (e.g. carbon black, ink mist)

### Organic

Hypersensitivity pneumonitis (Extrinisic allergic alveolitis) Drug-induced:
Antibiotics
Chemotherapeutic drugs
Antiarrhythmic agents

### Infection:

Coronavirus disease 2019 (COVID-19)
Atypical pneumonia
Pneumocystis pneumonia (PCP)
Tuberculosis
Chlamydia trachomatis
Respiratory Syncytial Virus Malignancy:
Lymphangitic carcinomatosis

### Predominately in Children:

Diffuse developmental disorders
Growth abnormalities deficient alveolarisation
Infant conditions of undefined cause
ILD related to alveolar surfactant region

Vitamin A is the name of a group of fat-soluble retinoids, including retinol, retinal, and retinyl esters. There are two different categories of vitamin A. The first category, preformed vitamin A, comprises retinol and its esterified form, retinyl ester. The second category, provitamin A, comprises provitamin A carotenoids such as alpha-carotene, beta-carotene and beta-cryptoxanthin. Both retinyl esters and provitamin A carotenoids are converted to retinol, which is oxidized to retinal and then to retinoic acid. Both provitamin A and preformed vitamin A are known be metabolized intracellularly to retinal and retinoic acid, the bioactive forms of vitamin A.

Vitamin A for use according to the invention may be an isolated form of vitamin A. An isolated form of vitamin A is any form of vitamin A found in the diet or a metabolized form thereof. For example, vitamin A may be isolated from fish liver oil. Vitamin A may comprise a preformed vitamin A such as retinol or a retinyl ester. Retinyl esters include retinyl acetate and retinyl palmitate. Vitamin A may comprise a provitamin A, such as a provitamin A carotenoid including alpha-carotene, beta-carotene or beta-cryptoxanthin. Vitamin A may comprise a bioactive form of vitamin A such as retinal or retinoic acid.

Vitamin A is available for human consumption in multivitamins and as a stand-alone supplement, often in the form of retinyl acetate or retinyl palmitate. A portion of the vitamin A in some supplements is in the form of beta-carotene and the remainder is preformed vitamin A; others contain only preformed vitamin A or only beta-carotene. Supplement labels usually indicate the percentage of each form of the vitamin. The amounts of vitamin A in stand-alone supplements range widely. Multivitamin supplements typically contain 2,500 to 10,000 international units (IU) vitamin A, often in the form of both retinol and beta-carotene.

Vitamin A is listed on food and supplement labels in international units (IUs). However, Recommended Dietary Allowance (RDA) (average daily level of intake sufficient to meet the nutrient requirements of nearly all (97%-98%) healthy individuals) for vitamin A is given as micrograms (μg; mcg) of retinol activity equivalents (RAE) to account for the different bioactivities of retinol and provitamin A carotenoids (see Table 1). Because the body converts all dietary sources of vitamin A into retinol, 1 mcg of physiologically available retinol is equivalent to the following amounts from dietary sources: 1 mcg of retinol, 12 mcg of beta-carotene, and 24 mcg of alpha-carotene or beta-cryptoxanthin. From dietary supplements, the body converts 2 mcg of beta-carotene to 1 mcg of retinol.

Conversion rates between mcg RAE and IU are as follows:
1 IU retinol=0.3 mcg RAE;
1 IU beta-carotene from dietary supplements=0.15 mcg RAE;
1 IU beta-carotene from food=0.05 mcg RAE; and
1 IU alpha-carotene or beta-cryptoxanthin=0.025 mcg RAE.

An RAE cannot be directly converted into an IU without knowing the source(s) of vitamin A. For example, the RDA of 900 mcg RAE for adolescent and adult men is equivalent to 3,000 IU if the food or supplement source is preformed vitamin A (retinol). However, this RDA is also equivalent to 6,000 IU of beta-carotene from supplements, 18,000 IU of beta-carotene from food, or 36,000 IU of alpha-carotene or beta-cryptoxanthin from food. So a mixed diet containing 900 mcg RAE provides between 3,000 and 36,000 IU of vitamin A, depending on the foods consumed.

TABLE 1
Recommended Dietary Allowances (RDAs) for Vitamin A

| Age | | Male | Female | Pregnancy | Lactation |
|---|---|---|---|---|---|
| 0-6 | months* | 400 mcg RAE | 400 mcg RAE | | |
| 7-12 | months* | 500 mcg RAE | 500 mcg RAE | | |
| 1-3 | years | 300 mcg RAE | 300 mcg RAE | | |
| 4-8 | years | 400 mcg RAE | 400 mcg RAE | | |
| 9-13 | years | 600 mcg RAE | 600 mcg RAE | | |
| 14-18 | years | 900 mcg RAE | 700 mcg RAE | 750 mcg RAE | 1,200 mcg RAE |
| 19-50 | years | 900 mcg RAE | 700 mcg RAE | 770 mcg RAE | 1,300 mcg RAE |
| 51+ | years | 900 mcg RAE | 700 mcg RAE | | |

*Adequate Intake (AI), equivalent to the mean intake of vitamin A in healthy, breastfed infants.
Source: National Institutes of Health, Vitamin A, Fact Sheet for Health Professionals, as updated 5 Oct. 2018

The Food and Nutrition Board (FNB) at the Institute of Medicine of the National Academies (formerly National Academy of Sciences) has established tolerable Upper Intake Level (UL) (maximum daily intake unlikely to cause adverse health effects) for preformed vitamin A that apply to both food and supplement intakes. The FNB based these ULs on the amounts associated with an increased risk of liver abnormalities in men and women, teratogenic effects, and a range of toxic effects in infants and children. The FNB has not established ULs for beta-carotene and other provitamin A carotenoids.

TABLE 2
Tolerable Upper Intake Levels (ULs) for Preformed Vitamin A*

| Age | | Male | Female | Pregnancy | Lactation |
|---|---|---|---|---|---|
| 0-12 | months | 600 mcg RAE | 600 mcg RAE | | |
| | | (2,000 IU) | (2,000 IU) | | |
| 1-3 | years | 600 mcg RAE | 600 mcg RAE | | |
| | | (2,000 IU) | (2,000 IU) | | |
| 4-8 | years | 900 mcg RAE | 900 mcg RAE | | |
| | | (3,000 IU) | (3,000 IU) | | |
| 9-13 | years | 1,700 mcg RAE | 1,700 mcg RAE | | |
| | | (5,667 IU) | (5,667 IU) | | |
| 14-18 | years | 2,800 mcg RAE | 2,800 mcg RAE | 2,800 mcg RAE | 2,800 mcg RAE |
| | | (9,333 IU) | (9,333 IU) | (9,333 IU) | (9,333 IU) |
| 19+ | years | 3,000 mcg RAE | 3,000 mcg RAE | 3,000 mcg RAE | 3,000 mcg RAE |
| | | (10,000 IU) | (10,000 IU) | (10,000 IU) | (10,000 IU) |

Source: National Institutes of Health, Vitamin A, Fact Sheet for Health Professionals, as updated 5 Oct. 2018
*These ULs, expressed in mcg and in IUs (where 1 mcg = 3.33 IU), only apply to products from animal sources and supplements whose vitamin A comes entirely from retinol or ester forms, such as retinyl palmitate. However, many dietary supplements (such as multivitamins) do not provide all of their vitamin A as retinol or its ester forms. For example, the vitamin A in some supplements consists partly or entirely of beta-carotene or other provitamin A carotenoids. In such cases, the percentage of retinol or retinyl ester in the supplement should be used to determine whether an individual's vitamin A intake exceeds the UL. For example, a supplement labeled as containing 10,000 IU of vitamin A with 60% from beta-carotene (and therefore 40% from retinol or retinyl ester) provides 4,000 IU of preformed vitamin A. That amount is above the UL for children from birth to 13 years but below the UL for adolescents and adults.

The Vitamin A may be provided from a mixture of different sources, including, for example, in normal feed, and in the form of a supplement.

Optionally vitamin A for use in the prevention, treatment, or amelioration of pulmonary fibrosis in a subject according to the invention comprises a high dose of vitamin A.

A high dose of vitamin A is considered to be a dose that exceeds a UL for the subject. Examples of high doses of vitamin A include: >10,000 IU to 100,000 IU vitamin A per day; about 25,000 to 50,000 IU per day; about 25,000 to 75,000 IU vitamin A per day; about 25,000 to 100,000 IU vitamin A per day; about 50,000 to 100,000 IU vitamin A per day; or about 75,000 to 100,000 IU vitamin A per day, in particular of preformed vitamin A.

A high dose of vitamin A may be a dose that is 5%-50% of a minimum toxic dose for the subject.

Vitamin A may be administered to the subject at least once per day.

Vitamin A may be administered to the subject once per day, twice per day, three times per day, four times per day, or five times per day.

Vitamin A may be administered to the subject for at least 3 days for example for at least a week, for at least a month, or for at least 6 months from the day of first administration to the subject.

Vitamin A may be administered to the subject for at least five weeks from the day of first administration to the subject.

Vitamin A may be administered to the subject for at least five weeks from the day of first administration to the subject for treatment of an injury that occurred over six months prior to the date of first administration of Vitamin A to the subject in accordance with the invention.

Prolonged exposure to high doses of vitamin A may lead to hypervitaminosis A. Thus, it may be preferred to limit administration of high doses of vitamin A to the subject for up to 6 years, or up to 6 months, from the day of first administration to the subject.

Optionally for an adult human subject (>18 years old), the subject may be administered up to 100,000 IU vitamin A (in particular of preformed vitamin A) per day for up to 6 months. For example, >10,000 IU to 100,000 IU vitamin A per day; about 25,000 to 50,000 IU per day; about 25,000 to 75,000 IU vitamin A per day; about 25,000 to 100,000 IU vitamin A per day; about 50,000 to 100,000 IU vitamin A per day; or about 75,000 to 100,000 IU vitamin A per day (in particular of preformed vitamin A) for up to 6 months.

Optionally for an adult human subject (>18 years old), the subject may be administered up to 25,000 IU vitamin A per day (in particular of preformed vitamin A) for up to 6 years. For example, >10,000 IU to 25,000 IU vitamin A per day (in particular of preformed vitamin A) for up to 6 years

Optionally the subject is administered up to 50% (for example >10% to 50%, or 25% to 50%) of a maximum safe dose of vitamin A (in particular of preformed vitamin A) for the subject per day.

For example, a maximum safe dose of vitamin A (in particular of preformed vitamin A) per day for an adult human subject may be 100,000 IU vitamin A (in particular of preformed vitamin A).

Optionally the subject may be administered a dose of vitamin A which is upto 50% of a minimum toxic dose for the subject.

Optionally the subject may be administered a dose of vitamin A which is at least 5% of a minimum toxic dose for the subject.

For example, a minimum toxic dose for a subject may be 1,000 IU/kg body weight (BW)/day.

Optionally the subject is a mammalian subject. Optionally the subject is not a rat. Optionally the subject is a human subject.

Optionally the subject is not vitamin A deficient.

Optionally the subject is vitamin A deficient.

Plasma retinol levels are typically measured to assess vitamin A status. However, plasma retinol levels are under tight hepatic homeostatic control and do not decline until vitamin A concentration in the liver is almost depleted (critical liver concentration≤20 μg g-1 of liver). Liver vitamin A reserves can be measured indirectly through the relative dose-response test (McLaren, D. S.; Kraemer, K. Manual on Vitamin Deficiency Disorders (VADD), 3rd ed.; Sight and Life Press:Basel, Switzerland, 2012; ISBN 978-3-906412-58-0), which is considered the “gold standard” indicator of whole-body vitamin A status. However, for clinical purposes, plasma retinol levels alone are sufficient and commonly used for documenting significant deficiency of vitamin A. The physiological plasma concentration of vitamin A is 1-2 μmol/L and, according to the World Health Organization, values of serum retinol concentrations below a cut-off of 0.70 μmol/L (or 20 μg/dL) represent biochemical vitamin A deficiency (VAD), and values lower than 0.35 μmol/L are indicative of severe deficiency and associated with numerous clinical manifestations.

Optionally the subject has a serum retinol concentration of at least 0.7 μmol/L.

Optionally the subject has a plasma concentration of vitamin A of 1-2 μmol/L.

Optionally the vitamin A is to be administered to the subject at a dose that results in a plasma concentration of vitamin A in excess of 2 μmol/L.

Optionally the subject has not been administered bleomycin.

Optionally the subject does not have bleomycin-induced lung fibrosis.

It will be appreciated that vitamin A should preferably be administered to the subject as soon as possible after the subject has been diagnosed as:
being at risk of developing pulmonary fibrosis;
having pulmonary fibrosis; or
having a disease or condition (for example, an ILD) in which pulmonary fibrosis is a secondary effect.

Optionally vitamin A is administered within a month, within a week, or within a day of the diagnosis. Optionally vitamin A is administered with a week of the diagnosis.

Beneficial effects of treatment with vitamin A may also be observed when treatment is initiated many months or even years after diagnosis. Thus, optionally vitamin A may be administered months, years or even decades after diagnosis, for example within six months, a year, or a decade, or within twenty, thirty, forty, fifty, or sixty years of the diagnosis.

Pulmonary fibrosis may be diagnosed using any suitable techniques known to the skilled person. Examples (which may be used alone or in combination) include: X-rays; high-resolution computed tomography (HRCT) scans; semi-quantitative computed tomography (the most pertinent computed tomography patterns of fibrotic ILD are reticulation, traction, bronchiectasis, honeycombing and ground-glass opacification); computed tomography-derived quantitative lung fibrosis measures (quantitative CT); clinical markers (for example, dyspnoea and cough); physiological markers (for example, forced vital capacity (FVC) and, to a lesser extent, diffusing capacity of the lung for carbon monoxide (DLCO)); lung biopsy (usually by a small incision through the ribs with a thoracoscope); serum biomarkers; a pulmonary function test (using a device to measure breathing capacity), an oxygen desaturation study (the patient walks for almost 6 minutes while their oxygen level is measured through a probe attached to the finger or the forehead), other laboratory tests (for example, to rule out other diseases), including autoantibody tests, full blood count, electrolytes, creatinine levels, liver function tests, arterial blood gas.

Diagnosis of IPF is discussed in: Christe et al. (“Computer-Aided Diagnosis of Pulmonary Fibrosis Using Deep Learning and CT Images”, Invest Radiol. 2019 October; 54(10): 627-632); Robbie et al., (“Evaluating disease severity in idiopathic pulmonary fibrosis”, Eur Respir Rev 2017; 26: 170051); Martinez et al. (“The diagnosis of idiopathic pulmonary fibrosis: current and future approaches”, Lancet Respir Med. 2017 January; 5(1): 61-71); Raghu et al. (“Diagnosis of idiopathic pulmonary fibrosis. An official ATS/ERS/JRS/ALAT clinical practice guideline”, Am J Respir Crit Care Med. 2018 Sep. 1; 198(5): e44-e68); Nakamura and Suda (“Idiopathic Pulmonary Fibrosis: Diagnosis and Clinical Manifestations”, Clin Med Insights Circ Respir Pulm Med. 2015; 9(Suppl 1): 163-171); Lynch et al. (“High-Resolution Computed Tomography in Idiopathic Pulmonary Fibrosis”, Am J Respir Crit Care Med, Vol 172. pp 488-493, 2005).

It may be determined whether administration of vitamin A according to the invention has prevented, treated, or ameliorated pulmonary fibrosis in a subject by any technique known to the skilled person. Examples of suitable techniques (which may be used alone or in combination) include those referred to above, in particular X-rays, HRCT scans, semi-quantitative CT, quantitative CT, clinical markers, physiological markers, serum biomarkers, a lung biopsy, a pulmonary function test, or an oxygen desaturation study. Serum biomarkers, semi-quantitative CT, and/or quantitative CT may be particularly useful measures (see Robbie et al., 2017).

Effective treatment or amelioration of pulmonary fibrosis may include any slowing of the rate of progression of pulmonary fibrosis observed in the subject prior to administration of vitamin A (including, for example, any slowing of the rate of formation of pulmonary scar tissue), or any slowing of the rate of deterioration of lung function observed in the subject prior to administration of vitamin A.

The vitamin A may be administered to a subject by any suitable route. Examples include systemic administration, for example orally or intravenously. Optionally the vitamin A is administered to a subject by inhalation.

There is also provided according to the invention a pharmaceutical composition for oral administration, which comprises Vitamin A and a pharmaceutically acceptable plant oil. Optionally the plant oil comprises a coconut oil.

Optionally a pharmaceutical composition of the invention comprises a unit dose of vitamin A, wherein the unit dose comprises up to 100,000 IU vitamin A, for example >10,000 IU to 100,000 IU vitamin A; about 25,000 to 50,000 IU per day; about 25,000 to 75,000 IU vitamin A per day; 25,000 to 100,000 IU vitamin A; 50,000 to 100,000 IU vitamin A; or 75,000 to 100,000 IU vitamin A (in particular of preformed vitamin A).

There is also provided according to the invention a sterile sachet comprising a pharmaceutical composition of the invention.

There is further provided according to the invention a package comprising a plurality of separate unit doses of vitamin A, wherein each unit dose comprises a pharmaceutical composition of the invention.

A package of the invention may comprise at least 7 unit doses, at least 30 unit doses, or at least 100 unit doses of vitamin A.

Vitamin A of a pharmaceutical composition of the invention may comprise any combination of vitamin A described previously.

There is also provided according to the invention a pharmaceutical composition of the invention for use in prevention, treatment, or amelioration of pulmonary fibrosis in a subject.

Use of vitamin A in accordance with the invention may be particularly effective for the treatment of older subjects. For example, a human subject may be at least 18 years old, at least 25 years old, at least 30 years old, at least 40 years old, or at least 50 years old.

### Maintenance Doses

We have found that administering a post-treatment maintenance dose of vitamin A supplement provides continued benefits. As described in more detail in Example 10 below, after several weeks of treatment with “full-treatment doses” of vitamin A supplement, horses with connective tissue injuries (tendon or ligament) were orally administered vitamin A once per day with a reduced, “maintenance dose” of half the “full-treatment dose” of vitamin A supplement for 7 weeks. The results provide evidence for beneficial effects of continuing with a maintenance dose of vitamin A supplement after a period of administration of full-treatment doses.

Accordingly, after a period of treatment of a subject with “full-treatment doses” of Vitamin A, it may be advantageous to continue treatment for a further period with “maintenance doses” of Vitamin A.

A “maintenance dose” is a dose of vitamin A, or of a composition comprising vitamin A, which is less than a “full-treatment dose”.

Optionally a maintenance dose is up to three quarters of a full-treatment dose, or up to two-thirds of a full treatment dose.

Optionally a maintenance dose is at least a quarter of a full-treatment dose.

Optionally a maintenance dose is to be administered after the subject has been administered one or more full-treatment doses.

Optionally a maintenance dose is to be administered from the day after the last administration of a full-treatment dose.

Typically, a plurality of maintenance doses is to be administered to the subject.

Optionally the maintenance doses are to be administered for at least four weeks from the day of first administration of a maintenance dose to the subject.

Optionally the maintenance doses are to be administered for at least 12 weeks from the day of first administration of a maintenance dose to the subject.

Optionally the maintenance doses are to be administered for at least 6 months from the day of first administration of a maintenance dose to the subject.

Optionally the maintenance doses are to be administered for at least 12 months from the day of first administration of a maintenance dose to the subject.

Optionally the maintenance doses are to be administered for up to 6 years from the day of first administration of a maintenance dose to the subject.

Optionally the subject is a human subject.

For a human subject the, or each full-treatment dose optionally comprises >10,000 to 100,000 IU vitamin A per day.

For a human subject the, or each full-treatment dose optionally comprises about 25,000-100,000, 50,000-100,000, or 75,000-100,000 IU vitamin A per day

For a human subject the, or each maintenance dose optionally comprises >2,500 IU to 75,000 IU vitamin A per day.

For a human subject optionally the, or each maintenance dose comprises 5,000-75,000, 10,000-75,000, or 20,000-75,000 IU vitamin A per day.

For a human subject optionally the, or each maintenance dose comprises 5,000-50,000, 10,000-50,000, or 20,000-50,000 IU vitamin A per day.

A “maintenance dose” may be administered as a single dose, or in multiple dose units. For example, a maintenance dose of 6,000 IU vitamin A per day may be provided as two doses of 3,000 IU vitamin A, one dose to be given in the morning, and another dose to be given in the evening.

Similarly, a “full-treatment dose” may be administered as a single dose, or in multiple dose units. For example, a full-treatment dose of 12,000 IU vitamin A per day may be provided as two doses of 6,000 IU vitamin A, one dose to be given in the morning, and another dose to be given in the evening.

There is also provided according to the invention a multiple-dose formulation which comprises a plurality of separate unit doses of vitamin A, wherein each unit dose is a maintenance dose of vitamin A, and wherein each unit dose comprises >2,500 IU to 75,000 IU vitamin A.

Optionally the, or each unit dose comprises 5,000-75,000, 10,000-75,000, or 20,000-75,000 IU vitamin A.

Optionally the, or each unit dose comprises 5,000-50,000, 10,000-50,000, or 20,000-50,000 IU vitamin A.

Optionally the, or each unit dose of a multiple-dose formulation of the invention is for administration to a human subject.

Optionally the vitamin A comprises isolated vitamin A.

Optionally the vitamin A comprises a preformed vitamin A, such as a retinyl ester or retinol.

Optionally the vitamin A comprises a provitamin A, such as a carotenoid.

Optionally the vitamin A comprises a bioactive form of vitamin A, such as retinal or retinoic acid.

Optionally the vitamin A is part of a pharmaceutical composition comprising vitamin A and a pharmaceutically acceptable carrier, excipient or diluent.

Optionally the composition is a sterile composition.

Optionally the vitamin A is the only non-cellular, non-antibiotic, active agent present in the pharmaceutical composition.

Optionally a multiple-dose formulation of the invention comprises at least 7, at least 30, or at least 100 separate unit doses of vitamin A.

There is also provided according to the invention a multiple-dose formulation which comprises: a first plurality of separate unit doses of vitamin A, wherein each unit dose of the first plurality of separate unit doses is a full-treatment unit dose of vitamin A; and a second plurality of separate unit doses of vitamin A, wherein each unit dose of the second plurality of separate unit doses is a maintenance dose of vitamin A.

A multiple-dose formulation of the invention comprising a first plurality of separate unit doses and a second plurality of unit doses may be for treatment of a human subject.

For a human subject optionally each maintenance unit dose comprises >2,500 IU to 75,000 IU vitamin A.

For a human subject optionally each maintenance unit dose comprises 5,000-75,000, 10,000-75,000, or 20,000-75,000 IU vitamin A.

For a human subject optionally each maintenance unit dose comprises 5,000-50,000, 10,000-50,000, or 20,000-50,000 IU vitamin A.

For a human subject optionally each full treatment unit dose comprises >10,000 IU to 100,000 IU vitamin A.

For a human subject optionally each full treatment unit dose comprises 25,000-100,000, 50,000-100,000, or 75,000-100,000 IU vitamin A.

The term “unit dose” as used herein refers to physically discrete units suited as unitary doses for the subject to be treated. That is, the vitamin A (or composition comprising vitamin A) is formulated into discrete dose units each containing a predetermined “unit dose” quantity of vitamin A calculated to produce the desired therapeutic effect, typically in association with a required pharmaceutical carrier, excipient or diluent. It should be noted that, in some cases, two or more individual dose units in combination provide a therapeutically effective amount of the active ingredient, for example, two tablets or capsules taken together (or sequentially) may provide a therapeutically effective dose, such that the unit dose in each tablet or capsule is approximately 50% of the therapeutically effective amount.

Each unit dose of a multiple-dose formulation of the invention is typically provided as a sterile unit dose.

A multiple-dose formulation of the invention may be provided packaged in a container. The container can be, for example, a bottle (e.g., with a closure device, such as a cap), a blister pack (e.g., which can provide for enclosure of one or more doses per blister), a vial, flexible packaging (e.g., sealed Mylar or plastic bags), an ampule (for single doses in solution), a dropper, a syringe, thin film, a tube and the like. In some embodiments, a container, such as a sterile container, comprises a subject pharmaceutical composition. In some embodiments the container is a bottle or a syringe. In some embodiments the container is a bottle. In some embodiments the container is a syringe.

For example, each unit dose of the multiple-dose formulation may be provided in a separate well or blister of the container, with a foil seal covering each well/blister. In addition to the container containing the unit doses of the multiple-dose formulation, an information package insert may be included describing the use and attendant benefits of the active ingredient (for example, vitamin A or a composition comprising vitamin A) in treating the condition of interest (for example, tissue damage). Preferred compounds, compositions, and unit doses are those described herein.

There is also provided according to the invention a multiple-dose formulation of the invention for use as a medicament.

There is also provide according to the invention a multiple-dose formulation of the invention for use in the prevention, treatment, or amelioration of pulmonary fibrosis.

There is further provided according to the invention use of a multiple-dose formulation of the invention in the manufacture of a medicament for the prevention, treatment, or amelioration of pulmonary fibrosis.

Optionally the multiple-dose formulation inhibits formation of pulmonary scar tissue.

Optionally the pulmonary fibrosis is associated with a pulmonary infection.

Optionally the pulmonary infection is a viral pulmonary infection.

Optionally the viral pulmonary infection is a severe acute respiratory syndrome coronavirus 2 (SARS-CoV-2) pulmonary infection.

Optionally the vitamin A comprises isolated vitamin A.

Optionally the vitamin A comprises a preformed vitamin A, such as a retinyl ester or retinol.

Optionally the vitamin A comprises a provitamin A, such as a carotenoid.

Optionally the vitamin A comprises a bioactive form of vitamin A, such as retinal or retinoic acid.

Optionally a multiple-dose formulation of the invention is for administration to a human subject.

Optionally in a method of the invention the subject is administered a maintenance dose of vitamin A, wherein the maintenance dose is less than a full treatment dose.

Optionally the maintenance dose is up to three quarters of a full treatment dose.

Optionally the maintenance dose is up to two-thirds of a full treatment dose.

Optionally the maintenance dose is at least a quarter of a full treatment dose.

Optionally the maintenance dose is administered after the subject has been administered one or more full-treatment doses.

Optionally the maintenance dose is administered from the day after the last administration of a full-treatment dose to the subject.

Optionally a plurality of maintenance doses administered to the subject.

Optionally the maintenance doses are administered for at least four weeks from the day of first administration of a maintenance dose to the subject.

Optionally the maintenance doses are administered for at least 12 weeks from the day of first administration of a maintenance dose to the subject.

Optionally the maintenance doses are administered for at least 6 months from the day of first administration of a maintenance dose to the subject.

Optionally the maintenance doses are administered for at least 12 months from the day of first administration of a maintenance dose to the subject.

Optionally the maintenance doses are administered for up to 6 years from the day of first administration of a maintenance dose to the subject.

Optionally the subject is a human subject.

Optionally the, or each maintenance dose comprises >2,500 IU to 75,000 IU vitamin A per day.

Optionally the, or each maintenance dose comprises 5,000-75,000, 10,000-75,000, or 20,000-75,000 IU vitamin A per day.

Optionally the, or each maintenance dose comprises 5,000-50,000, 10,000-50,000, or 20,000-50,000 IU vitamin A per day.

The vitamin A can be incorporated into a variety of formulations for therapeutic administration, more particularly by combination with appropriate, pharmaceutically acceptable carriers, pharmaceutically acceptable diluents, or other pharmaceutically acceptable excipients, and can be formulated into preparations in solid, semi-solid, liquid or gaseous forms, such as tablets, capsules, powders, granules, ointments, solutions, suppositories, injections, inhalants and aerosols as appropriate.

Optionally the vitamin A is in solid form. Optionally the vitamin A is not in an organic solution. Optionally the vitamin A is not encapsulated by, or attached to a microparticle. Optionally the vitamin A is not encapsulated by, or attached to a nanoparticle.

Vitamin A can be administered in the form of a pharmaceutically acceptable salt. It can also be used alone or in appropriate association, as well as in combination, with other pharmaceutically active compounds. Optionally vitamin A is administered with an antibiotic agent, an anti-viral agent, or an anti-fungal agent. Optionally vitamin A is the only non-cellular, non-antibiotic, active agent administered.

The following methods and excipients are merely exemplary and are in no way limiting.

For oral preparations, vitamin A can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Vitamin A can be formulated into preparations for injection by dissolving, suspending or emulsifying in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, a pharmaceutical composition of the present disclosure can comprise further agents such as dopamine or psychopharmacologic drugs, depending on the intended use of the pharmaceutical composition.

Pharmaceutical compositions are prepared by mixing Vitamin A having the desired degree of purity with optional pharmaceutically acceptable carriers, other excipients, stabilizers, surfactants, buffers and/or tonicity agents. Acceptable carriers, other excipients and/or stabilizers are nontoxic to recipients at the dosages and concentrations employed, and may include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54. An aqueous formulation can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the formulation to reduce aggregation and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant can range from about 0.001% to about 1% w/v.

A lyoprotectant can also be added in order to protect a labile active ingredient against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Unit dosage forms for oral administration such as syrups, elixirs, and suspensions can be provided wherein each dosage unit, for example, teaspoonful, tablespoonful, or tablet contains a predetermined amount of the active agent (i.e. vitamin A). Similarly, unit dosage forms for injection or intravenous administration can comprise vitamin A in a composition as a solution in sterile water, normal saline or another pharmaceutically acceptable carrier.

The term “unit dosage form,” (or “unit dose”), as used herein, refers to physically discrete units suitable as unitary dosages for human or animal subjects, each unit containing a predetermined quantity of vitamin A, calculated in an amount sufficient to produce the desired effect in association with a pharmaceutically acceptable diluent, carrier or vehicle.

Vitamin A can be administered as an injectable formulation. Typically, injectable compositions are prepared as liquid solutions or suspensions; solid forms suitable for solution in, or suspension in, liquid vehicles prior to injection can also be prepared.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985. The composition or formulation to be administered will, in any event, contain a quantity of vitamin A adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

There is also provided according to the invention a pharmaceutical composition for inhalation, which comprises Vitamin A and a pharmaceutically acceptable carrier, excipient, or diluent.

Inhalable formulations are well known to the skilled person. Suitable examples are described in Hadiwinoto et al., “A review on recent technologies for the manufacture of pulmonary drugs”, Therapeutic Delivery, Vol. 9, No. 1

The required aerosol size to deliver drugs to the whole lung involves an aerodynamic diameter <˜5 μm. For delivery to the alveolar epithelium, particles with an even smaller size, for example, aerodynamic diameter <˜3 μm, are required (Newman, “Drug delivery to the lungs: challenges and opportunities”, Ther. Deliv. (2017) 8(8), 647-661).

Nanoparticles, microparticles, liposomes, powder, and microemulsions are commonly employed drug delivery carriers for pulmonary delivery (see Thakur et al., (2020) Patented therapeutic drug delivery strategies for targeting pulmonary diseases, Expert Opinion on Therapeutic Patents, 30:5, 375-387).

Optionally an excipient for a pharmaceutical composition of the invention for inhalation is selected from the group consisting of sugars and saccharides, preferably inhalation grade lactose, preferably alpha monohydrate lactose in the form of crystalline lactose, milled lactose or micronized lactose.

Optionally a pharmaceutical composition of the invention for inhalation comprises particles having an aerodynamic diameter of 0.5 to 10 μm.

Inhaled drug delivery is achieved using four principal technologies: dry powder inhalers, metered-dose inhalers, nebulisers and liquid inhalers. Suitable devices for administration of a pharmaceutical composition of the invention for inhalation are well-known to the skilled person. Examples are described in the following publications:
Brunaugh A. D., Smyth H. D. C., Williams III R. O. (2019) Pulmonary Drug Delivery. In: Essential Pharmaceutics. AAPS Introductions in the Pharmaceutical Sciences. Springer, Cham, pp 163-181;
Lalan M., Tandel H., Lalani R., Patel V., Misra A. (2019) Inhalation Drug Therapy: Emerging Trends in Nasal and Pulmonary Drug Delivery. In: Misra A., Shahiwala A. (eds) Novel Drug Delivery Technologies. Springer, Singapore
Lexmond A., Forbes B. (2016) Drug Delivery Devices for Inhaled Medicines. In: Page C., Barnes P. (eds) Pharmacology and Therapeutics of Asthma and COPD. Handbook of Experimental Pharmacology, vol 237. Springer, Cham (pp 265-280);
Ibrahim et al., “Inhalation drug delivery devices: technology update”, Med Devices (Auckl). 2015; 8: 131-139.

Administration by inhalation may have advantages over systemic administration, including a more rapid onset of action, an increased therapeutic effect, and, depending on the agent inhaled, reduced systemic side effects since the required local concentration in the lungs can be obtained with a lower dose.

There is also provided according to the invention a dry powder inhaler, a metered-dose inhaler, a nebuliser, or a liquid inhaler, comprising a pharmaceutical composition of the invention for inhalation.

Ranges may be expressed herein as from “about” one particular value, and/or to another particular value. When such a range is expressed, another embodiment includes from the one particular value and/or to the other particular value. Similarly, when values are expressed as approximations, by the use of the antecedent “about”, it will be understood that the particular value forms another embodiment.

Wherever the term “vitamin A” is used herein this includes reference to “vitamin A, or a pharmaceutically acceptable salt thereof”.

Elements of the second aspect of the invention are defined by the following numbered paragraphs:

1. Vitamin A for use in the prevention, treatment, or amelioration of pulmonary fibrosis.

2. Use of vitamin A in the manufacture of a medicament for the prevention, treatment, or amelioration of pulmonary fibrosis.

3. Vitamin A for use according to paragraph 1, or use of vitamin A according to paragraph 2, wherein the vitamin A inhibits formation of pulmonary scar tissue.

4. Vitamin A, or use of vitamin A, according to any preceding paragraph, wherein the pulmonary fibrosis is associated with a pulmonary infection.

5. Vitamin A, or use of vitamin A, according to paragraph 4, wherein the pulmonary infection is a viral pulmonary infection.

6. Vitamin A, or use of vitamin A, according to paragraph 5, wherein the viral pulmonary infection is a severe acute respiratory syndrome coronavirus 2 (SARS-COV-2) pulmonary infection.

7. Vitamin A, or use of vitamin A, according to any preceding paragraph, wherein the vitamin A comprises isolated vitamin A.

8. Vitamin A for use, or use of vitamin A, according to any preceding paragraph, wherein the vitamin A comprises a preformed vitamin A, such as a retinyl ester or retinol.

9. Vitamin A for use, or use of vitamin A, according to any preceding paragraph wherein the vitamin A comprises a provitamin A, such as a carotenoid.

10. Vitamin A for use, or use of vitamin A, according to any preceding paragraph wherein the vitamin A comprises a bioactive form of vitamin A, such as retinal or retinoic acid.

11. Vitamin A for use, or use of vitamin A, according to any preceding paragraph for administration at a dose in excess of a Tolerable Upper Limit Intake Level (UL) for the subject.

12. Vitamin A for use, or use of vitamin A, according to any preceding paragraph wherein the subject is a human subject.

13. Vitamin A for use, or use of vitamin A, according to paragraph 12 for administration to the subject at a dose of >10,000 to 100,000 IU vitamin A per day.

14. Vitamin A for use, or use of vitamin A, according to paragraph 13 for administration to the subject at a dose of about 25,000-100,000, 50,000-100,000, or 75,000-100,000 IU vitamin A per day.

15. Vitamin A for use, or use of vitamin A, according to any preceding paragraph for administration to the subject once per day.

16. Vitamin A for use, or use of vitamin A, according to paragraph 15 for administration for at least 3 days from the day of first administration to the subject.

17. Vitamin A for use, or use of vitamin A, according to paragraph 15 for administration for at least a week, at least a month, or at least 6 months from the day of first administration to the subject.

18. Vitamin A for use, or use of vitamin A, according to any of paragraphs 15 to 17 for administration to the subject for up to 6 years from the day of first administration to the subject.

19. Vitamin A for use, or use of vitamin A, according to any preceding paragraph for administration systemically.

20. Vitamin A for use, or use of vitamin A, according to paragraph 19 for administration to the subject orally or intravenously.

21. Vitamin A for use, or use of vitamin A, according to any preceding paragraph for administration to the subject by inhalation.

22. Vitamin A for use, or use of vitamin A, according to any preceding paragraph wherein the subject is not vitamin A deficient.

23. Vitamin A for use, or use of vitamin A, according to any preceding paragraph, wherein the subject has a serum retinol concentration of at least 0.7 μmol/L.

24. Vitamin A for use, or use of vitamin A, according to any preceding paragraph, wherein the subject has a plasma concentration of vitamin A of 1-2 μmol/L.

25. Vitamin A for use, or use of vitamin A, according to any preceding paragraph for administration to the subject at a dose that results in plasma concentration of vitamin A in excess of 2 μmol/L.

26. A pharmaceutical composition for inhalation, which comprises Vitamin A and a pharmaceutically acceptable carrier, excipient, or diluent.

27. A pharmaceutical composition according to paragraph 26, wherein the excipients are selected from the group consisting of sugars and saccharides, preferably inhalation grade lactose, preferably alpha monohydrate lactose in the form of crystalline lactose, milled lactose or micronized lactose.

28. A pharmaceutical composition according to paragraph 26 or 27, which comprises particles having an aerodynamic diameter of 0.5 to 10 μm.

29. A dry powder inhaler, or a nebulizer, comprising a pharmaceutical composition according to any of paragraphs 26 to 28.

30. A pharmaceutical composition for oral administration, which comprises Vitamin A and a pharmaceutically acceptable plant oil.

31. A pharmaceutical composition according to paragraph 30, wherein the plant oil comprises a coconut oil.

32. A pharmaceutical composition according to paragraph 30 or 31, which comprises a unit dose of vitamin A, wherein the unit dose comprises >10,000 IU to 100,000 IU vitamin A.

33. A sterile sachet comprising a pharmaceutical composition according to any of paragraphs 30 to 32.

34. A package comprising a plurality of separate unit doses of vitamin A, wherein each unit dose comprises a pharmaceutical composition according to any of paragraphs 30 to 32.

35. A method of preventing, treating, or ameliorating pulmonary fibrosis in a subject in need thereof, which comprises administering to the subject an effective amount of vitamin A.

36. A method according to paragraph 35, wherein the vitamin A inhibits formation of pulmonary scar tissue.

37. A method according to paragraph 35 or 36, wherein the pulmonary fibrosis is associated with a pulmonary infection.

38. A method according to paragraph 37, wherein the pulmonary infection is a viral pulmonary infection.

39. A method according to paragraph 38, wherein the viral pulmonary infection is a SARS-COV-2 pulmonary infection.

40. A method according to any of paragraphs 35 to 39, wherein the vitamin A is administered systemically to the subject.

41. A method according to any of paragraphs 35 to 40, wherein the vitamin A is administered orally or intravenously to the subject.

42. A method according to any of paragraphs 35 to 39, wherein the vitamin A is administered to the subject by inhalation.

43. A method according to any of paragraphs 35 to 42, wherein the vitamin A is administered as a pharmaceutical composition according to any of paragraphs 26 to 28, or 30 to 32.

44. A method according to any of paragraphs 35 to 43, wherein the vitamin A comprises isolated vitamin A.

45. A method according to any of paragraphs 35 to 44 wherein the vitamin A comprises a preformed vitamin A, such as a retinyl ester or retinol.

46. A method according to any of paragraphs 35 to 45 wherein the vitamin A comprises a provitamin A, such as a carotenoid.

47. A method according to any of paragraphs 35 to 46 wherein the vitamin A comprises a bioactive form of vitamin A, such as retinal or retinoic acid.

48. A method according to any of paragraphs 35 to 47 wherein the vitamin A is administered at a dose in excess of a Tolerable Upper Limit Intake Level (UL) for the subject.

49. A method according to any of paragraphs 35 to 48 wherein the subject is a human subject.

50. A method according to paragraph 49 wherein the vitamin A is administered to the subject at a dose of >10,000 to 100,000 IU vitamin A per day.

51. A method according to paragraph 50 wherein the vitamin A is administered to the subject at a dose of about 25,000-100,000, 50,000-100,000, or 75,000-100,000 IU vitamin A per day.

52. A method according to any of paragraphs 35 to 51 wherein the vitamin A is administered to the subject once per day.

53. A method according to paragraph 52 wherein the vitamin A is administered to the subject once per day for at least 3 days from the day of first administration to the subject.

54. A method according to paragraph 53 wherein the vitamin A is administered to the subject once per day for at least a week, at least a month, or at least 6 months from the day of first administration to the subject.

55. A method according to any of paragraphs 52 to 54 wherein the vitamin A is administered to the subject for up to 6 years from the day of first administration to the subject.

56. A method according to any of paragraphs 35 to 55, wherein the subject is not vitamin A deficient.

57. A method according to any of paragraphs 35 to 56, wherein the subject has a serum retinol concentration of at least 0.7 μmol/L.

58. A method according to any of paragraphs 35 to 57, wherein the subject has a plasma concentration of vitamin A of 1-2 μmol/L.

59. A method according to any of paragraphs 35 to 58, wherein the vitamin A is administered to the subject at a dose that results in a plasma concentration of vitamin A in the subject in excess of 2 μmol/L.

60. Vitamin A for use, or use of vitamin A, according to any of paragraphs 1 to 25, wherein the vitamin A inhibits scar tissue formation by anti-inflammatory action.

61. Vitamin A for use, or use of vitamin A, according to any of paragraphs 1 to 25, or 60, wherein the vitamin A inhibits scar tissue formation by increasing expression of collagenase.

62. Vitamin A for use, or use of vitamin A, according to any of paragraphs 1 to 25, 60, or 61, wherein the scar tissue is actively maintained scar tissue.

63. Vitamin A for use, or use of vitamin A, according to any of paragraphs 1 to 25, or 60 to 62, for administration for at least five weeks from the day of first administration to the subject.

64 Vitamin A for use, or use of vitamin A, according to any of paragraphs 1 to 25, or 60 to 63, for administration to the subject at a dose upto 50% of a minimum toxic dose for the subject.

65. Vitamin A for use, or use of vitamin A, according to any of paragraphs 1 to 25, or 60 to 64 for administration to the subject at a dose of at least 5% of a minimum toxic dose for the subject.

66. A method according to any of paragraphs 35 to 59, wherein the vitamin A is administered for at least five weeks from the day of first administration to the subject.

67. A method according to any of paragraphs 35 to 59, or 66, wherein the vitamin A is administered to the subject at a dose upto 50% of a minimum toxic dose for the subject.

68. A method according to any of paragraphs 35 to 59, 66, or 67, wherein the vitamin A is administered to the subject at a dose of at least 5% of a minimum toxic dose for the subject.

69. A method according to any of paragraphs 35 to 59, or 66 to 68, wherein the vitamin A inhibits scar tissue formation by anti-inflammatory action.

70. A method according to any of paragraphs 35 to 59, or 66 to 69, wherein the vitamin A inhibits scar tissue formation by increasing expression of collagenase.

71. A method according to any of paragraphs 35 to 59, or 66 to 68, wherein the scar tissue is actively maintained scar tissue.

72. Vitamin A for use, or use of vitamin A, according to any of paragraphs 1 to 25, or 60 to 65, for administration to the subject at a maintenance dose, wherein the maintenance dose is less than a full treatment dose.

73. Vitamin A for use, or use of vitamin A, according to paragraph 72, wherein the maintenance dose is up to three quarters of a full treatment dose.

74. Vitamin A for use, or use of vitamin A, according to paragraph 72 or 73, wherein the maintenance dose is up to two-thirds of a full treatment dose.

75. Vitamin A for use, or use of vitamin A, according to any of paragraphs 72 to 74, wherein the maintenance dose is at least a quarter of a full treatment dose.

76. Vitamin A for use, or use of vitamin A, according to any of paragraphs 72 to 75, wherein the maintenance dose is to be administered after the subject has been administered one or more full-treatment doses.

77. Vitamin A for use, or use of vitamin A, according to any of paragraphs 72 to 76, wherein the maintenance dose is to be administered from the day after the last administration of a full-treatment dose to the subject.

78. Vitamin A for use, or use of vitamin A, according to any of paragraphs 72 to 77, wherein a plurality of maintenance doses is to be administered.

79. Vitamin A for use, or use of vitamin A, according to paragraph 78, wherein the maintenance doses are to be administered for at least four weeks from the day of first administration of a maintenance dose to the subject.

80. Vitamin A for use, or use of vitamin A, according to paragraph 79, wherein the maintenance doses are to be administered for at least 12 weeks from the day of first administration of a maintenance dose to the subject.

81. Vitamin A for use, or use of vitamin A, according to paragraph 79, wherein the maintenance doses are to be administered for at least 6 months from the day of first administration of a maintenance dose to the subject.

82. Vitamin A for use, or use of vitamin A, according to paragraph 79, wherein the maintenance doses are to be administered for at least 12 months from the day of first administration of a maintenance dose to the subject.

83. Vitamin A for use, or use of vitamin A, according to any of paragraphs 72 to 82, wherein the maintenance doses are to be administered for up to 6 years from the day of first administration of a maintenance dose to the subject.

84. Vitamin A for use, or use of vitamin A, according to any of paragraphs 72 to 83, wherein the subject is a human subject.

85. Vitamin A for use, or use of vitamin A, according to paragraph 84, wherein the, or each maintenance dose comprises >2,500 IU to 75,000 IU vitamin A per day.

86. Vitamin A for use, or use of vitamin A, according to paragraph 84 or 85, wherein the, or each maintenance dose comprises 5,000-75,000, 10,000-75,000, or 20,000-75,000 IU vitamin A per day.

87. Vitamin A for use, or use of vitamin A, according to paragraph 84 or 85, wherein the, or each maintenance dose comprises 5,000-50,000, 10,000-50,000, or 20,000-50,000 IU vitamin A per day.

88. A multiple-dose formulation which comprises a plurality of separate unit doses of vitamin A, wherein each unit dose is a maintenance dose of vitamin A, and wherein each unit dose comprises >2,500 IU to 75,000 IU vitamin A.

89. A multiple-dose formulation according to paragraph 88, wherein the, or each unit dose comprises 5,000-75,000, 10,000-75,000, or 20,000-75,000 IU vitamin A.

90. A multiple-dose formulation according to paragraph 88 or 89, wherein the, or each unit dose comprises 5,000-50,000, 10,000-50,000, or 20,000-50,000 IU vitamin A.

91. A multiple-dose formulation according to any of paragraphs 88 to 90 for administration to a human subject.

92. A multiple-dose formulation according to any of paragraphs 88 to 91, wherein the vitamin A comprises isolated vitamin A.

93. A multiple-dose formulation according to any of paragraphs 88 to 92, wherein the vitamin A comprises a preformed vitamin A, such as a retinyl ester or retinol.

94. A multiple-dose formulation according to any of paragraphs 88 to 93, wherein the vitamin A comprises a provitamin A, such as a carotenoid.

95. A multiple-dose formulation according to any of paragraphs 88 to 94, wherein the vitamin A comprises a bioactive form of vitamin A, such as retinal or retinoic acid.

96. A multiple-dose formulation according to any of paragraphs 88 to 95, wherein the vitamin A is part of a pharmaceutical composition comprising vitamin A and a pharmaceutically acceptable carrier, excipient or diluent.

97. A multiple-dose formulation according to paragraph 96, wherein vitamin A is the only non-cellular, non-antibiotic, active agent present in the pharmaceutical composition.

98. A multiple-dose formulation according to any of paragraphs 88 to 97 comprising at least 7, at least 30, or at least 100 separate unit doses of vitamin A.

99. A multiple-dose formulation which comprises:
a first plurality of separate unit doses of vitamin A, wherein each unit dose of the first plurality of separate unit doses is a full treatment unit dose of vitamin A, which comprises >10,000 IU to 100,000 IU vitamin A; and
a second plurality of separate unit doses of vitamin A, wherein each unit dose of the second plurality of separate unit doses is a maintenance dose of vitamin A, which comprises less vitamin A than a full treatment unit dose, and wherein each unit dose comprises >2,500 IU to 75,000 IU vitamin A.

100. A multiple-dose formulation according to paragraph 99, wherein each unit dose of the first plurality of separate unit doses is a full treatment dose of vitamin A, which comprises 25,000-100,000, 50,000-100,000, or 75,000-100,000 IU vitamin A.

101. A multiple-dose formulation according to paragraph 99 or 100, wherein each unit dose of the second plurality of separate unit doses is a maintenance dose as recited in any of paragraphs 72 to 75, or 85 to 97.

102. A multiple-dose formulation according to any of paragraphs 88 to 101 for use as a medicament.

103. A multiple-dose formulation according to any of paragraph 88 to 101 for use in the prevention, treatment, or amelioration of pulmonary fibrosis.

104. Use of a multiple-dose formulation according to any of paragraphs 88 to 101 in the manufacture of a medicament for the prevention, treatment, or amelioration of pulmonary fibrosis.

105. A multiple-dose formulation for use according to paragraph 103, or use according to paragraph 104, wherein the multiple-dose formulation inhibits formation of pulmonary scar tissue.

106. A multiple-dose formulation for use according to paragraph 103 or 105, or use according to paragraph 104 or 105, wherein the pulmonary fibrosis is associated with a pulmonary infection.

107. A multiple-dose formulation for use, or use according to any of paragraphs 103-106, wherein the pulmonary infection is a viral pulmonary infection.

108. A multiple-dose formulation for use, or use according to paragraph 107, wherein the viral pulmonary infection is a severe acute respiratory syndrome coronavirus 2 (SARS-CoV-2) pulmonary infection.

109. A multiple-dose formulation for use, or use according to any of paragraphs 103-108, wherein the vitamin A comprises isolated vitamin A.

110. A multiple-dose formulation for use, or use according to any of paragraphs 103-109, wherein the vitamin A comprises a preformed vitamin A, such as a retinyl ester or retinol.

111. A multiple-dose formulation for use, or use according to any of paragraphs 103-110, wherein the vitamin A comprises a provitamin A, such as a carotenoid.

112. A multiple-dose formulation for use, or use according to any of paragraphs 103-111, wherein the vitamin A comprises a bioactive form of vitamin A, such as retinal or retinoic acid.

113. A multiple-dose formulation for use, or use according to any of paragraphs 103-112, for administration to a human subject.

114. A method according to any of paragraphs 35-59, or 66 to 71, wherein the subject is administered a maintenance dose of vitamin A, wherein the maintenance dose is less than a full treatment dose.

115. A method according to paragraph 114, wherein the maintenance dose is up to three quarters of a full treatment dose.

116. A method according to paragraph 114 or 115, wherein the maintenance dose is up to two-thirds of a full treatment dose.

117. A method according to any of paragraphs 114 to 116, wherein the maintenance dose is at least a quarter of a full treatment dose.

118. A method according to any of paragraphs 114 to 117, wherein the maintenance dose is administered after the subject has been administered one or more full-treatment doses.

119. A method according to any of paragraphs 114 to 118, wherein the maintenance dose is administered from the day after the last administration of a full-treatment dose to the subject.

120. A method according to any of paragraphs 114 to 119, wherein a plurality of maintenance doses administered to the subject.

121. A method according to paragraph 120, wherein the maintenance doses are administered for at least four weeks from the day of first administration of a maintenance dose to the subject.

122. A method according to paragraph 121, wherein the maintenance doses are administered for at least 12 weeks from the day of first administration of a maintenance dose to the subject.

123. A method according to paragraph 121, wherein the maintenance doses are administered for at least 6 months from the day of first administration of a maintenance dose to the subject.

124. A method according to paragraph 121, wherein the maintenance doses are administered for at least 12 months from the day of first administration of a maintenance dose to the subject.

125. A method according to any of paragraphs to 120 to 124, wherein the maintenance doses are administered for up to 6 years from the day of first administration of a maintenance dose to the subject.

126. A method according to any of paragraphs to 114 to 125, wherein the subject is a human subject.

127. A method according to paragraph 126, wherein the, or each maintenance dose comprises >2,500 IU to 75,000 IU vitamin A per day.

128. A method according to paragraph 126 or 127, wherein the, or each maintenance dose comprises 5,000-75,000, 10,000-75,000, or 20,000-75,000 IU vitamin A per day. 129. A method according to paragraph 126 or 127, wherein the, or each maintenance dose comprises 5,000-50,000, 10,000-50,000, or 20,000-50,000 IU vitamin A per day.

### Treatment of Traumatic Brain Injury

A third aspect of the invention relates to the treatment of traumatic brain injury.

This third aspect of the invention relates to compounds, compositions, combined preparations, and multiple-dose formulations, for use in the treatment of acute and chronic traumatic brain injury, and of brain disorders with delayed onset following traumatic brain injury, and to methods of treatment of such injuries and disorders using the compounds, compositions, combined preparations, or multiple-dose formulations.

Traumatic brain injury (TBI) is generally divided into acute TBI and chronic TBI. Acute TBI in sports-related trauma may lead to concussion, subconcussion, haemorrhage or other structural brain damage. Concussion, also known as mild traumatic brain injury (mTBI), is typically defined as a head injury that temporarily affects brain functioning. It may be caused by impact forces, in which the head strikes or is struck by something, or impulsive forces, in which the head moves without itself being subject to blunt trauma. Forces may cause linear, rotational, or angular movement of the brain or a combination of them. The amount of rotational force is thought to be the major component in concussion and its severity. Concussion is the most common form of acute TBI in high-impact sports. Symptoms of concussion may include loss of consciousness, memory loss, headaches, difficulty with thinking, concentration or balance, nausea, blurred vision, sleep disturbances, and mood changes. Any of these symptoms may begin immediately, or appear days after the injury. It is not unusual for symptoms to last two weeks in adults and four weeks in children.

Post-concussion syndrome (PCS) is the presence of persistent neurological symptoms lasting for more than 3 months and is observed in 40-80% of individuals exposed to mild TBI. About 10-15% of individuals experience persistent symptoms after 1 year. Neuropsychological tests reveal that cognitive impairment often persists beyond the subjectively symptomatic time in boxers following mild TBI or a knockout. The seemingly mild head injury causing these subtle subjective and objective neuropsychiatric deficits is sometimes referred to as subconcussion. There is no established treatment for PCS.

Another concussion before the symptoms of a prior concussion have resolved is associated with worse outcomes. Second impact syndrome (SIS) may develop where someone who has sustained an initial head injury, most often a concussion, sustains a second head injury days or weeks after the initial injury, and before its symptoms have fully cleared. The second head injury is typically only a minor blow to the head, but within minutes, the brain swells dangerously and can herniate. The brain stem can fail within five minutes. Except in boxing, all cases have occurred in athletes under the age of 20. Due to the very small number of documented cases, however, the diagnosis is controversial.

Concussion may lead to deleterious effects including reduced brain resistance to a variety of brain disorders with delayed onset. There is a strong positive correlation between concussion and depression, Parkinson's disease, and anxiety disorders. The severity of concussions and their symptoms may worsen with successive injuries, even if a subsequent injury occurs months or years after an initial one. Repetitive TBI is firmly linked with dementia. Cumulative effects may include psychiatric disorders and loss of long-term memory. For example, the risk of developing clinical depression has been found to be significantly greater for retired American football players with a history of three or more concussions than for those with no concussion history. Three or more concussions is also associated with a fivefold greater chance of developing Alzheimer's disease earlier and a threefold greater chance of developing memory deficits.

Chronic traumatic encephalopathy (CTE) is a neurodegenerative disease caused by repeated head injuries. The condition was previously referred to as “dementia pugilistica”, or “punch drunk” syndrome, as it was first noted in boxers. Symptoms do not typically begin until years after the injuries. The disease can lead to cognitive and physical handicaps such as parkinsonism, speech and memory problems, slowed mental processing, tremor, depression, and inappropriate behaviour. It shares features with Alzheimer's disease. Most documented cases have occurred in athletes involved in contact sports such as boxing, American football, professional wrestling, ice hockey, rugby, and soccer. The exact amount of trauma required for the condition to occur is unknown, and definitive diagnosis can currently only occur at autopsy.

CTE is classified as a tauopathy. The neuropathological appearance of CTE is distinguished from other tauopathies, such as Alzheimer's disease. The macroscopic features of CTE include diffuse brain atrophy, ventricular dilatation, cavum septum pellucidum with or without fenestrations, cerebellar scarring and depigmentation and degeneration of the substantia nigra. Marked atrophy of the medial temporal lobe, thalamus, hypothalamus and mammillary bodies becomes evident in advanced CTE. CTE pathology at the microscopic level includes extensive neurofibrillary tangles (NFTs) composed of mixed 3-repeat (3R) and 4-repeat (4R) tau isoforms. NFTs and astrocytic tangles in CTE are most abundant in the frontal and temporal cortices. Although both are mixed 3R and 4R tauopathies, CTE is distinct from Alzheimer's disease in the lack of, or relatively little, AB deposition especially in younger individuals and in early stages of CTE.

Astrocytic tau pathology in CTE is predominantly 4R tau and is more widely distributed than that observed in ageing and Alzheimer's disease (see Ling et al., “Neurological consequences of traumatic brain injuries in sports”, Molecular and Cellular Neuroscience 66 (2015) 114:122). No cure currently exists for CTE. Treatment is supportive as with other forms of dementia.

Inappropriate management of concussion and subconcussion may put an athlete at risk of developing SIS and/or chronic PCS (CPCS) with persistent neurological symptoms, most commonly, headache, dizziness, impaired attention, poor memory, executive dysfunction, irritability and depression. CPCS is a type of chronic TBI, which is probably distinct from CTE, and the onset of neurological symptoms begins rapidly after the head trauma and persists but rarely progresses.

The response of neural tissue to concussion is not well characterised. It is known that mild trauma to the brain causes biochemical changes resulting in neural dysfunction and structural abnormalities. When subjected to rapid acceleration, deceleration and rotational forces, the brain and all its components, including neurons, glial cells and blood vessels, are stretched, which may disrupt their normal functions.

Mechanical shearing and stretching forces disrupt the cell membrane of nerve cells through “mechanoporation”. This results in potassium efflux into the extracellular space with the subsequent release of excitatory neurotransmitters, leading to sustained depolarization, impaired nerve activity, and potential nerve damage. In an effort to restore ion balance, sodium-potassium ion pumps increase activity, which results in excessive glucose consumption, quickly depleting glucose stores within the cells. Inefficient oxidative metabolism leads to anaerobic metabolism of glucose and increased lactate accumulation. The resultant local acidosis in the brain and increased cell membrane permeability leads to local swelling. After this increase in glucose metabolism, there is a subsequent lower metabolic state which may persist for up to four weeks after injury.

Axonal swellings occur and axons become disconnected at the location of the injury. Axons that span long distances from the cell bodies are particularly susceptible to stretching, which may lead to diffuse axonal injury. It is possible that concussion leads to axonal injury, loss of microvascular integrity and breach of the blood brain barrier, triggering an inflammatory cascade and microglia and astrocyte activation, forming the basis of a mechanistic link with the subsequent development of chronic traumatic encephalopathy (CTE) (Ling et al., “Neurological consequences of traumatic brain injuries in sports”, Molecular and Cellular Neuroscience 66 (2015) 114:122).

Tissue repair encompasses two separate processes: regeneration and replacement. Regeneration refers to a type of healing in which new growth completely restores portions of damaged tissue to their normal state. Replacement refers to a type of healing in which severely damaged or non-regenerable tissues are repaired by the laying down of connective tissue (or glial tissue in the brain), a process commonly referred to as scarring. Tissue repair may restore some of the original structures of the damaged tissue, but may also result in structural abnormalities that impair function.

In the central nervous system (CNS), glial scar grows as a major physical and chemical barrier against regeneration of neurons as it forms dense isolation and creates an inhibitory environment, resulting in limitation of optimal neural function. Glial scar is mainly attributed to the activation of resident astrocytes which surround the lesion core and wall off intact neurons. Glial cells induce the infiltration of immune cells, resulting in transient increase in extracellular matrix deposition and inflammatory factors which inhibit axonal regeneration, impede functional recovery, and may contribute to the occurrence of neurological complications.

Traumatic brain injury causes death of neurons and glia around the site of the injury, shearing of ascending and descending axons, and damage to the vasculature. This leads to haemorrhage at the lesion, and release of factors associated with glial scar formation and immune response. Astrocytes and microglia quickly begin to accumulate around the lesion and increase the expression of pro-inflammatory cytokines and chemokines that inhibit axonal regeneration. Increased levels of pro-inflammatory cytokines, myelin debris, and chondroitin sulphate proteoglycans (CSPGs) in the glial scar contribute to secondary damage to neurons, oligodendrocytes, and dystrophic endings of axonal dieback and inhibit the recovery. Perivascular fibroblasts are attracted by haematogenous macrophages, which infiltrate the lesion, and the perivascular fibroblasts form the fibrotic part of the scar (Wang et al., “Portrait of glial scar in neurological diseases”, International Journal of Immunopathology and Pharmacology, Vol. 31, 1-6, 2018).

Strategies to treat concussion are merely those to alleviate the symptoms by physical and cognitive rest. The rationale for rest is that during the acute post-injury period of increased metabolic demand and limited ATP reserves, non-essential activity draws oxygen and glycogen away from damaged neural tissue and slows regeneration and replacement. Other approaches to treatment of concussion are specific to different clinical subtypes. Paracetamol or NSAIDs may be recommended to help with a headache. For patients who experience vision impairments, specific rehabilitation interventions may be used involving exposures to various stimuli. Physiotherapy may be useful for persistent balance problems; cognitive behavioural therapy may be useful for mood changes. For chronic concussion, pharmacological intervention is normally used. For acute or sub-acute phases after injury, a “wait and see” approach is usually implemented due to the heterologous nature of concussion and hence its individual requirements for treatment.

It is clear that there is no effective treatment for acute and chronic TBI other than attempts to alleviate the obvious symptoms of these conditions. There is a need, therefore, to provide improved treatments for acute and chronic TBI.

The applicant has appreciated that inhibition of glial scar tissue formation following TBI may be a key aspect of the repair process, and in particular is a necessary prerequisite for successful tissue regeneration. The applicant has also recognised that vitamin A may be used to inhibit glial scar tissue formation, and may thus be used in the effective treatment of acute and chronic TBI, and of brain disorders with delayed onset following TBI.

According to the invention there is provided Vitamin A for use in the treatment of acute or chronic traumatic brain injury (TBI) in a subject.

According to the invention there is also provided use of vitamin A in the manufacture of a medicament for the treatment of acute or chronic TBI in a subject.

There is also provided according to the invention a method of treating acute or chronic TBI in a subject, which comprises administering to the subject an effective amount of vitamin A.

Optionally the acute or chronic TBI is concussion.

Optionally the acute or chronic TBI is post-concussion syndrome (PCS).

Optionally the chronic TBI is chronic traumatic encephalopathy (CTE).

Administration of vitamin A to a subject following a TBI may also prevent, treat, or ameliorate a brain disorder with delayed onset following the TBI.

There is also provided according to the invention vitamin A for use in the prevention, treatment, or amelioration of a brain disorder with delayed onset following a TBI in a subject.

There is also provided according to the invention use of vitamin A in the manufacture of a medicament for the prevention, treatment, or amelioration of a brain disorder with delayed onset following a TBI in a subject.

The invention also provides method of preventing, treating, or ameliorating a brain disorder with delayed onset following a TBI in a subject, which comprises administering to the subject an effective amount of vitamin A.

The TBI may be an acute or chronic TBI, such as concussion, PCS, or CTE. Optionally the acute or chronic TBI is concussion. Optionally the acute or chronic TBI is PCS.

Optionally the brain disorder with delayed onset is CTE, depression, Parkinson's disease, dementia, or an anxiety disorder.

In particular, vitamin A is able to treat acute or chronic TBI, or to prevent, treat, or ameliorate a brain disorder with delayed onset following a TBI, by inhibiting formation of glial scar tissue in the brain of the subject following the TBI.

Optionally the TBI was sustained by the subject during participation in a sport.

Optionally the subject is an athlete, or was an athlete when the TBI was sustained.

Mild TBI (concussion) is a relatively common occurrence in several sports, especially contact sports, such as boxing, American football, rugby, soccer, baseball, softball, basketball, as well as other sports, including cycling, water sports, winter sports, horse riding, hockey, ball sports, skating (see Table 2 of Ling et al (supra) for a list of top 20 sports and recreational activities with the highest risk of head injuries requiring hospital emergency care or evaluation). Optionally the TBI was sustained by the subject during participation in a contact sport.

Optionally the vitamin A inhibits scar tissue formation by anti-inflammatory action.

Optionally the vitamin A inhibits scar tissue formation by increasing expression of collagenase.

Vitamin A may cause a reduction in the amount of scar tissue already formed after an injury has occurred.

Optionally the scar tissue is actively maintained scar tissue.

Vitamin A is the name of a group of fat-soluble retinoids, including retinol, retinal, and retinyl esters. There are two different categories of vitamin A. The first category, preformed vitamin A, comprises retinol and its esterified form, retinyl ester. The second category, provitamin A, comprises provitamin A carotenoids such as alpha-carotene, beta-carotene and beta-cryptoxanthin. Both retinyl esters and provitamin A carotenoids are converted to retinol, which is oxidized to retinal and then to retinoic acid. Both provitamin A and preformed vitamin A are known be metabolized intracellularly to retinal and retinoic acid, the bioactive forms of vitamin A.

Vitamin A for use according to the invention may be an isolated form of vitamin A. An isolated form of vitamin A is any form of vitamin A found in the diet or a metabolized form thereof. For example, vitamin A may be isolated from fish liver oil. Vitamin A may comprise a preformed vitamin A such as retinol or a retinyl ester. Retinyl esters include retinyl acetate and retinyl palmitate. Vitamin A may comprise a provitamin A, such as a provitamin A carotenoid including alpha-carotene, beta-carotene or beta-cryptoxanthin. Vitamin A may comprise a bioactive form of vitamin A such as retinal or retinoic acid.

Vitamin A is available for human consumption in multivitamins and as a stand-alone supplement, often in the form of retinyl acetate or retinyl palmitate. A portion of the vitamin A in some supplements is in the form of beta-carotene and the remainder is preformed vitamin A; others contain only preformed vitamin A or only beta-carotene. Supplement labels usually indicate the percentage of each form of the vitamin. The amounts of vitamin A in stand-alone supplements range widely. Multivitamin supplements typically contain 2,500 to 10,000 international units (IU) vitamin A, often in the form of both retinol and beta-carotene.

Vitamin A is listed on food and supplement labels in international units (IUs). However, Recommended Dietary Allowance (RDA) (average daily level of intake sufficient to meet the nutrient requirements of nearly all (97%-98%) healthy individuals) for vitamin A is given as micrograms (μg; mcg) of retinol activity equivalents (RAE) to account for the different bioactivities of retinol and provitamin A carotenoids (see Table 1). Because the body converts all dietary sources of vitamin A into retinol, 1 mcg of physiologically available retinol is equivalent to the following amounts from dietary sources: 1 mcg of retinol, 12 mcg of beta-carotene, and 24 mcg of alpha-carotene or beta-cryptoxanthin. From dietary supplements, the body converts 2 mcg of beta-carotene to 1 mcg of retinol.

Conversion rates between mcg RAE and IU are as follows:
1 IU retinol=0.3 mcg RAE;
1 IU beta-carotene from dietary supplements=0.15 mcg RAE;
1 IU beta-carotene from food=0.05 mcg RAE; and
1 IU alpha-carotene or beta-cryptoxanthin=0.025 mcg RAE.

An RAE cannot be directly converted into an IU without knowing the source(s) of vitamin A. For example, the RDA of 900 mcg RAE for adolescent and adult men is equivalent to 3,000 IU if the food or supplement source is preformed vitamin A (retinol). However, this RDA is also equivalent to 6,000 IU of beta-carotene from supplements, 18,000 IU of beta-carotene from food, or 36,000 IU of alpha-carotene or beta-cryptoxanthin from food. So a mixed diet containing 900 mcg RAE provides between 3,000 and 36,000 IU of vitamin A, depending on the foods consumed.

TABLE 1
Recommended Dietary Allowances (RDAs) for Vitamin A

| Age | | Male | Female | Pregnancy | Lactation |
|---|---|---|---|---|---|
| 0-6 | months* | 400 mcg RAE | 400 mcg RAE | | |
| 7-12 | months* | 500 mcg RAE | 500 mcg RAE | | |
| 1-3 | years | 300 mcg RAE | 300 mcg RAE | | |
| 4-8 | years | 400 mcg RAE | 400 mcg RAE | | |
| 9-13 | years | 600 mcg RAE | 600 mcg RAE | | |
| 14-18 | years | 900 mcg RAE | 700 mcg RAE | 750 mcg RAE | 1,200 mcg RAE |
| 19-50 | years | 900 mcg RAE | 700 mcg RAE | 770 mcg RAE | 1,300 mcg RAE |
| 51+ | years | 900 mcg RAE | 700 mcg RAE | | |

*Adequate Intake (AI), equivalent to the mean intake of vitamin A in healthy, breastfed infants.
Source: National Institutes of Health, Vitamin A, Fact Sheet for Health Professionals, as updated 5 Oct. 2018

The Food and Nutrition Board (FNB) at the Institute of Medicine of the National Academies (formerly National Academy of Sciences) has established tolerable Upper Intake Level (UL) (maximum daily intake unlikely to cause adverse health effects) for preformed vitamin A that apply to both food and supplement intakes. The FNB based these ULs on the amounts associated with an increased risk of liver abnormalities in men and women, teratogenic effects, and a range of toxic effects in infants and children. The FNB has not established ULs for beta-carotene and other provitamin A carotenoids.

TABLE 2
Tolerable Upper Intake Levels (ULs) for Preformed Vitamin A*

| Age | | Male | Female | Pregnancy | Lactation |
|---|---|---|---|---|---|
| 0-12 | months | 600 mcg RAE | 600 mcg RAE | | |
| | | (2,000 IU) | (2,000 IU) | | |
| 1-3 | years | 600 mcg RAE | 600 mcg RAE | | |
| | | (2,000 IU) | (2,000 IU) | | |
| 4-8 | years | 900 mcg RAE | 900 mcg RAE | | |
| | | (3,000 IU) | (3,000 IU) | | |
| 9-13 | years | 1,700 mcg RAE | 1,700 mcg RAE | | |
| | | (5,667 IU) | (5,667 IU) | | |
| 14-18 | years | 2,800 mcg RAE | 2,800 mcg RAE | 2,800 mcg RAE | 2,800 mcg RAE |
| | | (9,333 IU) | (9,333 IU) | (9,333 IU) | (9,333 IU) |
| 19+ | years | 3,000 mcg RAE | 3,000 mcg RAE | 3,000 mcg RAE | 3,000 mcg RAE |
| | | (10,000 IU) | (10,000 IU) | (10,000 IU) | (10,000 IU) |

Source: National Institutes of Health, Vitamin A, Fact Sheet for Health Professionals, as updated 5 Oct. 2018
*These ULs, expressed in mcg and in IUs (where 1 mcg = 3.33 IU), only apply to products from animal sources and supplements whose vitamin A comes entirely from retinol or ester forms, such as retinyl palmitate. However, many dietary supplements (such as multivitamins) do not provide all of their vitamin A as retinol or its ester forms. For example, the vitamin A in some supplements consists partly or entirely of beta-carotene or other provitamin A carotenoids. In such cases, the percentage of retinol or retinyl ester in the supplement should be used to determine whether an individual's vitamin A intake exceeds the UL. For example, a supplement labeled as containing 10,000 IU of vitamin A with 60% from beta-carotene (and therefore 40% from retinol or retinyl ester) provides 4,000 IU of preformed vitamin A. That amount is above the UL for children from birth to 13 years but below the UL for adolescents and adults.

The Vitamin A may be provided from a mixture of different sources, including, for example, in normal feed, and in the form of a supplement.

It will be appreciated that use of a natural vitamin for treatment of acute or chronic TBI, or for prevention, treatment, or amelioration of a brain disorder with delayed onset following a TBI, is particularly advantageous because of its known safety profile.

Preferably vitamin A for use according to the invention, or use of vitamin A according to the invention, comprises a high dose of vitamin A.

A high dose of vitamin A is considered to be a dose that exceeds a UL for the subject. Examples of high doses of vitamin A include: >10,000 IU to 100,000 IU vitamin A per day; about 25,000 to 50,000 IU vitamin A per day; about 25,000 to 75,000 IU vitamin A per day; about 25,000 to 100,000 IU vitamin A per day; about 50,000 to 100,000 IU vitamin A per day; or about 75,000 to 100,000 IU vitamin A per day, in particular of preformed vitamin A.

A high dose of vitamin A may be a dose that is 5%-50% of a minimum toxic dose for the subject.

Vitamin A may be administered to the subject at least once per day.

Vitamin A may be administered to the subject once per day, twice per day, three times per day, four times per day, or five times per day.

Vitamin A may be administered to the subject for at least 3 days for example for at least a week, for at least a month, or for at least 6 months from the day of first administration to the subject.

Vitamin A may be administered to the subject for at least five weeks from the day of first administration to the subject.

Vitamin A may be administered to the subject for at least five weeks from the day of first administration to the subject for treatment of an injury that occurred over six months prior to the date of first administration of Vitamin A to the subject in accordance with the invention.

Prolonged exposure to high doses of vitamin A may lead to hypervitaminosis A. Thus, it may be preferred to limit administration of high doses of vitamin A to the subject for up to 6 years, or up to 6 months, from the day of first administration to the subject.

Optionally for an adult human subject (>18 years old), the subject may be administered up to 100,000 IU vitamin A (in particular of preformed vitamin A) per day for up to 6 months. For example, >10,000 IU to 100,000 IU vitamin A per day; about 25,000 to 100,000 IU vitamin A per day; about 50,000 to 100,000 IU vitamin A per day; or about 75,000 to 100,000 IU vitamin A per day (in particular of preformed vitamin A) for up to 6 months.

Optionally for an adult human subject (>18 years old), the subject may be administered up to 25,000 IU vitamin A per day (in particular of preformed vitamin A) for up to 6 years. For example, >10,000 IU to 25,000 IU vitamin A per day (in particular of preformed vitamin A) for up to 6 years.

Ongoing administration (for example, ongoing daily administration) of vitamin A for weeks, months, or years, to the subject may be particularly effective in preventing, or reducing the risk of, the subject developing a brain disorder with delayed onset, such as Parkinson's disease, CTE, depression, an anxiety disorder, or dementia.

Optionally the subject is administered up to 50% (for example >10% to 50%, or 25% to 50%) of a maximum safe dose of vitamin A (in particular of preformed vitamin A) for the subject per day.

For example, a maximum safe dose of vitamin A (in particular of preformed vitamin A) per day for an adult human subject may be 100,000 IU vitamin A (in particular of preformed vitamin A).

Optionally the subject may be administered a dose of vitamin A which is upto 50% of a minimum toxic dose for the subject.

Optionally the subject may be administered a dose of vitamin A which is at least 5% of a minimum toxic dose for the subject.

For example, a minimum toxic dose for a subject may be 1,000 IU/kg body weight (BW)/day.

The vitamin A may be administered to a subject systemically, for example, orally or intravenously.

Without being bound by theory, it is believed that inhibition of glial scar tissue formation in accordance with the invention facilitates regeneration of normal tissue, in particular by stem cells and/or quiescent cells present within or near damaged tissue. Quiescence is the reversible state of a cell in which it does not divide but retains the ability to re-enter cell proliferation. Some adult stem cells are maintained in a quiescent state and can be rapidly activated when stimulated, for example by damage or injury to the tissue in which they reside.

It will be appreciated that vitamin A should preferably be administered as soon as possible after a traumatic brain injury has occurred. Optionally vitamin A is administered within a month, within a week, within a day, within a few hours (for example, within 12 or 6 hours), or within an hour of the traumatic brain injury causing concussion.

However, beneficial effects of treatment with vitamin A may also be observed when treatment is initiated many weeks, months, or even years after an injury has occurred. Thus, optionally vitamin A may be administered weeks, months, years or even decades after an injury has occurred, for example within six weeks, six months, a year, or a decade, or within twenty, thirty, forty, fifty, or sixty years of the injury.

Optionally the subject is not vitamin A deficient.

Plasma retinol levels are typically measured to assess vitamin A status. However, plasma retinol levels are under tight hepatic homeostatic control and do not decline until vitamin A concentration in the liver is almost depleted (critical liver concentration≤20 μg g-1 of liver). Liver vitamin A reserves can be measured indirectly through the relative dose-response test (McLaren, D. S.; Kraemer, K. Manual on Vitamin Deficiency Disorders (VADD), 3rd ed.; Sight and Life Press:Basel, Switzerland, 2012; ISBN 978-3-906412-58-0), which is considered the “gold standard” indicator of whole-body vitamin A status. However, for clinical purposes, plasma retinol levels alone are sufficient and commonly used for documenting significant deficiency of vitamin A. The physiological plasma concentration of vitamin A is 1-2 μmol/L and, according to the World Health Organization, values of serum retinol concentrations below a cut-off of 0.70 μmol/L (or 20 μg/dL) represent biochemical vitamin A deficiency (VAD), and values lower than 0.35 μmol/L are indicative of severe deficiency and associated with numerous clinical manifestations.

Optionally the subject has a serum retinol concentration of at least 0.7 μmol/L.

Optionally the subject has a plasma concentration of vitamin A of 1-2 μmol/L.

Optionally the vitamin A is to be administered to the subject at a dose that results in a plasma concentration of vitamin A in excess of 2 μmol/L.

Also provided according to the invention is a multiple-dose formulation comprising a plurality of separate unit doses of vitamin A wherein each unit dose comprises up to 100,000 IU vitamin A, for example >10,000 IU to 100,000 IU vitamin A; 25,000 to 50,000 IU vitamin A, 25,000 to 75,000 IU vitamin A, 25,000 to 100,000 IU vitamin A; 50,000 to 100,000 IU vitamin A; or 75,000 to 100,000 IU vitamin A (in particular of preformed vitamin A).

Vitamin A of a multiple-dose formulation of the invention may comprise any combination of vitamin A described previously.

A multiple-dose formulation of the invention may comprise at least 7 unit doses, at least 30 unit doses, or at least 100 unit doses of vitamin A.

Each unit dose of vitamin A in a multiple-dose formulation of the invention may comprise a pharmaceutical composition comprising vitamin A and a pharmaceutically acceptable carrier, excipient or diluent.

Optionally the pharmaceutical composition is a sterile composition.

There is further provided according to the invention a multiple-dose formulation of the invention for use as a medicament.

There is also provided according to the invention a multiple-dose formulation of the invention for use in the treatment of acute or chronic traumatic brain injury (TBI) in a subject.

There is also provided according to the invention use of a multiple-dose formulation of the invention in the manufacture of a medicament for the treatment of acute or chronic TBI in a subject.

Optionally the acute or chronic TBI is concussion.

Optionally the acute or chronic TBI is post-concussion syndrome (PCS).

Optionally the chronic TBI is chronic traumatic encephalopathy (CTE).

There is also provided according to the invention a multiple-dose formulation of the invention for use in the prevention, treatment, or amelioration of a brain disorder with delayed onset following a TBI in a subject.

There is also provided according to the invention use of a multiple-dose formulation of the invention in the manufacture of a medicament for the prevention, treatment, or amelioration of a brain disorder with delayed onset following a TBI in a subject.

Optionally the brain disorder with delayed onset is CTE, depression, Parkinson's disease, dementia, or an anxiety disorder.

Optionally the vitamin A inhibits formation of glial scar tissue in the brain of the subject. Optionally the subject is a human subject.

Optionally the TBI was sustained when the subject was playing a sport.

Optionally the subject is an athlete, or was an athlete when the TBI was sustained.

There is also provided according to the invention a multiple-dose formulation of the invention for use in inhibition of glial scar tissue formation in a subject.

There is also provided according to the invention use of a multiple-dose formulation of the invention in the manufacture of a medicament for in inhibition of glial scar tissue formation in a subject.

### Maintenance Doses

We have found that administering a post-treatment maintenance dose of vitamin A supplement provides continued benefits, especially for treatment of soft tissue injuries, for example connective tissue injuries. As described in more detail in Example 10 below, after several weeks of treatment with “full-treatment doses” of vitamin A supplement, horses with connective tissue injuries (tendon or ligament) were orally administered vitamin A once per day with a reduced, “maintenance dose” of half the “full-treatment dose” of vitamin A supplement for 7 weeks. The results provide evidence for beneficial effects of continuing with a maintenance dose of vitamin A supplement after a period of administration of full-treatment doses.

Accordingly, after a period of treatment of a subject with a “full-treatment doses” of Vitamin A, it may be advantageous to continue treatment for a further period with “maintenance doses” of Vitamin A.

A “maintenance dose” is a dose of vitamin A, or of a composition comprising vitamin A, which is less than a “full-treatment dose”.

Optionally a maintenance dose is up to three quarters of a full-treatment dose, or up to two-thirds of a full treatment dose.

Optionally a maintenance dose is at least a quarter of a full-treatment dose.

Optionally a maintenance dose is to be administered after the subject has been administered one or more full-treatment doses.

Optionally a maintenance dose is to be administered from the day after the last administration of a full-treatment dose.

Typically, a plurality of maintenance doses is to be administered to the subject.

Optionally the maintenance doses are to be administered for at least four weeks from the day of first administration of a maintenance dose to the subject.

Optionally the maintenance doses are to be administered for at least 12 weeks from the day of first administration of a maintenance dose to the subject.

Optionally the maintenance doses are to be administered for at least 6 months from the day of first administration of a maintenance dose to the subject.

Optionally the maintenance doses are to be administered for at least 12 months from the day of first administration of a maintenance dose to the subject.

Optionally the maintenance doses are to be administered for up to 6 years from the day of first administration of a maintenance dose to the subject.

The, or each full-treatment dose optionally comprises a dose upto 50% of a minimum toxic dose for the subject.

The, or each full-treatment dose optionally comprises a dose of at least 5% of a minimum toxic dose for the subject.

For a human subject the, or each full-treatment dose optionally comprises >10,000 to 100,000 IU vitamin A per day.

For a human subject the, or each full-treatment dose optionally comprises about 25,000-100,000, 50,000-100,000, or 75,000-100,000 IU vitamin A per day

For a human subject the, or each maintenance dose optionally comprises >2,500 IU to 75,000 IU vitamin A per day.

For a human subject optionally the, or each maintenance dose comprises 5,000-75,000, 10,000-75,000, or 20,000-75,000 IU vitamin A per day.

For a human subject optionally the, or each maintenance dose comprises 5,000-50,000, 10,000-50,000, or 20,000-50,000 IU vitamin A per day.

A “maintenance dose” may be administered as a single dose, or in multiple dose units. For example, a maintenance dose of 6,000 IU vitamin A per day for a human may be provided as two doses of 3,000 IU vitamin A, one dose to be given in the morning, and another dose to be given in the evening.

Similarly, a “full-treatment dose” may be administered as a single dose, or in multiple dose units. For example, a full-treatment dose of 12,000 IU vitamin A per day for a human may be provided as two doses of 6,000 IU vitamin A, one dose to be given in the morning, and another dose to be given in the evening.

There is also provided according to the invention a multiple-dose formulation which comprises a plurality of separate unit doses of vitamin A, wherein each unit dose is a maintenance dose of vitamin A, and wherein each unit dose comprises >2,500 IU to 75,000 IU vitamin A.

Optionally the, or each unit dose comprises 5,000-75,000, 10,000-75,000, or 20,000-75,000 IU vitamin A.

Optionally the, or each unit dose comprises 5,000-50,000, 10,000-50,000, or 20,000-50,000 IU vitamin A.

Optionally the, or each unit dose of a multiple-dose formulation of the invention is for administration to a human subject.

Optionally the vitamin A comprises isolated vitamin A.

Optionally the vitamin A comprises a preformed vitamin A, such as a retinyl ester or retinol.

Optionally the vitamin A comprises a provitamin A, such as a carotenoid.

Optionally the vitamin A comprises a bioactive form of vitamin A, such as retinal or retinoic acid.

Optionally the vitamin A is part of a pharmaceutical composition comprising vitamin A and a pharmaceutically acceptable carrier, excipient or diluent.

Optionally the composition is a sterile composition.

Optionally the vitamin A is the only non-cellular, non-antibiotic, active agent present in the pharmaceutical composition.

Optionally a multiple-dose formulation of the invention comprises at least 7, at least 30, or at least 100 separate unit doses of vitamin A.

There is also provided according to the invention a multiple-dose formulation which comprises: a first plurality of separate unit doses of vitamin A, wherein each unit dose of the first plurality of separate unit doses is a full-treatment unit dose of vitamin A; and a second plurality of separate unit doses of vitamin A, wherein each unit dose of the second plurality of separate unit doses is a maintenance dose of vitamin A.

A multiple-dose formulation of the invention comprising a first plurality of separate unit doses and a second plurality of unit doses may be for treatment of a human subject.

For a human subject optionally each maintenance unit dose optionally comprises >2,500 IU to 75,000 IU vitamin A.

For a human subject optionally each maintenance unit dose comprises 5,000-75,000, 10,000-75,000, or 20,000-75,000 IU vitamin A.

For a human subject optionally each maintenance unit dose comprises 5,000-50,000, 10,000-50,000, or 20,000-50,000 IU vitamin A.

The term “unit dose” as used herein refers to physically discrete units suited as unitary doses for the subject to be treated. That is, the vitamin A (or composition comprising vitamin A) is formulated into discrete dose units each containing a predetermined “unit dose” quantity of vitamin A calculated to produce the desired therapeutic effect, typically in association with a required pharmaceutical carrier, excipient or diluent. It should be noted that, in some cases, two or more individual dose units in combination provide a therapeutically effective amount of the active ingredient, for example, two tablets or capsules taken together (or sequentially) may provide a therapeutically effective dose, such that the unit dose in each tablet or capsule is approximately 50% of the therapeutically effective amount.

Each unit dose of a multiple-dose formulation of the invention is typically provided as a sterile unit dose.

A multiple-dose formulation of the invention may be provided packaged in a container. The container can be, for example, a bottle (e.g., with a closure device, such as a cap), a blister pack (e.g., which can provide for enclosure of one or more doses per blister), a vial, flexible packaging (e.g., sealed Mylar or plastic bags), an ampule (for single doses in solution), a dropper, a syringe, thin film, a tube and the like. In some embodiments, a container, such as a sterile container, comprises a subject pharmaceutical composition. In some embodiments the container is a bottle or a syringe. In some embodiments the container is a bottle. In some embodiments the container is a syringe.

For example, each unit dose of the multiple-dose formulation may be provided in a separate well or blister of the container, with a foil seal covering each well/blister. In addition to the container containing the unit doses of the multiple-dose formulation, an information package insert may be included describing the use and attendant benefits of the active ingredient (for example, vitamin A or a composition comprising vitamin A) in treating the condition of interest (for example, tissue damage). Preferred compounds, compositions, and unit doses are those described herein.

There is also provided according to the invention a multiple-dose formulation of the invention for use as a medicament.

There is also provided according to the invention a multiple-dose formulation of the invention for use in the treatment of acute or chronic traumatic brain injury (TBI) in a subject.

There is also provided according to the invention use of a multiple-dose formulation of the invention in the manufacture of a medicament for the treatment of acute or chronic TBI in a subject.

Optionally the acute or chronic TBI is concussion.

Optionally the acute or chronic TBI is post-concussion syndrome (PCS).

Optionally the acute or chronic TBI is chronic traumatic encephalopathy (CTE).

There is also provided according to the invention a multiple-dose formulation of the invention for use in the prevention, treatment, or amelioration of a brain disorder with delayed onset following a TBI in a subject.

There is also provided according to the invention use of a multiple-dose formulation of the invention in the manufacture of a medicament for the prevention, treatment, or amelioration of a brain disorder with delayed onset following a TBI in a subject.

Optionally the brain disorder with delayed onset is CTE, depression, Parkinson's disease, dementia, or an anxiety disorder.

Optionally a multiple-dose formulation of the invention inhibits formation of glial scar tissue in the brain of the subject.

Optionally the subject is a human subject.

Optionally the TBI was sustained when the subject was playing a sport.

Optionally the subject is an athlete, or was an athlete when the TBI was sustained.

There is also provided according to the invention a multiple-dose formulation of the invention for use in inhibition of glial scar tissue formation in a subject.

There is also provided according to the invention use of a multiple-dose formulation of the invention in the manufacture of a medicament for in inhibition of glial scar tissue formation in a subject.

There is also provided according to the invention a method of treating acute or chronic TBI in a subject, which comprises administering to the subject an effective amount of vitamin A, wherein the subject is administered a maintenance dose of vitamin A, wherein the maintenance dose is less than a full treatment dose.

There is also provided according to the invention a method of preventing, treating, or ameliorating a brain disorder with delayed onset following a TBI in a subject, which comprises administering to the subject an effective amount of vitamin A, wherein the subject is administered a maintenance dose of vitamin A, wherein the maintenance dose is less than a full treatment dose.

Optionally the maintenance dose is up to three quarters of a full treatment dose.

Optionally the maintenance dose is up to two-thirds of a full treatment dose.

Optionally the maintenance dose is at least a quarter of a full treatment dose.

Optionally the maintenance dose is administered after the subject has been administered one or more full-treatment doses.

Optionally the maintenance dose is administered from the day after the last administration of a full-treatment dose to the subject.

Optionally a plurality of maintenance doses is administered to the subject.

Optionally the maintenance doses are administered for at least four weeks from the day of first administration of a maintenance dose to the subject.

Optionally the maintenance doses are administered for at least 12 weeks from the day of first administration of a maintenance dose to the subject.

Optionally the maintenance doses are administered for at least 6 months from the day of first administration of a maintenance dose to the subject.

Optionally the maintenance doses are administered for at least 12 months from the day of first administration of a maintenance dose to the subject.

Optionally the maintenance doses are administered for up to 6 years from the day of first administration of a maintenance dose to the subject.

Optionally the subject is a human subject.

Optionally the, or each maintenance dose comprises >2,500 IU to 75,000 IU vitamin A per day.

Optionally the, or each maintenance dose comprises 5,000-75,000, 10,000-75,000, or 20,000-75,000 IU vitamin A per day.

Optionally the, or each maintenance dose comprises 5,000-50,000, 10,000-50,000, or 20,000-50,000 IU vitamin A per day.

Use of vitamin A in accordance with the invention may be particularly effective for the treatment of older subjects. For example, a human subject may be at least 18 years old, at least 25 years old, at least 30 years old, at least 40 years old, or at least 50 years old.

Diagnosis of concussion may be based on physical and neurological examination findings, duration of unconsciousness (usually less than 30 minutes) and post-traumatic amnesia (PTA; usually less than 24 hours), and the Glasgow Coma Scale (MTBI sufferers have scores of 13 to 15) (Borg et al., “Diagnostic procedures in mild traumatic brain injury: results of the WHO Collaborating Centre Task Force on Mild Traumatic Brain Injury”, Journal of Rehabilitation Medicine, 36 (43 Suppl): 61-75, 2004). Neuropsychological tests exist to measure cognitive function (Moser, et al., “Neuropsychological evaluation in the diagnosis and management of sports-related concussion”, Archives of Clinical Neuropsychology, 22 (8): 909-16, 2007). Such tests may be administered hours, days, or weeks after the injury, or at different times to demonstrate any trend. Increasingly, athletes are also being tested pre-season to provide a baseline for comparison in the event of an injury, though this may not reduce risk or affect return to play.

A diagnosis of PCS may be made when symptoms resulting from concussion last for more than three months after the injury. The International Statistical Classification of Diseases and Related Health Problems (ICD-10) sets out criteria for post-concussion syndrome (PCS) (Boake et al. (2005). “Diagnostic criteria for postconcussional syndrome after mild to moderate traumatic brain injury”. Journal of Neuropsychiatry and Clinical Neurosciences. 17 (3): 350-6.). To meet the ICD-10 criteria, a patient has had a head injury “usually sufficiently severe to result in loss of consciousness” and then develops at least three of the following eight symptoms within four weeks: headache, dizziness, fatigue, irritability, sleep problems, concentration problems, memory problems, problems tolerating stress/emotion/alcohol. Neuropsychological tests exist to measure deficits in cognitive functioning that can result from PCS (Hall et al. (2005). “Definition, diagnosis, and forensic implications of postconcussional syndrome”. Psychosomatics. 46 (3): 195-202). The Stroop Color Test and the 2&7 Processing Speed Test (which both detect deficits in speed of mental processing) can predict the development of cognitive problems from PCS. A test called the Rivermead Postconcussion Symptoms Questionnaire, a set of questions that measure the severity of 16 different post-concussion symptoms, can be self-administered or administered by an interviewer (Mittenberg and Strauman (2000). “Diagnosis of mild head injury and the postconcussion syndrome”. Journal of Head Trauma Rehabilitation. 15 (2): 783-791). Other tests that can predict the development of PCS include the Hopkins Verbal Learning A test (HVLA) and the Digit Span Forward examination.

Corsellis et al. (“The aftermath of boxing” (1973) Psychol. Med. 3, 270-303) proposed four major criteria for diagnosis of CTE: 1. Abnormalities of the septum pellucidum (i.e., cavum, fenestrations), 2. Cerebellar scarring on the inferior surface of the lateral lobes (especially the tonsillar regions), 3. Degeneration of the substantia nigra (pallor) and 4. Widespread NFTs containing hyperphosphorylated tau in the cerebral cortex and brainstem. Two recent neuropathological criteria have since been proposed (McKee et al., 2013. “The spectrum of disease in chronic traumatic encephalopathy”. Brain 136, 43-64; Omalu et al., 2011. “Emerging histomorphologic phenotypes of chronic traumatic encephalopathy in American athletes”. Neurosurgery 69, 173-183). Omalu et al. identified four phenotypes of CTE and McKee et al. classified CTE into four pathological stages.

The effects of head trauma may be seen with the use of structural imaging. Imaging techniques include the use of magnetic resonance imaging, nuclear magnetic resonance spectroscopy, CT scan, single-photon emission computed tomography, Diffusion MRI, and Positron Emission Tomography (PET). A PET scan may also be used to evaluate tau deposition.

The term “treatment” is used herein to include a prevention or lessening of any of the symptoms of an acute or chronic TBI, such as concussion, PCS, or CTE.

Symptoms of concussion include loss of consciousness, memory loss, headaches, difficulty with thinking, concentration or balance, nausea, blurred vision, sleep disturbances, and mood changes.

Symptoms of PCS include persistent neurological symptoms, most commonly, headache, dizziness, impaired attention, poor memory, executive dysfunction, irritability depression, noise sensitivity, and anxiety.

Symptoms of CTE include unsteadiness of gait, mental confusion, slowing of muscular movements, and, occasionally, hesitancy in speech, tremors of the hands and nodding of the head. Behavioural disturbances are usually the earliest findings in CTE and may include depression, mood swings, apathy, impulsivity, aggression and suicidality. Cognitive deficits include attention and concentration impairment, memory problems, executive dysfunction and eventually dementia. Common motor symptoms are parkinsonism, tremor, dysarthria, coordination difficulties and ataxia, reflect extrapyramidal and pyramidal system and cerebellum involvements. Headache is another prominent feature but may represent comorbid CPCS (Ling et al., 2015, supra). In 2014, a large cohort of pathologically confirmed CTE delineated CTE into two clinical phenotypic presentations: one with predominant mood and behavioural symptoms in younger individuals in the third decade and another with cognitive impairment presenting in the fifth decade (Stern et al., 2013. “Clinical presentation of chronic traumatic encephalopathy”. Neurology 81, 1122-1129).

Symptoms of acute or chronic TBI, such as concussion or PCS, also include reduced brain resistance to a variety of brain disorders with delayed onset, such as CTE, depression, Parkinson's disease, dementia, and anxiety disorders. Repeated concussions may also increase the risk in later life of chronic traumatic encephalopathy (CTE), Parkinson's disease, dementia, anxiety disorders, and depression.

The vitamin A can be incorporated into a variety of formulations for therapeutic administration, more particularly by combination with appropriate, pharmaceutically acceptable carriers, pharmaceutically acceptable diluents, or other pharmaceutically acceptable excipients, and can be formulated into preparations in solid, semi solid, liquid or gaseous forms, such as tablets, capsules, powders, granules, ointments, solutions, suppositories, injections, inhalants and aerosols as appropriate.

Optionally the vitamin A is in solid form.

Optionally the vitamin A is not in an organic solution.

Optionally the vitamin A is not encapsulated by, or attached to a microparticle.

Optionally the vitamin A is not encapsulated by, or attached to a nanoparticle.

Vitamin A can be administered in the form of a pharmaceutically acceptable salt. It can also be used alone or in appropriate association, as well as in combination, with other pharmaceutically active compounds. Optionally vitamin A is administered with an antibiotic agent. Optionally vitamin A is the only non-cellular, non-antibiotic, active agent administered.

The following methods and excipients are merely exemplary and are in no way limiting.

For oral preparations, vitamin A can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Vitamin A can be formulated into preparations for injection by dissolving, suspending or emulsifying in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, a pharmaceutical composition of the present disclosure can comprise further agents such as dopamine or psychopharmacologic drugs, depending on the intended use of the pharmaceutical composition.

Pharmaceutical compositions are prepared by mixing Vitamin A having the desired degree of purity, with optional physiologically acceptable carriers, other excipients, stabilizers, surfactants, buffers and/or tonicity agents. Acceptable carriers, other excipients and/or stabilizers are nontoxic to recipients at the dosages and concentrations employed, and may include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

An aqueous formulation can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the formulation to reduce aggregation and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant can range from about 0.001% to about 1% w/V.

A lyoprotectant can also be added in order to protect a labile active ingredient against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Unit dosage (or unit dose) forms for oral administration such as syrups, elixirs, and suspensions can be provided wherein each dosage unit, for example, teaspoonful, tablespoonful, or tablet contains a predetermined amount of the active agent (i.e. vitamin A). Similarly, unit dosage forms for injection or intravenous administration can comprise vitamin A in a composition as a solution in sterile water, normal saline or another pharmaceutically acceptable carrier.

The term “unit dosage form,” (or “unit dose”), as used herein, refers to physically discrete units suitable as unitary dosages for human and animal subjects, each unit containing a predetermined quantity of vitamin A, calculated in an amount sufficient to produce the desired effect in association with a pharmaceutically acceptable diluent, carrier or vehicle.

Vitamin A can be administered as an injectable formulation. Typically, injectable compositions are prepared as liquid solutions or suspensions; solid forms suitable for solution in, or suspension in, liquid vehicles prior to injection can also be prepared.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985. The composition or formulation to be administered will, in any event, contain a quantity of vitamin A adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Ranges may be expressed herein as from “about” one particular value, and/or to another particular value. When such a range is expressed, another embodiment includes from the one particular value and/or to the other particular value. Similarly, when values are expressed as approximations, by the use of the antecedent “about”, it will be understood that the particular value forms another embodiment.

Wherever the term “vitamin A” is used herein this includes reference to “vitamin A or a pharmaceutically acceptable salt thereof”.

Methods for visualising glial scar tissue formation are known to those of ordinary skill in the art. Examples include magnetic resonance imaging (MRI). Suitable examples are described in the following documents:

### MRI in CNS Injury:

Ellingson et al., “Imaging Techniques in Spinal Cord Injury”, World Neurosurg. 2014 December; 82(6): 1351-1358;
Hu et al., “Glial scar and neuroregeneration: histological, functional, and magnetic resonance imaging analysis in chronic spinal cord injury”, Journal of Neurosurgery, 2010, 13(2) (doi.org/10.3171/2010.3.SPINE09190);
Byrnes et al., “Neuropathological Differences Between Rats and Mice after Spinal Cord Injury”, J Magn Reson Imaging. 2010 October; 32(4): 836-846.

Elements of the third aspect of the invention are defined by the following numbered paragraphs:

1. Vitamin A for use in the treatment of acute or chronic traumatic brain injury (TBI) in a subject.

2. Use of vitamin A in the manufacture of a medicament for the treatment of acute or chronic TBI in a subject.

3. Vitamin A for use according to paragraph 1, or use of vitamin A according to paragraph 2, wherein the acute or chronic TBI is concussion.

4. Vitamin A for use according to paragraph 1, or use of vitamin A according to paragraph 2, wherein the acute or chronic TBI is post-concussion syndrome (PCS).

5. Vitamin A for use according to paragraph 1, or use of vitamin A according to paragraph 2, wherein the chronic TBI is chronic traumatic encephalopathy (CTE).

6. Vitamin A for use in the prevention, treatment, or amelioration of a brain disorder with delayed onset following a TBI in a subject.

7. Use of vitamin A in the manufacture of a medicament for the prevention, treatment, or amelioration of a brain disorder with delayed onset following a TBI in a subject.

8. Vitamin A for use according to paragraph 6, or use of vitamin A according to paragraph 7, wherein the brain disorder with delayed onset is CTE, depression, Parkinson's disease, dementia, or an anxiety disorder.

9. Vitamin A for use, or use according to any preceding paragraph, wherein the vitamin A inhibits formation of glial scar tissue in the brain of the subject.

10. Vitamin A for use, or use of vitamin A, according to any preceding paragraph wherein the subject is a human subject.

11. Vitamin A for use, or use according to any preceding paragraph, wherein the TBI was sustained when the subject was playing a sport.

12. Vitamin A for use, or use according to any preceding paragraph, wherein the subject is an athlete, or was an athlete when the TBI was sustained.

13. Vitamin A for use, or use of vitamin A, according to any preceding paragraph, wherein the vitamin A comprises isolated vitamin A.

14. Vitamin A for use, or use of vitamin A, according to any preceding paragraph, wherein the vitamin A comprises a preformed vitamin A, such as a retinyl ester or retinol.

15. Vitamin A for use, or use of vitamin A, according to any preceding paragraph wherein the vitamin A comprises a provitamin A, such as a carotenoid.

16. Vitamin A for use, or use of vitamin A, according to any preceding paragraph wherein the vitamin A comprises a bioactive form of vitamin A, such as retinal or retinoic acid.

17. Vitamin A for use, or use of vitamin A, according to any preceding paragraph for administration at a dose in excess of a Tolerable Upper Limit Intake Level (UL) for the subject.

18. Vitamin A for use, or use of vitamin A, according to paragraph 17 for administration to the subject at a dose of >10,000 to 100,000 IU vitamin A per day.

19. Vitamin A for use, or use of vitamin A, according to paragraph 18 for administration to the subject at a dose of about 25,000-50,000, 25,000-75,000, 25,000-100,000, 50,000-100,000, or 75,000-100,000 IU vitamin A per day.

20. Vitamin A for use, or use of vitamin A, according to any preceding paragraph for administration to the subject once per day.

21. Vitamin A for use, or use of vitamin A, according to paragraph 20 for administration for at least 3 days from the day of first administration to the subject.

22. Vitamin A for use, or use of vitamin A, according to paragraph 20 for administration for at least a week, at least a month, or at least 6 months from the day of first administration to the subject.

23. Vitamin A for use, or use of vitamin A, according to any of paragraphs 20 to 22 for administration to the subject for up to 6 years from the day of first administration to the subject.

24. Vitamin A for use, or use of vitamin A, according to any preceding paragraph for administration systemically.

25. Vitamin A for use, or use of vitamin A, according to paragraph 24 for administration to the subject orally or intravenously.

26. A method of treating acute or chronic TBI in a subject, which comprises administering to the subject an effective amount of vitamin A.

27. A method according to paragraph 26, wherein the acute or chronic TBI is concussion.

28. A method according to paragraph 26, wherein the acute or chronic TBI is post-concussion syndrome (PCS).

29. A method according to paragraph 26, wherein the chronic TBI is chronic traumatic encephalopathy (CTE).

30. A method of preventing, treating, or ameliorating a brain disorder with delayed onset following a TBI in a subject, which comprises administering to the subject an effective amount of vitamin A.

31. A method according to paragraph 30, wherein the brain disorder with delayed onset is CTE, depression, Parkinson's disease, dementia, or an anxiety disorder.

32. A method according to any of paragraphs 26 to 31, wherein the vitamin A inhibits formation of glial scar tissue in the brain of the subject.

33. A method according to any of paragraphs 26 to 32, wherein the subject is a human subject.

34. A method according to any of paragraphs 26 to 33, wherein the TBI was sustained when the subject was playing a sport.

35. A method according to any of paragraphs 26 to 34, wherein the subject is an athlete, or was an athlete when the TBI was sustained.

36. A method according to any of paragraphs 26 to 35, wherein the vitamin A comprises isolated vitamin A.

37. A method according to any of paragraphs 26 to 36, wherein the vitamin A comprises a preformed vitamin A, such as a retinyl ester or retinol.

38. A method according to any of paragraphs 26 to 37, wherein the vitamin A comprises a provitamin A, such as a carotenoid.

39. A method according to any of paragraphs 26 to 38, wherein the vitamin A comprises a bioactive form of vitamin A, such as retinal or retinoic acid.

40. A method according to any of paragraphs 26 to 39, wherein the vitamin A is administered at a dose in excess of a Tolerable Upper Limit Intake Level (UL) for the subject.

41. A method according to paragraph 40, wherein the vitamin A is administered to the subject at a dose of >10,000 to 100,000 IU vitamin A per day.

42. A method according to paragraph 41, wherein the vitamin A is administered to the subject at a dose of about 25,000-50,000, 25,000-75,000, 25,000-100,000, 50,000-100,000, or 75,000-100,000 IU vitamin A per day.

43. A method according to any of paragraphs 26 to 42, wherein the vitamin A is administered to the subject once per day.

44. A method according to paragraph 43, wherein the vitamin A is administered to the subject once per day for at least 3 days from the day of first administration to the subject.

45. A method according to paragraph 43, wherein the vitamin A is administered to the subject once per day for at least a week, at least a month, or at least 6 months from the day of first administration to the subject.

46. A method according to any of paragraphs 43 to 45, wherein the vitamin A is administered to the subject for up to 6 years from the day of first administration to the subject.

47. A method according to any of paragraphs 26 to 46, wherein the vitamin A is administered systemically to the subject.

48. A method according to paragraph 47, wherein the vitamin A is administered orally or intravenously to the subject.

49. A method according to any of paragraphs 26 to 48, wherein the vitamin A is first administered to the subject within a week of the TBI.

50. A method according to any of paragraphs 26 to 49, wherein the vitamin A is first administered to the subject within a day of the TBI.

51. Vitamin A for use, or use of vitamin A, according to any of paragraphs 1 to 25, wherein the vitamin A inhibits scar tissue formation by anti-inflammatory action.

52. Vitamin A for use, or use of vitamin A, according to any of paragraphs 1 to 25, or 51, wherein the vitamin A inhibits scar tissue formation by increasing expression of collagenase.

53. Vitamin A for use, or use of vitamin A, according to any of paragraphs 1 to 25, 51, or 52, wherein the scar tissue is actively maintained scar tissue.

54. Vitamin A for use, or use of vitamin A, according to any of paragraphs 1 to 25, or 51 to 53, for administration for at least five weeks from the day of first administration to the subject.

55. Vitamin A for use, or use of vitamin A, according to any of paragraphs 1 to 25, or 51 to 54, for administration to the subject at a dose upto 50% of a minimum toxic dose for the subject.

56. Vitamin A for use, or use of vitamin A, according to any of paragraphs 1 to 25, or 51 to 55 for administration to the subject at a dose of at least 5% of a minimum toxic dose for the subject.

57. A method according to any of paragraphs 26 to 50, wherein the vitamin A is administered for at least five weeks from the day of first administration to the subject.

58. A method according to any of paragraphs 26 to 50, or 57, wherein the vitamin A is administered to the subject at a dose upto 50% of a minimum toxic dose for the subject.

59. A method according to any of paragraphs 26 to 50, 57, or 58, wherein the vitamin A is administered to the subject at a dose of at least 5% of a minimum toxic dose for the subject.

60. A method according to any of paragraphs 26 to 50, or 57 to 59, wherein the vitamin A inhibits scar tissue formation by anti-inflammatory action.

61. A method according to any of paragraphs 26 to 50, or 57 to 60, wherein the vitamin A inhibits scar tissue formation by increasing expression of collagenase.

62. A method according to any of paragraphs 26 to 50, or 57 to 61, wherein the scar tissue is actively maintained scar tissue.

63. Vitamin A for use, or use of vitamin A, according to any of paragraphs 1 to 25, or 51 to 56, for administration to the subject at a maintenance dose, wherein the maintenance dose is less than a full treatment dose.

64. Vitamin A for use, or use of vitamin A, according to paragraph 63, wherein the maintenance dose is up to three quarters of a full treatment dose.

65. Vitamin A for use, or use of vitamin A, according to paragraph 63 or 64, wherein the maintenance dose is up to two-thirds of a full treatment dose.

66. Vitamin A for use, or use of vitamin A, according to any of paragraphs 63 to 65, wherein the maintenance dose is at least a quarter of a full treatment dose.

67. Vitamin A for use, or use of vitamin A, according to any of paragraphs 63 to 66, wherein the maintenance dose is to be administered after the subject has been administered one or more full-treatment doses.

68. Vitamin A for use, or use of vitamin A, according to any of paragraphs 63 to 67, wherein the maintenance dose is to be administered from the day after the last administration of a full-treatment dose to the subject.

69. Vitamin A for use, or use of vitamin A, according to any of paragraphs 63 to 68, wherein a plurality of maintenance doses is to be administered.

70. Vitamin A for use, or use of vitamin A, according to paragraph 69, wherein the maintenance doses are to be administered for at least four weeks from the day of first administration of a maintenance dose to the subject.

71. Vitamin A for use, or use of vitamin A, according to paragraph 70, wherein the maintenance doses are to be administered for at least 12 weeks from the day of first administration of a maintenance dose to the subject.

72. Vitamin A for use, or use of vitamin A, according to paragraph 70, wherein the maintenance doses are to be administered for at least 6 months from the day of first administration of a maintenance dose to the subject.

73. Vitamin A for use, or use of vitamin A, according to paragraph 70, wherein the maintenance doses are to be administered for at least 12 months from the day of first administration of a maintenance dose to the subject.

74. Vitamin A for use, or use of vitamin A, according to any of paragraphs 69 to 73, wherein the maintenance doses are to be administered for up to 6 years from the day of first administration of a maintenance dose to the subject.

75. Vitamin A for use, or use of vitamin A, according to any of paragraphs 63 to 74, wherein the subject is a human subject.

76. Vitamin A for use, or use of vitamin A, according to paragraph 75, wherein the, or each maintenance dose comprises >2,500 IU to 75,000 IU vitamin A per day.

77. Vitamin A for use, or use of vitamin A, according to paragraph 75 or 76, wherein the, or each maintenance dose comprises 5,000-75,000, 10,000-75,000, or 20,000-75,000 IU vitamin A per day.

78. Vitamin A for use, or use of vitamin A, according to paragraph 75 or 76, wherein the, or each maintenance dose comprises 5,000-50,000, 10,000-50,000, or 20,000-50,000 IU vitamin A per day.

79. A multiple-dose formulation comprising a plurality of separate unit doses of vitamin A wherein each unit dose comprises up to 100,000 IU vitamin A, for example >10,000 IU to 100,000 IU vitamin A; 25,000 to 50,000 IU vitamin A, 25,000 to 75,000 IU vitamin A, 25,000 to 100,000 IU vitamin A; 50,000 to 100,000 IU vitamin A; or 75,000 to 100,000 IU vitamin A (in particular of preformed vitamin A).

80. A multiple-dose formulation according to paragraph 79, which comprises at least 7 unit doses, at least 30 unit doses, or at least 100 unit doses of vitamin A.

81. A multiple-dose formulation which comprises a plurality of separate unit doses of vitamin A, wherein each unit dose is a maintenance dose of vitamin A, and wherein each unit dose comprises >2,500 IU to 75,000 IU vitamin A (in particular of preformed vitamin A).

82. A multiple-dose formulation according to paragraph 81, wherein the, or each unit dose comprises 5,000-75,000, 10,000-75,000, or 20,000-75,000 IU vitamin A.

83. A multiple-dose formulation according to paragraph 81 or 82, wherein the, or each unit dose comprises 5,000-50,000, 10,000-50,000, or 20,000-50,000 IU vitamin A.

84. A multiple-dose formulation according to any of paragraphs 81 to 83 for administration to a human subject.

85. A multiple-dose formulation according to any of paragraphs 79 to 84, wherein the vitamin A comprises isolated vitamin A.

86. A multiple-dose formulation according to any of paragraphs 79 to 85, wherein the vitamin A comprises a preformed vitamin A, such as a retinyl ester or retinol.

87. A multiple-dose formulation according to any of paragraphs 79 to 86, wherein the vitamin A comprises a provitamin A, such as a carotenoid.

88. A multiple-dose formulation according to any of paragraphs 79 to 87, wherein the vitamin A comprises a bioactive form of vitamin A, such as retinal or retinoic acid.

89. A multiple-dose formulation according to any of paragraphs 79 to 88, wherein the vitamin A is part of a pharmaceutical composition comprising vitamin A and a pharmaceutically acceptable carrier, excipient or diluent.

90. A multiple-dose formulation according to paragraph 89, wherein vitamin A is the only non-cellular, non-antibiotic, active agent present in the pharmaceutical composition.

91. A multiple-dose formulation which comprises: a first plurality of separate unit doses of vitamin A, wherein each unit dose of the first plurality of separate unit doses is a full treatment unit dose of vitamin A as recited in paragraph 79; and a second plurality of separate unit doses of vitamin A, wherein each unit dose of the second plurality of separate unit doses is a maintenance dose of vitamin A as recited in any of paragraphs 81 to 83.

92. A multiple-dose formulation according to any of paragraphs 79 to 91 for use as a medicament.

93. A multiple-dose formulation according to any of paragraphs 79 to 91 for use in the treatment of acute or chronic traumatic brain injury (TBI) in a subject.

94. Use of a multiple-dose formulation according to any of paragraphs 79 to 91 in the manufacture of a medicament for the treatment of acute or chronic TBI in a subject.

95. A multiple-dose formulation for use according to paragraph 93, or use of a multiple-dose formulation according to paragraph 94, wherein the acute or chronic TBI is concussion.

96. A multiple-dose formulation for use according to paragraph 93, or use of a multiple-dose formulation according to paragraph 94, wherein the acute or chronic TBI is post-concussion syndrome (PCS).

97. A multiple-dose formulation for use according to paragraph 93, or use of a multiple-dose formulation according to paragraph 94, wherein the chronic TBI is chronic traumatic encephalopathy (CTE).

98. A multiple-dose formulation according to any of paragraphs 79 to 91 for use in the prevention, treatment, or amelioration of a brain disorder with delayed onset following a TBI in a subject.

99. Use of a multiple-dose formulation according to any of paragraphs 79 to 91 in the manufacture of a medicament for the prevention, treatment, or amelioration of a brain disorder with delayed onset following a TBI in a subject.

100. A multiple-dose formulation for use according to paragraph 98, or use of a multiple-dose formulation according to paragraph 99, wherein the brain disorder with delayed onset is CTE, depression, Parkinson's disease, dementia, or an anxiety disorder.

101. A multiple-dose formulation for use, or use of a multiple-dose formulation, according to any of paragraphs 93 to 100, wherein the multiple-dose formulation inhibits formation of glial scar tissue in the brain of the subject.

102. A method according to any of paragraphs 26 to 50, or 57 to 62, wherein the subject is administered a maintenance dose of vitamin A, wherein the maintenance dose is less than a full treatment dose.

103. A method according to paragraph 102, wherein the maintenance dose is up to three quarters of a full treatment dose.

104. A method according to paragraph 102 or 103, wherein the maintenance dose is up to two-thirds of a full treatment dose.

105. A method according to any of paragraphs 102 to 104, wherein the maintenance dose is at least a quarter of a full treatment dose.

106. A method according to any of paragraphs 102 to 105, wherein the maintenance dose is administered after the subject has been administered one or more full-treatment doses.

107. A method according to any of paragraphs 102 to 106, wherein the maintenance dose is administered from the day after the last administration of a full-treatment dose to the subject.

108. A method according to any of paragraphs 102 to 107, wherein a plurality of maintenance doses is administered to the subject.

109. A method according to paragraph 108, wherein the maintenance doses are administered for at least four weeks from the day of first administration of a maintenance dose to the subject.

110. A method according to paragraph 109, wherein the maintenance doses are administered for at least 12 weeks from the day of first administration of a maintenance dose to the subject.

111. A method according to paragraph 109, wherein the maintenance doses are administered for at least 6 months from the day of first administration of a maintenance dose to the subject.

112. A method according to paragraph 109, wherein the maintenance doses are administered for at least 12 months from the day of first administration of a maintenance dose to the subject.

113. A method according to any of paragraphs to 108 to 112, wherein the maintenance doses are administered for up to 6 years from the day of first administration of a maintenance dose to the subject.

114. A method according to any of paragraphs to 108 to 113, wherein the subject is a human subject.

115. A method according to paragraph 114, wherein the, or each maintenance dose comprises >2,500 IU to 75,000 IU vitamin A per day.

116. A method according to paragraph 114 or 115, wherein the, or each maintenance dose comprises 5,000-75,000, 10,000-75,000, or 20,000-75,000 IU vitamin A per day.

117. A method according to paragraph 115 or 116, wherein the, or each maintenance dose comprises 5,000-50,000, 10,000-50,000, or 20,000-50,000 IU vitamin A per day.

Embodiments of the invention are described below, by way of example only, with reference to the accompanying drawings, in which:

FIG. 1 shows an ultrasonogram of a subtle core lesion in the lateral aspect of superficial digital flexor tendon (SDFT) with generalised surrounding tendonitis of a horse (ID 111112): (a) before administration of any pharmaceutical composition comprising vitamin A; and (b) after daily administration of a pharmaceutical composition comprising vitamin A for 14 days;

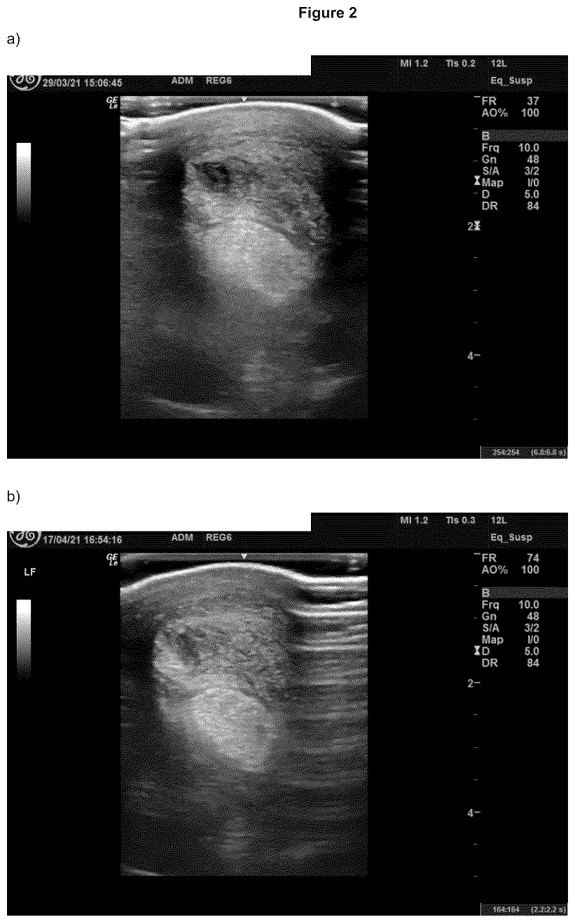

FIG. 2 shows an ultrasonogram of a nasty SDFT core lesion in the medial aspect not quite involving paratenon for a horse (ID REG6): (a) before administration of any pharmaceutical composition comprising vitamin A; and (b) after daily administration of a pharmaceutical composition comprising vitamin A for 14 days;

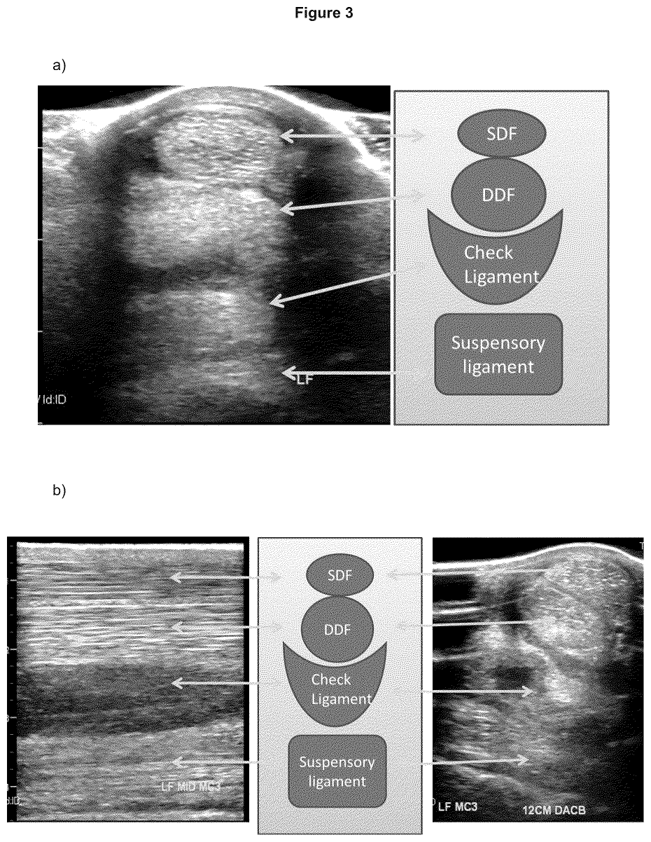

FIG. 3(a) shows a cross-sectional ultrasonogram of the major tendons/ligaments present in the left foot of a horse (normal equine anatomy). FIG. 3(b) shows a longitudinal ultrasonogram (left) of the left foot of a horse compared with a cross-sectional ultrasonogram (right) of the left foot of the same horse (normal equine anatomy);

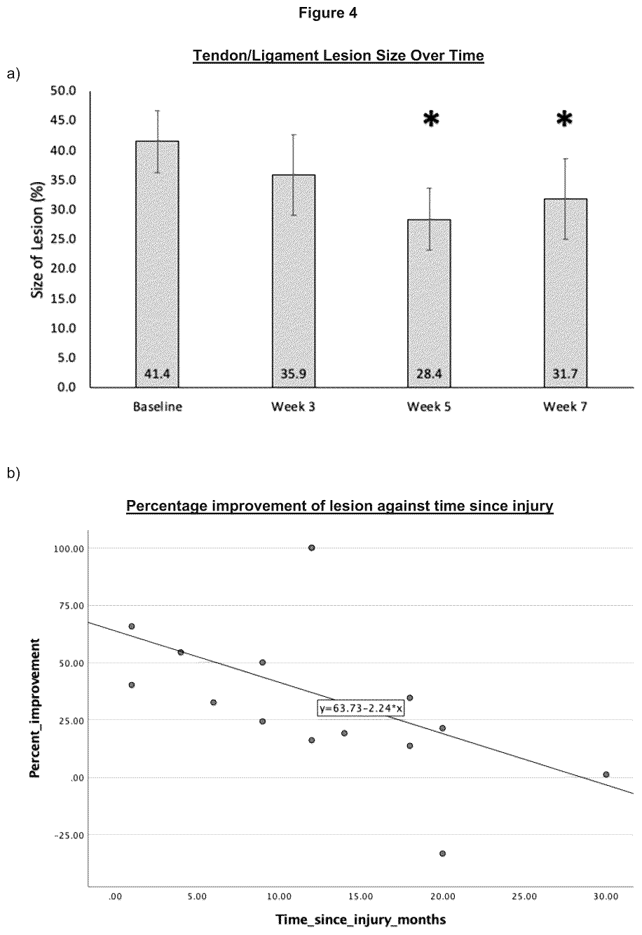

FIG. 4(a) shows a bar graph depicting size of lesion (as a % of baseline lesion size) in tendon/ligament at specified time points after commencing supplementation. Data are presented as mean values, with error bars representing the standard error of the mean. Statistically significant results (p<0.05) compared to baseline were noted with an asterisk (*). Week 3: p=0.169; Week 5: p=<0.001; Week 7: p=0.006. FIG. 4(b) shows scatter plots of percentage improvement (%) against time since injury (months) with all data points included (Pearson's correlation p=0.027);

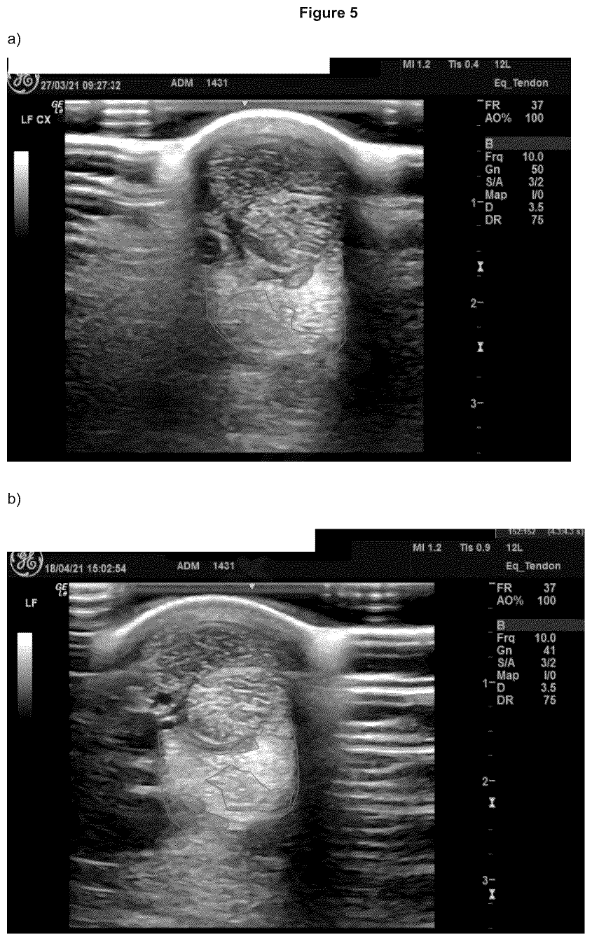

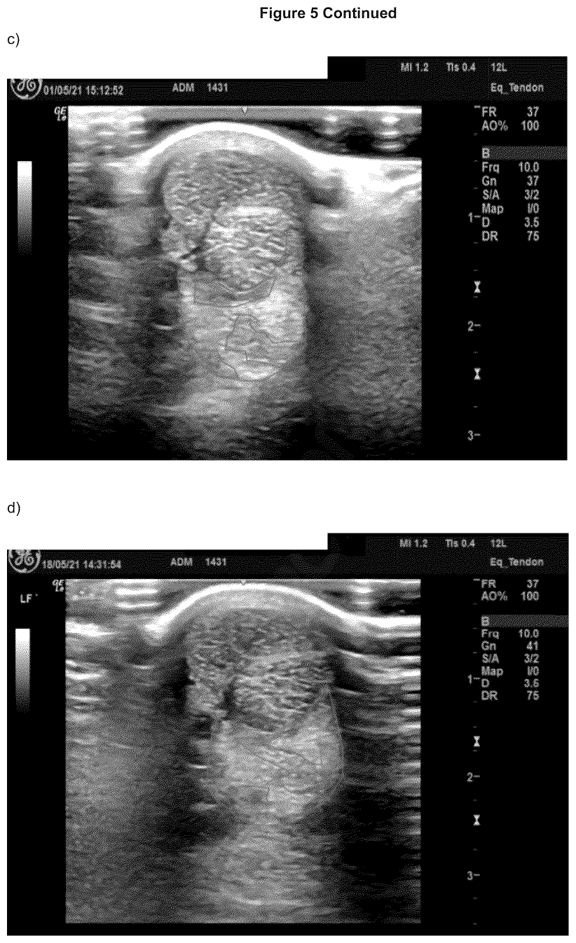

FIG. 5 shows a cross-sectional ultrasonogram of a lesion in the left forelimb check ligament in a horse (ID 1431): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. There is movement artefact shown in the figure;

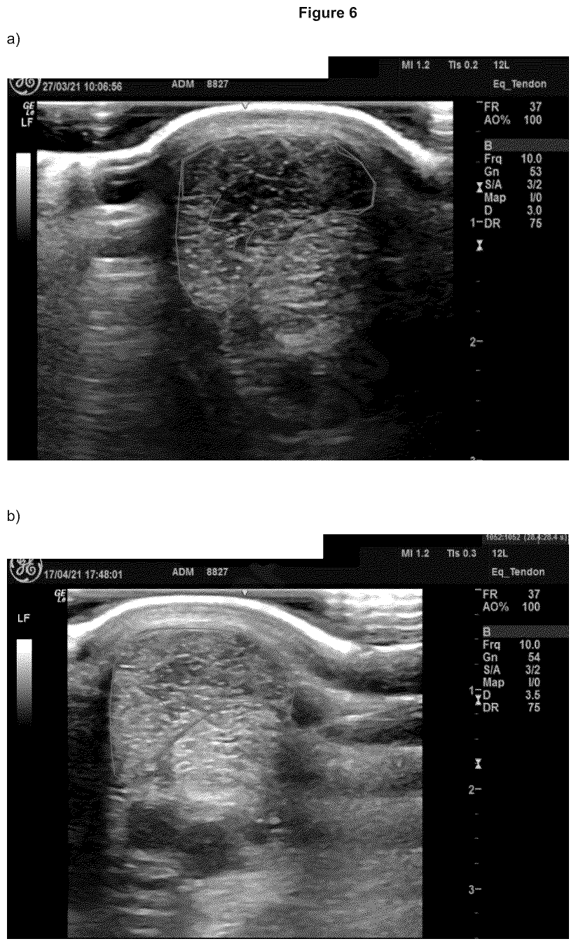

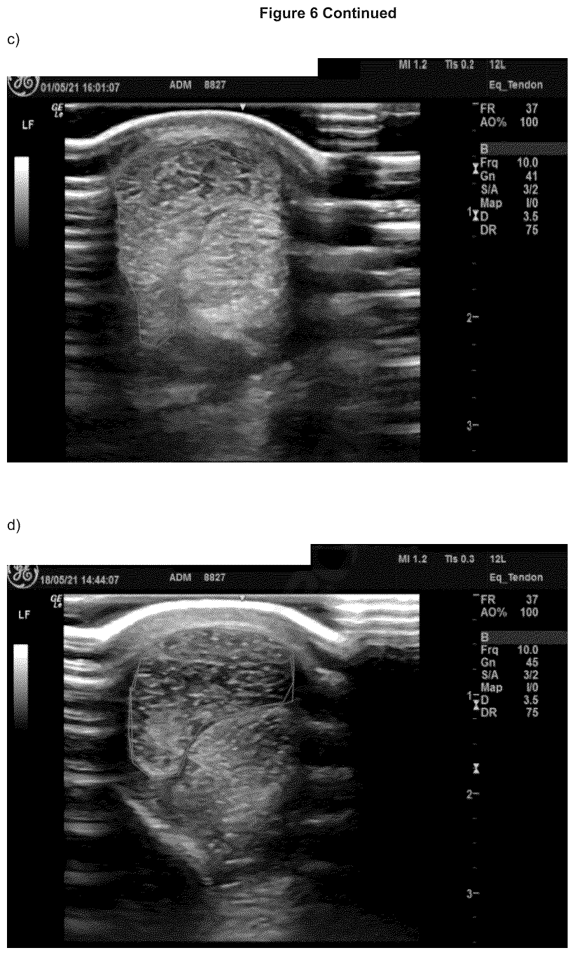

FIG. 6 shows a cross-sectional ultrasonogram of a lesion in the left forelimb SDFT in a horse (ID 8827): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

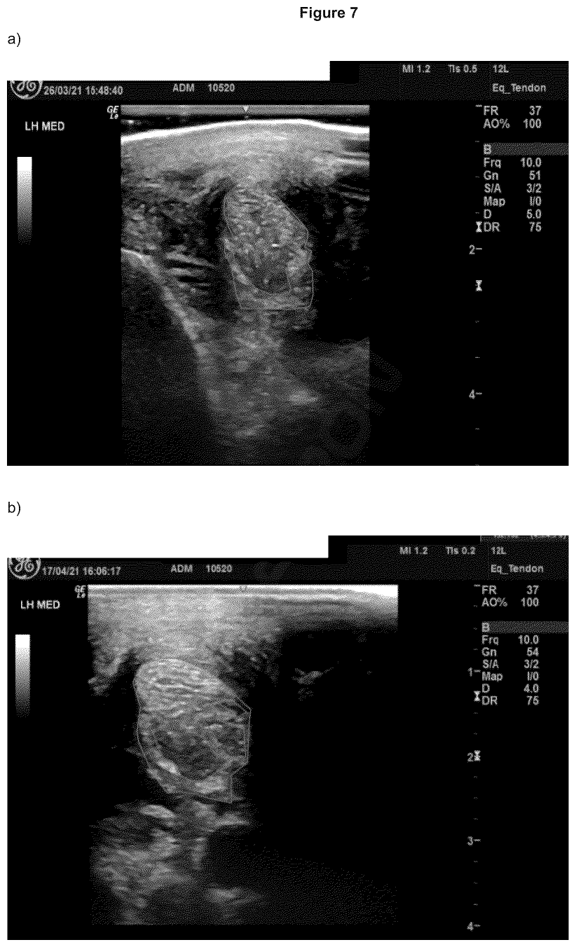

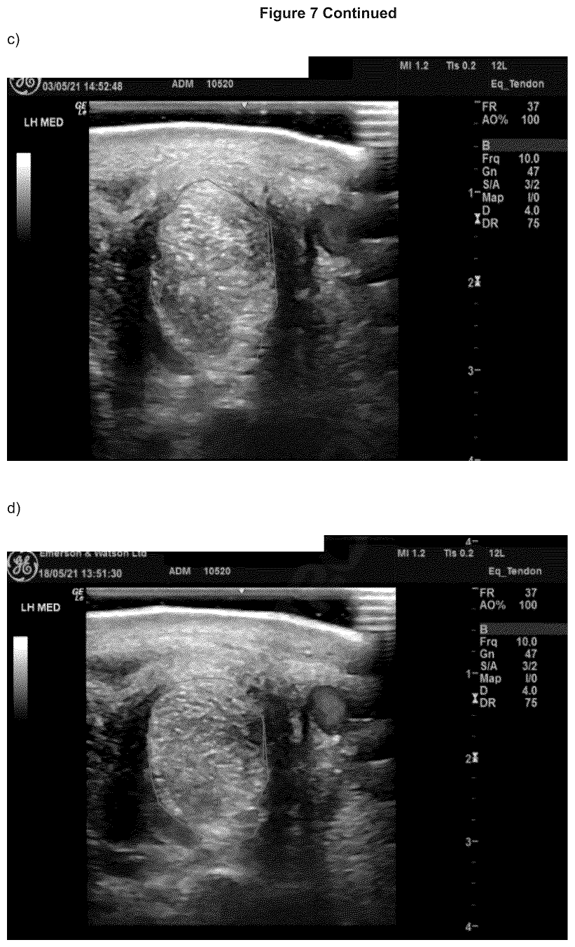

FIG. 7 shows a cross-sectional ultrasonogram of a lesion in the left hindlimb medial suspensory branch ligament in a horse (ID 10520): (a) before administration of any vitamin

A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. (d) shows improvements in axial aspect of the branch;

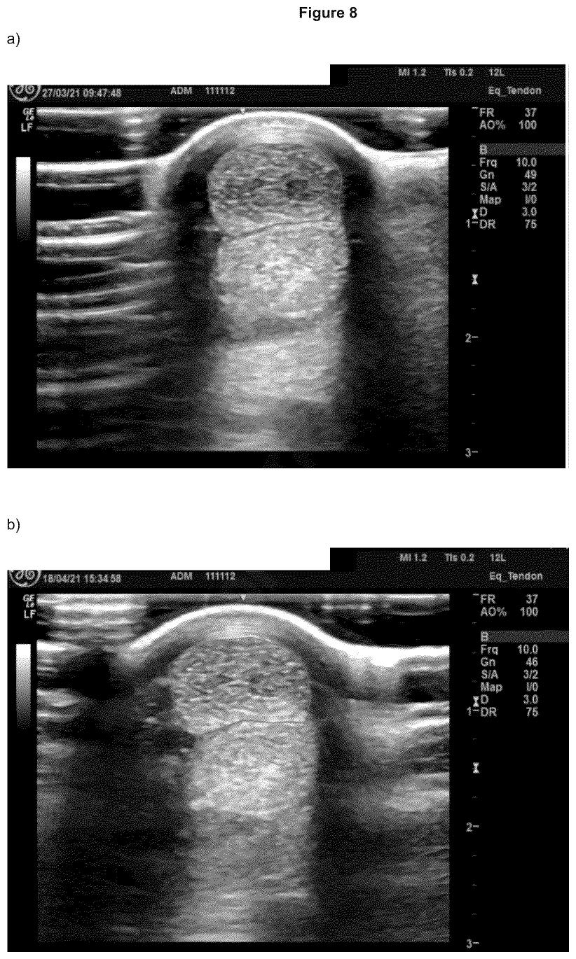

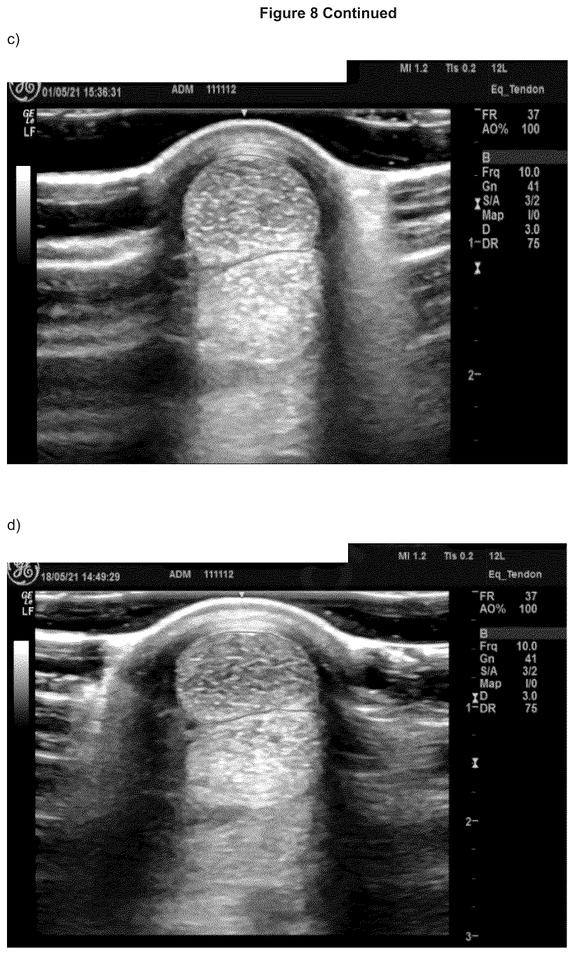

FIG. 8 shows a cross-sectional ultrasonogram of a lesion in the left forelimb SDFT in a horse (ID 111112): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. Some filling in of lesion in lateral aspect of tendon;

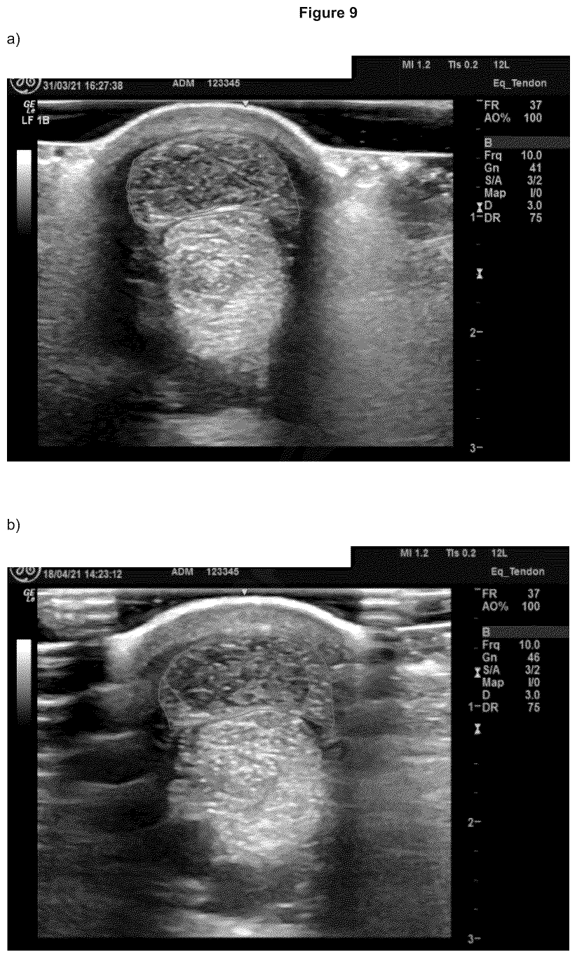

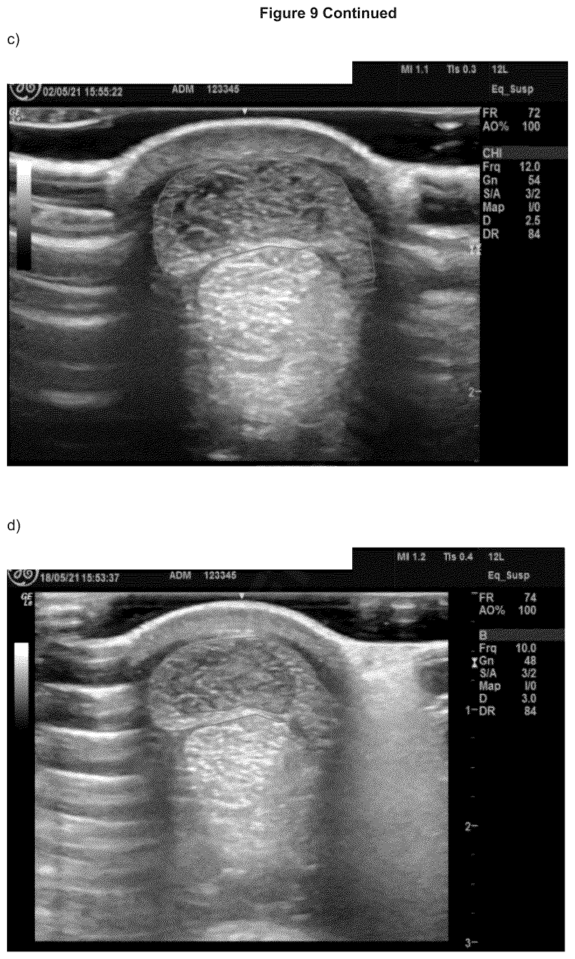

FIG. 9 shows a cross-sectional ultrasonogram of a lesion in the left forelimb SDFT in a horse (ID 123345): (a) before administration of any vitamin A supplement comprising vitamin A; (b) after daily administration of a pharmaceutical composition comprising vitamin

A for 3 weeks; (c) after daily administration of a pharmaceutical composition comprising vitamin A for 5 weeks; and (d) after daily administration of a pharmaceutical composition comprising vitamin A for 7 weeks;

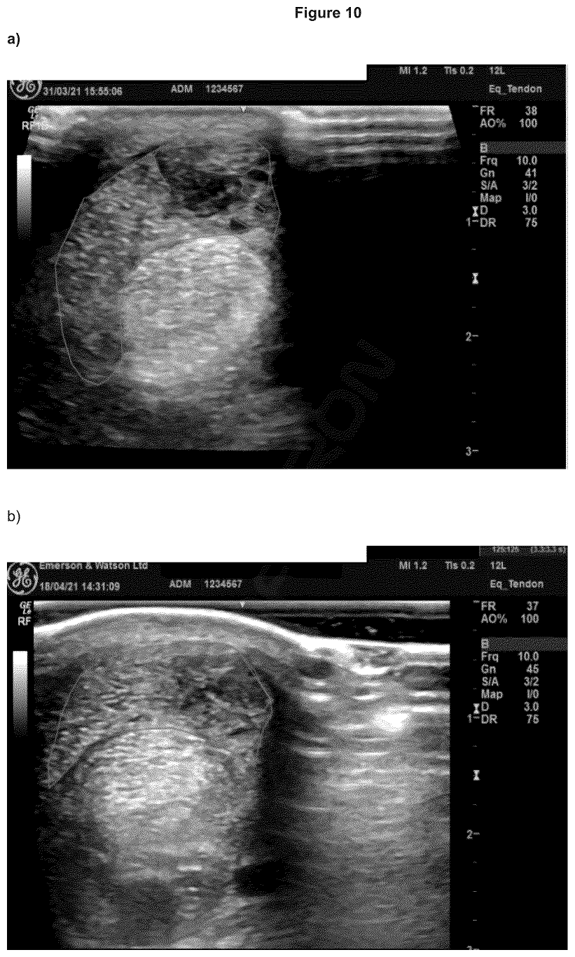

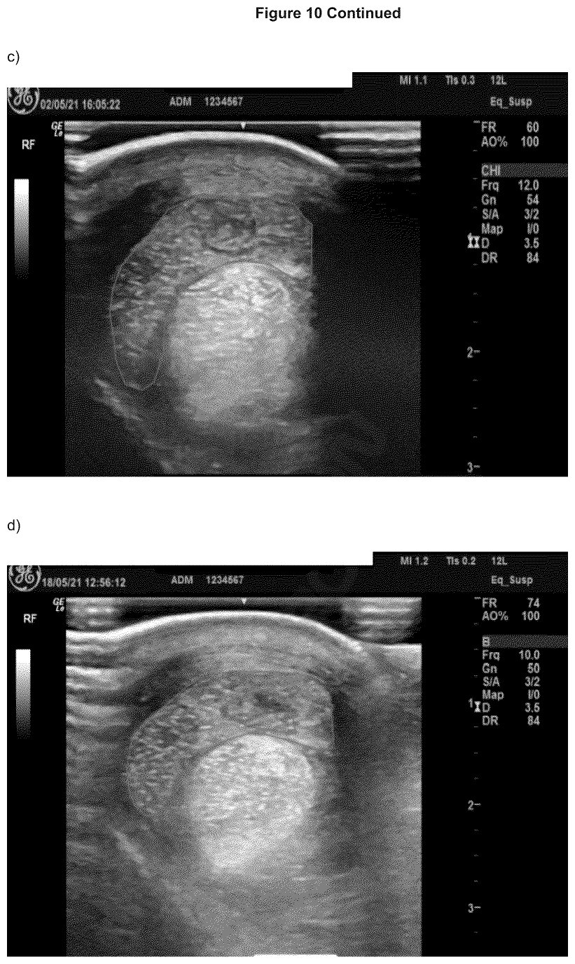

FIG. 10 shows a cross-sectional ultrasonogram of a lesion in the right forelimb lateral SDFT in a horse (ID 1234567): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. Tendon is clearly filling in well;

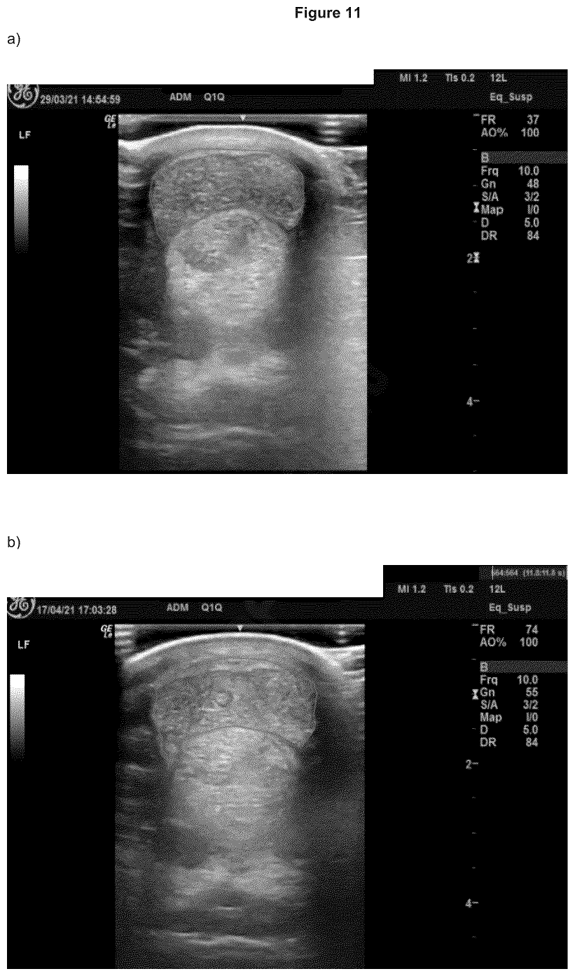

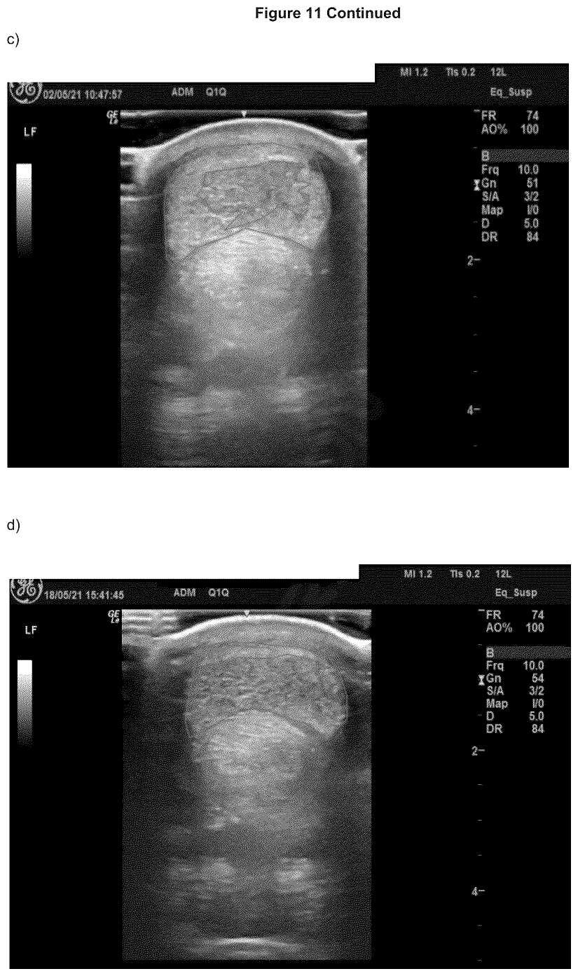

FIG. 11 shows a cross-sectional ultrasonogram of a lesion in the left forelimb SDFT in a horse (ID Q1Q): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

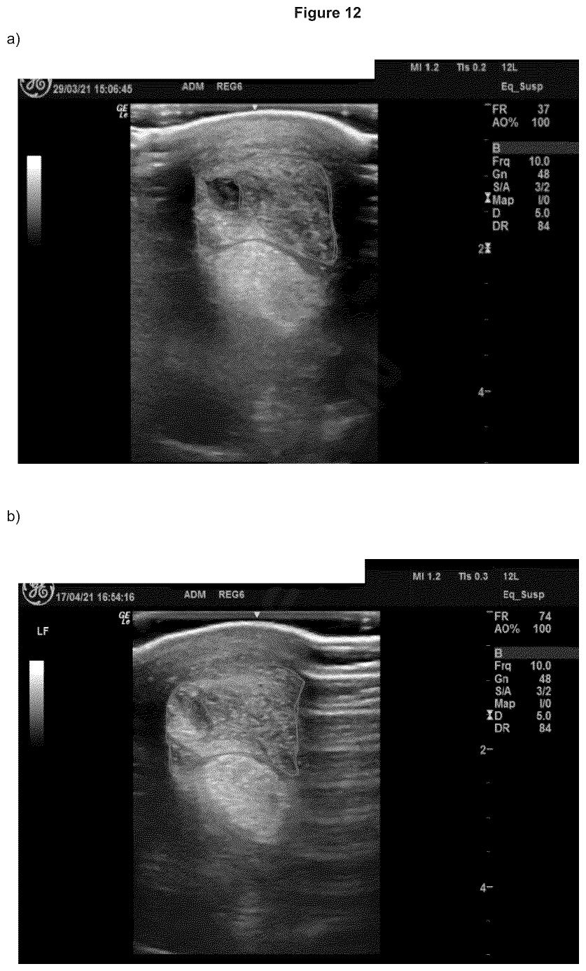

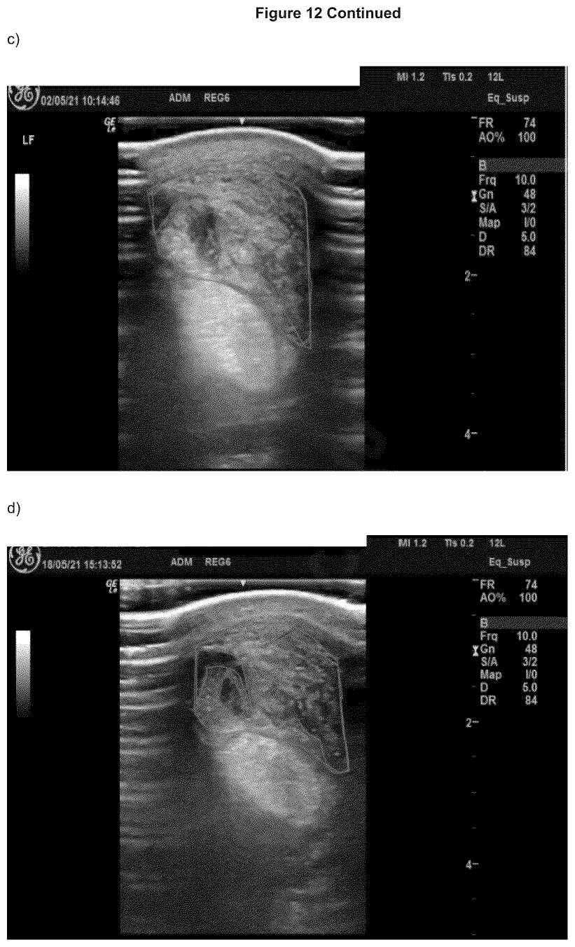

FIG. 12 shows a cross-sectional ultrasonogram of a lesion in the left forelimb SDFT in a horse (ID REG6): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks;

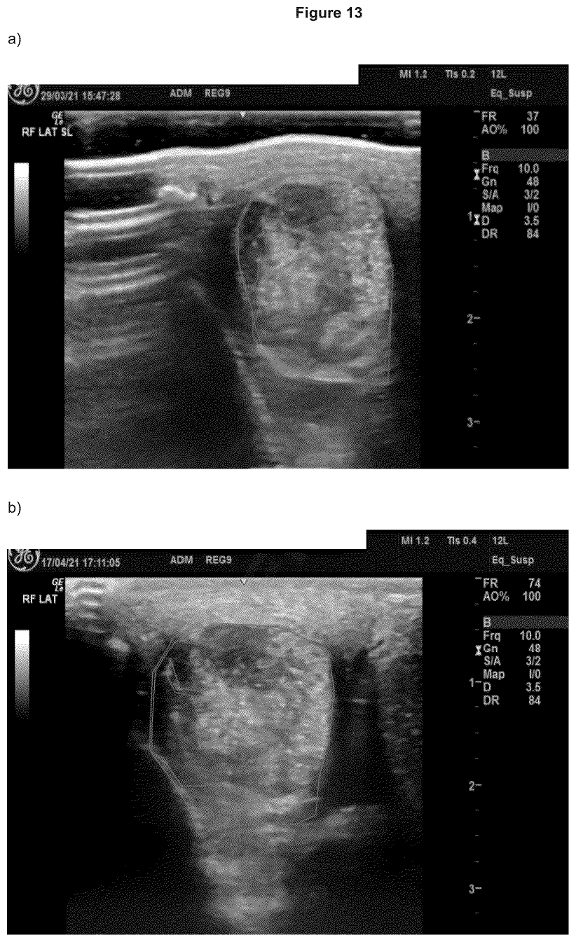

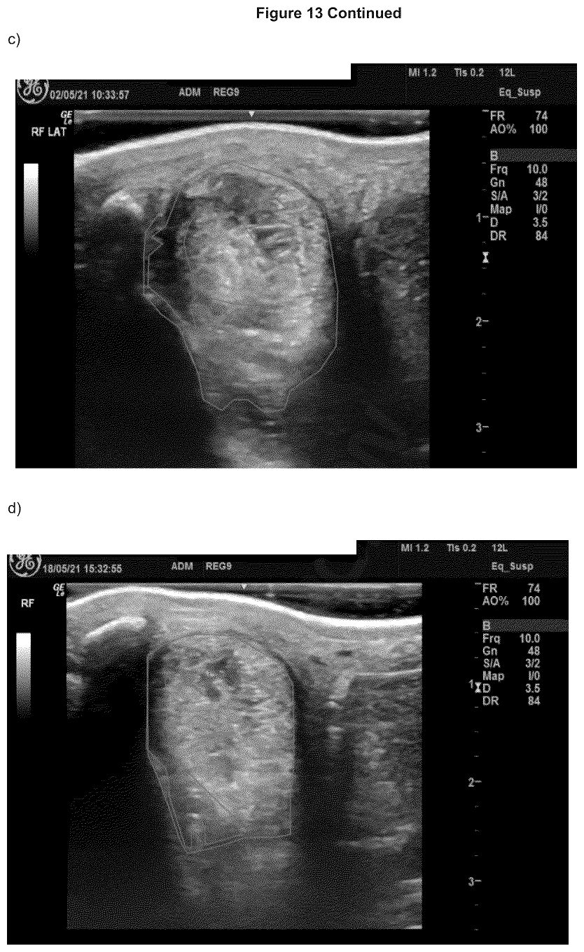

FIG. 13 shows a longitudinal ultrasonogram of a lesion in the right forelimb lateral suspensory branch ligament in a horse (ID REG9): (a) before administration of any vitamin A supplement comprising vitamin A; (b) after daily administration of a pharmaceutical composition comprising vitamin A for 3 weeks; (c) after daily administration of a pharmaceutical composition comprising vitamin A for 5 weeks; and (d) after daily administration of a pharmaceutical composition comprising vitamin A for 7 weeks. Image at week 7 (FIG. 12(d)) shows improvement of injury as deeper lesion less visible.

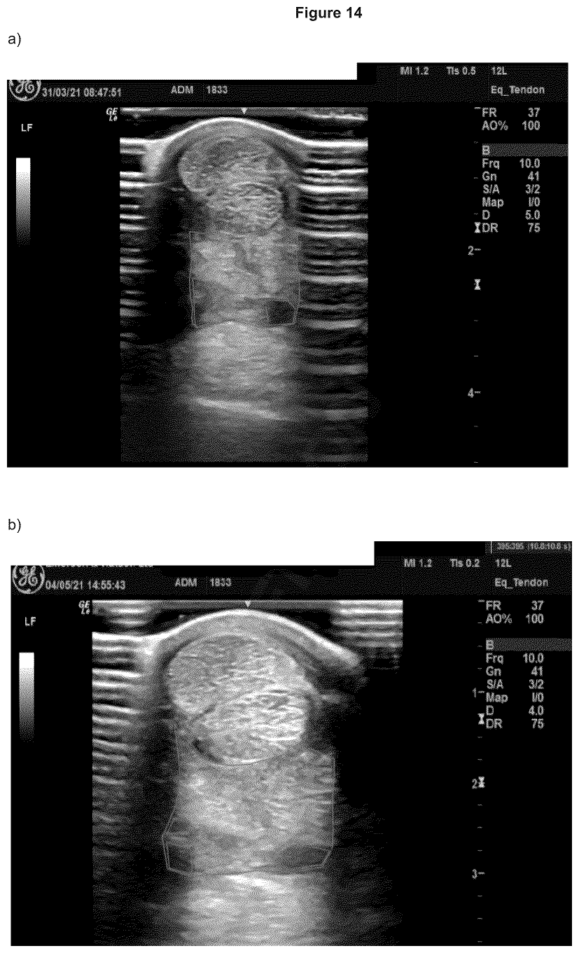

FIG. 14 shows a cross-sectional ultrasonogram of a lesion in the left forelimb check ligament in a horse (ID 1833): (a) before administration of any vitamin A supplement comprising vitamin A; and (b) after daily administration of a pharmaceutical composition comprising vitamin A for 5 weeks. Some improvement of check ligament injury appearance. The horse also received platelet rich plasma;

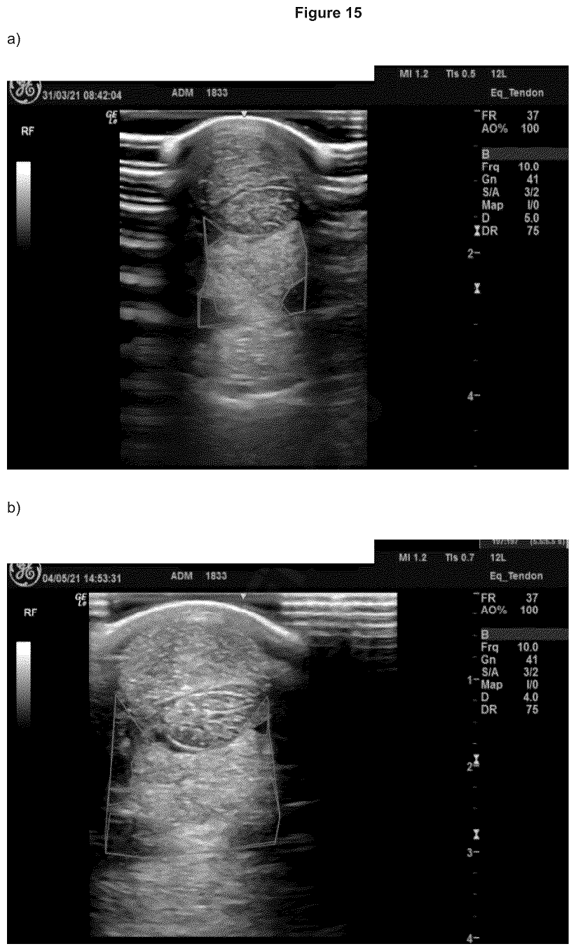

FIG. 15 shows a cross-sectional ultrasonogram of a lesion in the right forelimb check ligament in a horse (ID 1833): (a) before administration of any vitamin A supplement comprising vitamin A; and (b) after daily administration of a pharmaceutical composition comprising vitamin A for 5 weeks;

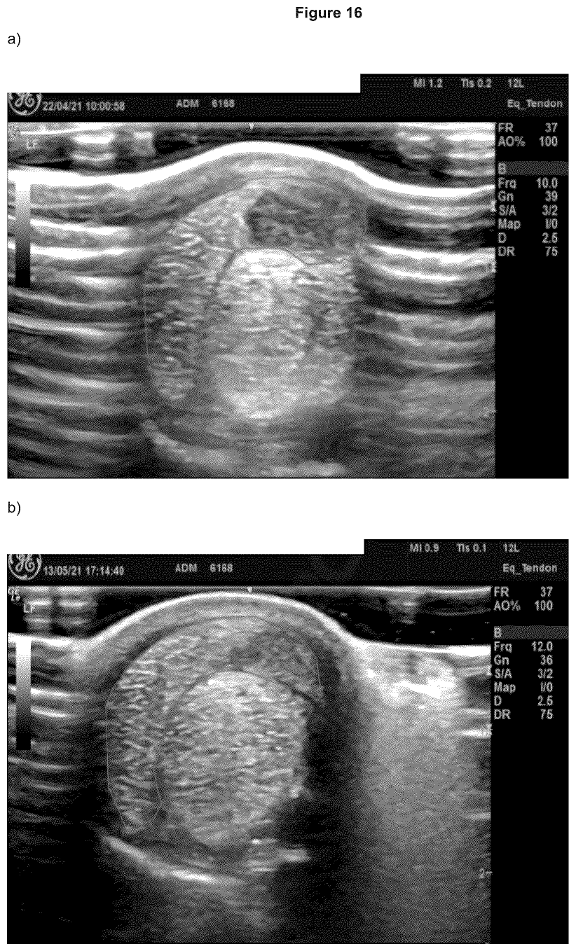

FIG. 16 shows a cross-sectional ultrasonogram of a lesion in the left forelimb lateral SDFT in a horse (ID 6168): (a) before administration of any vitamin A supplement comprising vitamin A; and (b) after daily administration of a pharmaceutical composition comprising vitamin A for 3 weeks. The figure shows good infilling of lateral SDFT lesion.

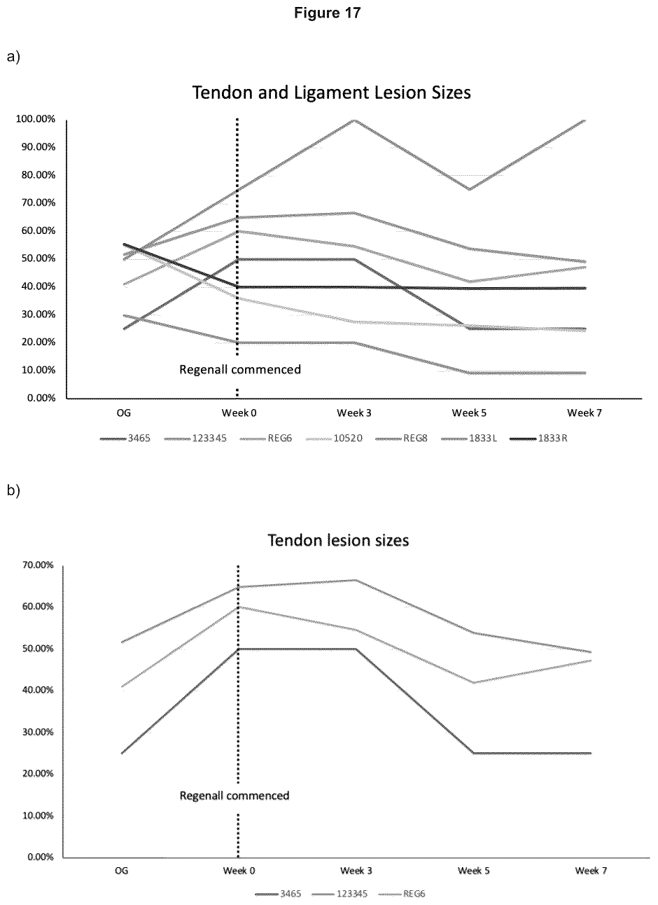

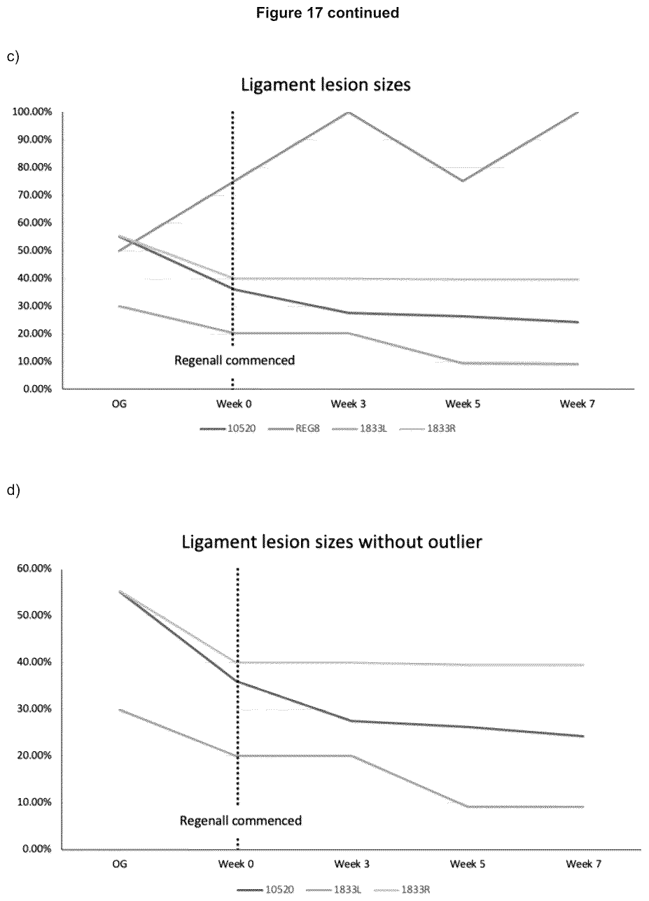

FIG. 17 shows line graphs depicting size of lesion (as a % of baseline lesion size) in tendon/ligament injuries at specified time points after original injury (OG): (a) shows tendon/ligaments as a single cohort; (b) shows tendon injuries as a separate, single cohort; (c) shows ligamentous injuries as a separate, single cohort; and (d) shows ligamentous injuries as a separate, single cohort with outlier removed from data set. Data are presented as actual values for each subject;

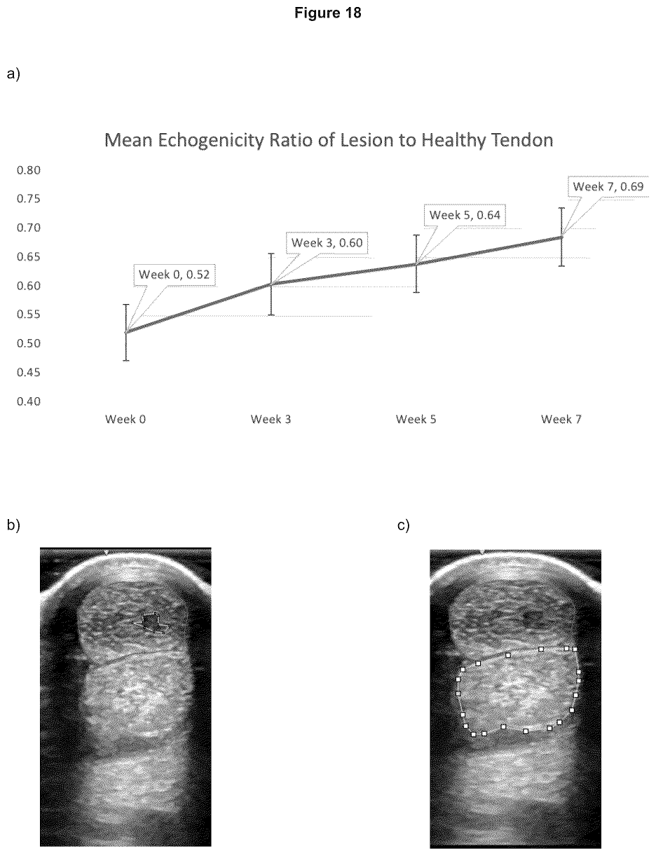

FIG. 18(a) shows a line graph depicting the mean echogenicity ratio of a tendon lesion to an adjacent healthy tendon at specified time points after commencing supplementation. Data are presented as mean values for the tissues, with error bars representing the standard error of the mean. A value of 1 would indicate perfect regeneration of native tendon. FIG. 18(b) shows a cross-sectional ultrasonogram showing the outline of the tendon lesion of (a) with the injured tendon also outlined. FIG. 18(c) shows a cross-sectional ultrasonogram showing the outline of an adjacent healthy tendon used as a comparison tissue to calculate the echogenicity ratio, as well as the outline of the lesion and injured tendon;

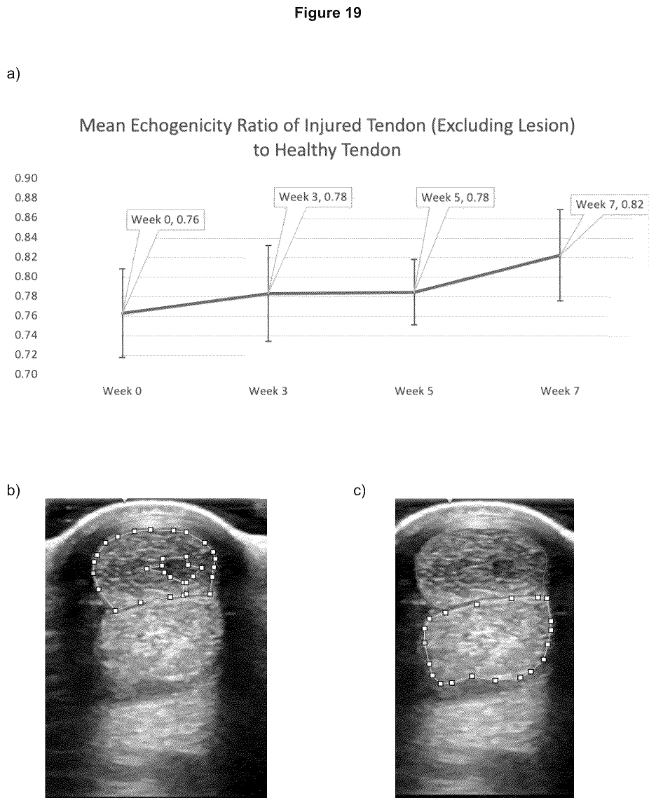

FIG. 19(a) shows a line graph depicting the mean echogenicity ratio of an injured tendon (with the area of lesion excluded) to an adjacent healthy tendon at specified time points after commencing supplementation. Data are presented as mean values for the tissues, with error bars representing the standard error of the mean. A value of 1 would indicate perfect regeneration of native tendon. FIG. 19(b) shows a cross-sectional ultrasonogram showing the outline of the tendon lesion of (a) with the injured tendon also outlined, the lesion is excluded from the area of injured tendon for analysis. FIG. 19(c) shows a cross-sectional ultrasonogram showing the outline of an adjacent healthy tendon used as a comparison tissue to calculate the echogenicity ratio, as well as the outline of the lesion and injured tendon;

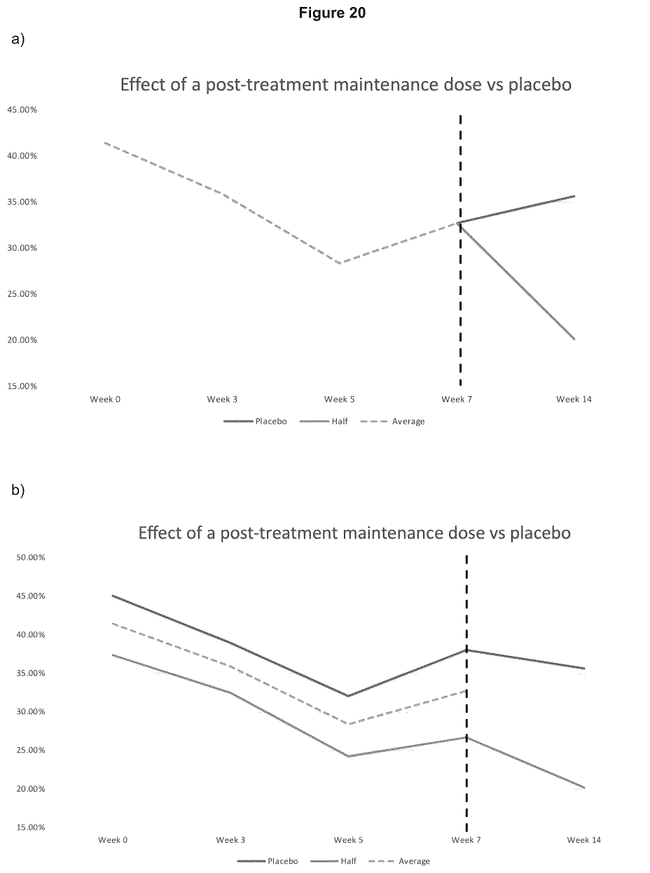

FIG. 20 shows a line graph depicting the effect of post-treatment maintenance dose of vitamin A supplement on the size of tendon/ligamentous lesions: (a) shows mean lesion size from week 0 to week 7 on full-treatment doses of vitamin A supplement before splitting into the values of the maintenance dose and placebo groups for week 7 to week 14; and (b) shows the lesion size for both maintenance and placebo groups from week 0 through to week 14, as well as the mean values for the groups from week 0 to week 7;

FIG. 21 shows a series of cross-sectional ultrasonograms of a tendon injury that became re-injured after administration of full-treatment doses of vitamin A supplement was stopped and a maintenance dose of supplement was administered. Figure (a) shows an ultrasonogram of the lesion at baseline (week 0) before treatment with vitamin A supplement commenced; (b) shows an ultrasonogram at week 7 of administration with full-treatment doses of vitamin A supplement; and (c) shows ultrasonogram of the lesion at week 14 (after 7 weeks of post full-treatment maintenance doses of vitamin A supplement); and

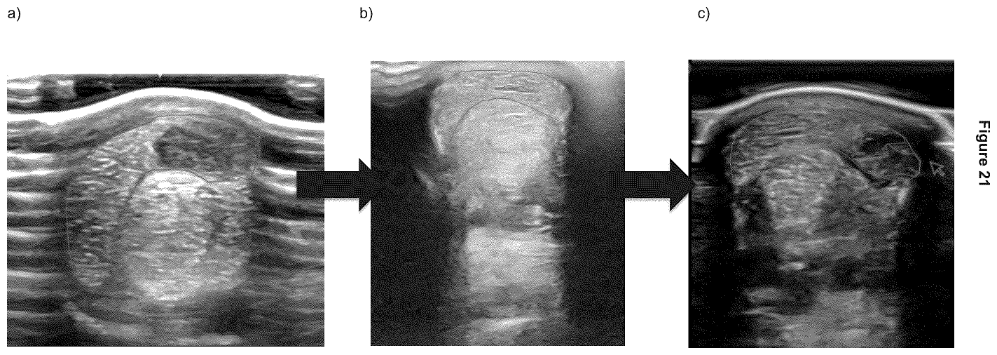

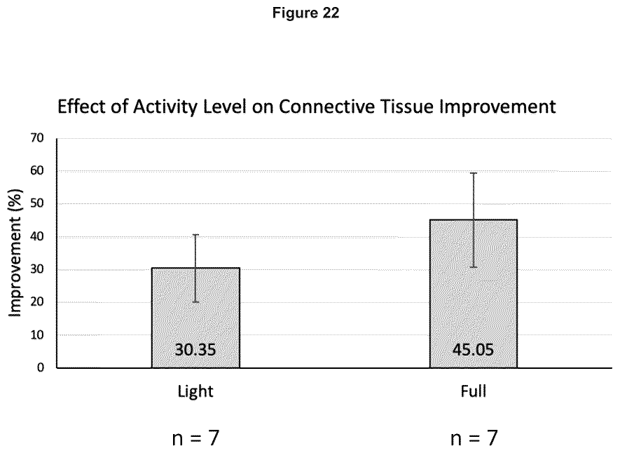

FIG. 22 shows a bar graph depicting the effect of activity level on connective tissue improvement in horses on vitamin A supplement. Improvement is shown as % improvement from baseline. Data are presented as mean values, with error bars representing the standard error of the mean.

FIG. 23 shows a schematic view of IPF pathogenesis:

Repeated injuries over time lead to maladaptive repair process, characterized by AEC2s apoptosis, proliferation and epithelium-mesenchymal cross-talk (a) and following fibroblasts, myofibroblasts proliferation and accumulation of extracellular matrix (b). (abbreviations: CCL2: chemokine C-C motif ligand 2; CXCL12: C-X-C motif chemokine 12; FGF: fibroblast growth factor; PAI-1: plasminogen activator inhibitor 1; PAI-2: plasminogen activator inhibitor 2; PDGF: platelet-derived growth factor; TGF-1: Transforming Growth Factor-Beta 1; TNF-α: tumor necrosis factor-alpha; VEGF: vascular endothelial growth factor). From Sgalla, et al. Idiopathic pulmonary fibrosis: pathogenesis and management. Respir Res 19, 32 (2018), distributed under the terms of the Creative Commons Attribution 4.0 International License (http://creativecommons.org/licenses/by/4.0/) (original figure in colour);

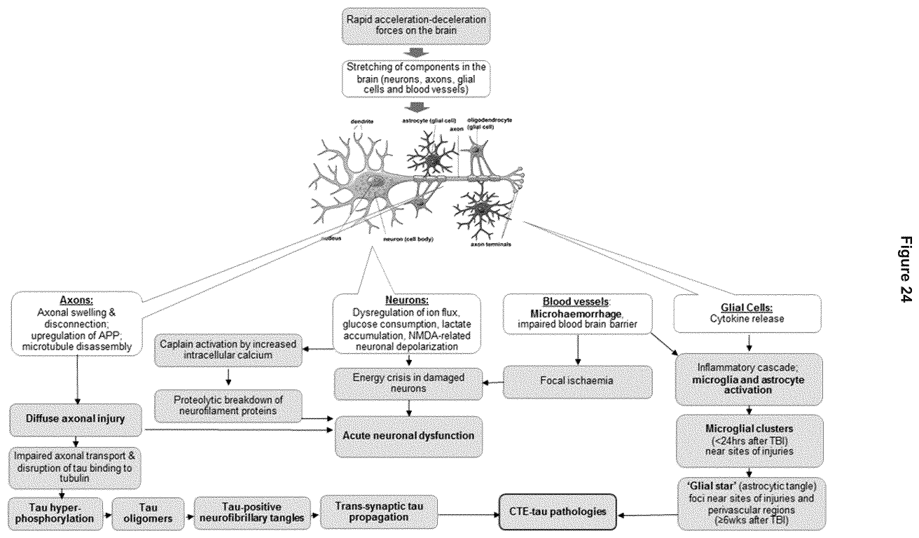

FIG. 24 shows a schematic illustration of the proposed cascade of events triggered by acute TBIs and its possible mechanistic links with the development of CTE pathology (from Ling et al., “Neurological consequences of traumatic brain injuries in sports”, Molecular and Cellular Neuroscience 66 (2015) 114:122).

### EXAMPLES

### Example 1

Tendon Injury—Recovery Following Treatment with Vitamin A

| | |
|---|---|
| Patient | Horse subject 1 (polo pony) |
| Age | 14 years |
| Sex | Mare |
| Health status | Good |

### Injury

| | |
|---|---|
| Type of injury | Tendon injury |
| When the injury occurred | 14 Feb. 2019 |
| Cause of the injury | Hyperextension and/or blunt trauma |
| Tissue(s) affected | Tendon |
| Impairments resulting from the | Lameness and loss of athletic |
| injury | function |

Treatment with Conventional Methods

Single dose 30 mg dexamethasone IV to treat the initial acute inflammation followed by 1 g phenylbutazone PO BID for 5 days. Ice applied to affected area for 15 minutes twice a day for 5 days.

Extent of Recovery Following Treatment with Conventional Methods

Recovery has progressed as expected with these types of lesions in horses. Moderate lameness, heat and pain response to firm digital palpation improved rapidly over the first 7 to 10 days. The horse then started a programme of incremental walking exercise.

Treatment with Vitamin A
How soon after the injury was treatment was treatment with Vitamin A initiated?
Treatment with Vitamin A was initiated approximately 20 days after the initial injury.
Type of Vitamin A administered
Retinyl palmitate (93.8% Retinol Equivalent)
Route of administration
Oral
Dose of Vitamin A per administration
750,000 IU
Frequency of administration
Twice daily (1,500,000 IU per day)
Period of administration
Continuing (at least 4 weeks)
   Recovery Following Treatment with Vitamin A

Functional recovery has been good considering the severity of the lesion. Ultrasonography has confirmed good reduction in the size of the lesion (notable reduction in size of anechoic pockets of interstitial haemorrhage). Horses are prey species and often do not show signs of overt pain or lameness even with severe lesions. It is not uncommon for such lesions to take between 12 to 18 months to heal.

### Example 2: Patient-Reported Outcome Measure

Spinal Cord Injury—Recovery Following Treatment with Vitamin A

| | |
|---|---|
| Patient | Human Subject 1 |
| Age | 68 |
| Sex | Male |
| Health status | Good |

### Injury

| | |
|---|---|
| Type of injury | Spinal cord injury |
| When the injury occurred | November, 1963 |
| Cause of the injury | Trauma to T4 |
| Tissue(s) affected | Left lateral spinothalamic tract (Brown-Sequard |
| | syndrome) |
| Impairments resulting | Left stiff knee, right lower back pain, difficulty |
| from the injury | walking |

Treatment with Conventional Methods
Laminectomy T4 and T5
Baclofen 20 mg AM and 30 mg PM for the past 13 years.
   Extent of Recovery Following Treatment with Conventional Methods

Seven years after my operation (1963) I was very active and had a Brown Belt in Tae Kwon Do, I started to limp when I was 24 years old. At 38 years I could not run. At 52 my limp was more pronounced and I had a series of Botox injections to my left leg and thigh muscles over a period of 2-3 years. At 55 I was started on Baclofen and currently I take 20 mg every morning and 30 mg at night. At 64 my mobility continues to decrease and I began using a cane to ambulate. I began to experience pain of my right lower back approximately 4 years ago. It ranges from a scale of 2-6/10 depending on activities I do as well as the weather.

Treatment with Vitamin A
How soon after the injury was treatment was treatment with Vitamin A initiated?
56 years
Type of Vitamin A administered
Vitamin A 50,000 IU (from Retinyl palmitate and fish liver oil)
Route of administration
Oral
Dose of Vitamin A per administration
50,000 IU
Frequency of administration
Once daily
Period of administration
3 months
   Recovery Following Treatment with Vitamin A

The pain is approximately 10-15% less and when I have it, the duration is much less. The weather conditions have to be severe before I begin to experience pain. Previously I could forecast if it was going to rain a day or two ahead because my right lower back would start to ache. Now the weather conditions have to be more severe for my back to start aching.

### Example 3: Patient-Reported Outcome Measure

Spinal Cord Injury—Recovery Following Treatment with Vitamin A

| | |
|---|---|
| Patient | Human Subject 2 |
| Age | 69 |
| Sex | Male |

### Injury

| | |
|---|---|
| Type of injury | Spinal cord injury |
| When the injury occurred | Beginning of September 2016 |
| Cause of the injury | Spinal stenosis |
| Tissue(s) affected | Paralyzed both legs and no feeling up |
| | to chest |
| Impairments resulting from | Both legs paralyzed and up to the chest |
| the injury | |

Treatment with Conventional Methods

First surgery 22^{nd} of September 2016, T3 to T4 (Considerable bleeding occurred during the operation). Second surgery in two places (laminotoimiu) on 9th of March 2017, Th10-Th12 and L1-L5.

Drugs: After surgery 2016: Paracetamol 1 g×4, Codein 30 mg×4, Valsartan 82.5×1, Atorvastatin 40 mg×1. Omeprazol 20 mg×1, D3 vitamin 2000 ae×1, Kaleorid 750 mg×1, Klexane 40 mg×1, Betolvex 1 mg×1, Valsartan/hydrochlortiazide 80/12.5 ×1, Atacor 40 mg×1, Gabapentin 300 plus 900 mg, Zopiclone 7.5 mg vesp. Tradolan 50 mg 2×2, 2019: Losartan/Hydrochlorothiazide 100 mg/25 mg ×1, Acetylaslisylsyre 75 mg×1, Diclomex Rapid 50 mg 1×3, Anti Leg Cramps (NAVEH) 2×1, Vitamin A10.000×5. Imovane 7.5 mg 1×1. Milk Thistle (Lamberts) 8500 mg of seed-Providing Silymarin 200 mg 1×1.

Extent of Recovery Following Treatment with Conventional Methods

Daily exercises from the beginning. Wheelchair and crutches. Cramps in legs, need to take Gapabentin 3-5 pr day and Tradolan 2 pr day.

Treatment with Vitamin A
How soon after the injury was treatment was treatment with Vitamin A initiated?
Starting in October 2018. For three months taking vitamin A 10,000×3. Increasing in end January 2019 10,000×5.
Type of Vitamin A administered
Vitamin A (from cod liver oil and vitamin A palmitate)
Route of administration
Oral
Dose of Vitamin A per administration
Initially total of 30,000 IU; From end of January 2019 total of 50,000 IU; 3,000 mcg RAE
Frequency of administration
For 3 months 3 tablets a day and for 3 months 5 tablets a day
Period of administration
6 months
   Recovery Following Treatment with Vitamin A

First I found recovery more walking control and balance. Coordination was better. In mid-January 2019 a big pain in left leg and ankle and could not train my left legs. My doctor said it was probably because of one of the pockets with water drops managed to get into my spinal cord. I asked him if that could happen again, he said no, may be only 10% possibility. This problem took me 1.5 months to recover. Now in March 2019 I feel much stronger, more balanced, more movement control and I am walking as a whole 6×42 steps (up and down) twice in a week. In beginning of April start walking without any support on even level 30-50 meters with better controlling.

Subject provided videos showing the subject walking along an even level with very little to no support and walking up and down a set of stairs.

### Example 4: Patient-Reported Outcome Measure

Spinal Cord Injury—Recovery Following Treatment with Vitamin A

| | |
|---|---|
| Patient | Human Subject 3 |
| Age | 79 |
| Sex | Male |
| Health status | Good |

### Injury

| | |
|---|---|
| Type of injury | Disc/nerve pain |
| When the injury occurred | 2018 |
| Tissue(s) affected | Facet joint |
| Impairments resulting from the injury | Pain around knee (limping) |

Treatment with Conventional Methods
Injections to disc/nerve×2
   Treatment with Vitamin A
How soon after the injury was treatment with Vitamin A initiated?
3/4 months
Type of Vitamin A administered
Vitamin A 50,000 IU
Route of administration
Oral
Dose of Vitamin A per administration
50,000:1
Frequency of administration
Daily
Period of administration
4-5 months
   Recovery Following Treatment with Vitamin A

Vitamin A has improved pain level which has almost disappeared×80%.

### Example 5: Patient-Reported Outcome Measure

Spinal Cord Injury—Recovery Following Treatment with Vitamin A

| | |
|---|---|
| Patient | Human Subject 4 |
| Age | 60 |
| Sex | Male |

### Injury

| | |
|---|---|
| Type of injury | Spinal cord injury and now have lumbar spinal |
| | stenosis as well |
| When the injury | Spinal Cord Injury in March 1998 at C4/C5 level |
| occurred | |
| Cause of the injury | Fell off mountain in France skiing |
| Tissue(s) affected | Incomplete injury, initial paralysis below C4/C5 |
| | but reasonable mobility has been recovered. All |
| | tissues effected below C4/C5 |
| Impairments resulting | Loss of some mobility below C4/C5 level, I walk |
| from the injury | with a stick. |

Treatment with Conventional Methods

In 1998 I had a laminectomy after the accident in France, and then a disc fusion in London both at C4/C5 level.

In 2009 I had a further disc replacement in my neck after a fall.

I had injections in my neck in May 2018 for stenosis and severe neck pain.

I have had injections in my lumbar region in May and September 2018 and March 2019 and rhizolysis in the lumbar region in September 2018 and at 3 levels in March 2019.

I have intermittently taken pain killers including ibuprofen, paracetomol and naproxen. In February 2019 I took Arcoxia for 4 weeks. I also take antibiotics as a prophylaxis to prevent UTIs. I also take toltoredine, topsium chloridfe, senna and diactyl.

Extent of Recovery Following Treatment with Conventional Methods

Good recovery from the original spinal cord injury in my neck but it left me with very impaired mobility and walking with a stick.

After the injection in my neck in May 2018 this significantly helped remove the pain but it left me with a very stiff neck which still ached quite a lot but the pain was bearable.

Improved mobility since the injection and rhizolysis in lumbar region in March 2019.

Treatment with Vitamin A

How soon after the injury was treatment was treatment with Vitamin A initiated? 21 years later in February 2019

Type of Vitamin A Administered
Vitamin A (from cod liver oil and vitamin A palmitate)
Route of administration
Oral
Dose of Vitamin A per administration
Total of 50,000 IU (5×10,000 IU tablets)
Frequency of administration
I take 50,000 IU in one administration in the morning daily
Period of administration
Continuing: Almost 2 months
   Recovery Following Treatment with Vitamin A

Very soon after taking Vitamin A my neck was much more flexible and the aching largely disappeared. I have much more movement in my neck and the aching has now disappeared.

Big improvement in overall mobility, walking and feeling of better health following the rhizolysis in early March 2019—I do not know if this all due to the procedure or whether this improvement is due to the Vitamin A. but the big improvement in my neck before the procedure was after taking Vitamin A and continued after I stopped taking the Arcoxia.

### Example 6—Treatment of Equine Tendon Injury

This example describes the effect of a pharmaceutical composition comprising vitamin A in treating tendon injury in horses.

Tendon injuries result in the formation of a fibrovascular scar that never attains the characteristics of normal tendon. Tendon healing is characterised by the formation of fibrovascular scar tissue, as tendon has very little intrinsic regenerative capacity. The molecular mechanisms resulting in scar tissue formation after tendon injuries are not well understood (as reviewed in Schneider et al. Rescue plan for Achilles: Therapeutics steering the fate and functions of stem cells in tendon wound healing; Advanced Drug Delivery Reviews 129 2018 352-375). Briefly, in the first few days after injury a blood clot forms that serves as a preliminary scaffold for invading cells followed by a more robust vascular network which is essential for the survival of tenocytes engaged in the synthesis of new fibrous tissue. Thereafter, fibroblasts are recruited to the injured site and produce initially disorganised extracellular matrix components. Following this, a remodelling stage commences characterised by tissue changes resulting in a more fibrous appearance and eventually a scar-like tendon tissue can be observed.

Current biologic treatment strategies have not achieved tendon regeneration but include the use of extracellular matrix patches to provide a scaffold for new cell growth and differentiation (as reviewed in Galatz et al. Tendon Regeneration and Scar Formation: The Concept of Scarless Healing, J. Orthop. Res. 2015, 33(6) 823-831). Platelet rich plasma which comprises a multitude of growth factors normally involved in repair processes has also been investigated for use in tendon repair. However, there is no evidence that either strategy induces tendon regeneration. Tendon injuries are also a particular problem in horses.

Tendon injury has a similar pathophysiology to injury in other tissues (including spinal cord injury) in that they may be characterised by excessive deposition of scar tissue. Evidence for an effective treatment of tendon injury (including evidence for inhibition of scar tissue formation following tendon injury) is considered to provide evidence also for an effective treatment of other types of injury (including spinal cord injury), for example, through inhibition of scar tissue formation.

### Pharmaceutical Composition Used:

Vitamin A palmitate (also known as preformed vitamin A, or retinyl palmitate) mixed with coconut oil to provide a final vitamin A concentration of 10,000 IU/ml.

### Administration of Pharmaceutical Composition:

Horses with tendon injury were orally administered vitamin A palmitate mixed with coconut oil, at a dose of 160,000 IU once per day for 14 days.

### Results

Ultrasonographs of the lesions before administration of any pharmaceutical composition comprising vitamin A, and after daily administration of the composition for 14 days are shown for two different horses in FIGS. 1 and 2 (FIG. 1: horse ID 111112; FIG. 2: horse ID REG6).

FIG. 1 (a) (before any administration of the composition) shows a subtle core lesion in the lateral aspect of the superficial digital flexor tendon (SDFT) with generalised surrounding tendonitis. FIG. 1(b) (after daily administration of the composition for 14 days) shows that the core lesion has filled in somewhat and is less hypoechoic, suggesting that something has “plugged” the hole. Whilst the nature and quality of the tissue in the lesion is hard to assess with ultrasonography, it certainly appears to be making positive progress after only two weeks.

FIG. 2(a) (before any administration of the composition) shows a nasty SDFT core lesion in the medial aspect not quite involving paratenon. Again the lesion appears to be less hypoechoic on second scan (FIG. 2(b)—after daily administration of the composition for 14 days) suggesting the lesion is filling in with tissue of some sort. Again, the nature and quality of the tissue filling this lesion is hard to assess with ultrasound, but the lesion appears to be making positive progress after only two weeks.

### Conclusions

2 weeks in the field of equine chronic tendon/ligament injuries is a very short timescale and it is rare to see any significant change in these slow healing structures over such a short time period. In more acute injuries, there is a lot more early activity and ultrasonographic evidence of healing as the tendon responds to injury and the inflammatory cascade process commences.

The results presented here appear to show that vitamin A supplementation (by daily administration of the pharmaceutical composition) has had a positive effect on healing of the lesions after only 2 weeks.

### Example 7—Treatment of Equine Connective Tissue Injuries

This example describes the effect of a vitamin A supplement in treating connective tissue injuries in horses.

Connective tissue injuries are common in both human and equine athletes with massive physical, psychological and economic impact. These tendon and ligament injuries tend to take weeks to months of recovery time mainly consisting of rest and rehabilitation, depending on the severity. There are various outcomes possible following injury. Commonly, disorganised scar tissue is formed to replace the native tissue to quickly restore form at the expense of future function. This scar tissue is characterised by a disorganised extracellular matrix which does not have the same mechanical properties and integrity of the original tendon. It follows logically that these athletes are never quite able to achieve optimum performance once scarring has occurred and are also prone to re-injury. It is traditionally believed that once a scar has formed, it is there for life as it is believed to be essentially a passively maintained disorganised cluster of collagen, even after remodelling. However, there is recent evidence that suggests that scar tissue is actively maintained which may present an avenue to target established fibrotic tissue (Fear M et al. Changes in Fibroblast Phenotype and Matrix Turnover in Established Scar Tissue, J Burn Care Res 2019, Vol 40, Page 237).

Vitamin A has multiple functions in animals involving (and not limited to) development, and modulation of protein synthesis, and also possesses anti-inflammatory properties. There is some evidence that vitamin A plays a role in scar tissue formation and maintenance. This example aims to demonstrate the safety, and establish the clinical efficacy, of the usage of vitamin A supplementation in horses with tendon or ligament injuries.

### Methodology:

A prospective, single armed pilot trial of the efficacy and safety of Vitamin A supplementation in connective tissue injuries in horses was performed. The time since injury of each horse was noted. Full length ultrasonography of the injured tendon/ligament were performed at baseline as well as 3 weeks, 5 weeks and 7 weeks into the trial. Ultrasonography images were captured at the site of maximal injury. Depending on the nature of the injury, either cross-sectional or longitudinal views were taken. FIG. 3(a) shows a cross-sectional ultrasonogram of the major tendons/ligaments present in the left foot of a horse (normal equine anatomy). FIG. 3(b) shows a longitudinal ultrasonogram (left) of the left foot of a horse compared with a cross-sectional ultrasonogram (right) of the left foot of the same horse (normal equine anatomy). The lesions in the cross-sectional images were measured manually, aided by online software to determine the lesion size as a percentage of the overall cross-sectional area. The longitudinal images were presented to a consultant musculoskeletal radiologist who applied a 5-level grading system of the appearance of the injury corresponding to approximately 0%, 25%, 50%, 75% and 100% lesion size. Side effects and tolerability were also recorded.

### Vitamin A Supplement Used:

Vitamin A palmitate (retinyl palmitate) in a vehicle, delivered in dry feed based on the upper safe concentration in feeds (16,000 IU per kg feed dry matter).

### Administration of Vitamin A Supplement:

Horses with connective tissue injuries were orally administered vitamin A palmitate in a vehicle, at a dose of 160,000 IU vitamin A once per day for 7 weeks. The dose administered was decided based on the known toxic dose in horses (1,000 IU per kg (National Research Council. Nutrient Requirement of Horses: Fifth Revised Edition. The National Academies. 1989)) and the proposed upper safe concentration in feeds (16,000 IU per kg feed dry matter (Ralston S L. Nutritional Requirements of Horses and Other Equids. MSD Veterinary Manual, 2021)) which yielded a dose of 160,000 IU, corresponding to 32% of the toxic dose, assuming a 500 kg horse consuming 10 kg of dry feed. There were no adverse events reported and the supplement was well tolerated by the subjects.

### Subjects:

14 horses with 15 injured limbs were enrolled in the study between March and May 2021. The injuries comprised 9 tendon and 6 ligament injuries, with 12 injuries on the left side of the horse and 3 on the right. The mean time since injury was ˜ 12 months. The tendon injuries comprised 2 acute injuries (<1 month old) and 7 chronic injuries, with a mean time since injury of 13.1 months, and a range of 9-20 months. The ligamentous injuries comprised 6 chronic injuries, with a mean time since injury of 14.3 months and a range of 4-30 months.

The most commonly injured structure was the left fore superficial digital flexor tendon (n=8). The most commonly injured ligament was the left fore check ligament. The inclusion criteria were polo horses with diagnosed tendon/ligament injuries regardless of time since injury. There were no exclusion criteria.

### Statistical Analysis:

All statistical analysis was performed on SPSS v27. Missing values were handled by last observation carried forward. Descriptive statistics were used to describe baseline and follow-up values. The results were first tested for normality using the Shapiro-Wilk test. The results at weeks 3, 5 and 7 were then analysed compared to baseline using two-tailed paired Student's t-tests once normality was proven. One-tailed Pearson correlation test was performed to determine if outcomes was correlated with time since injury as we hypothesise that older, more established injuries may benefit less from our supplementation.

### Results

The mean lesion size was 41.44% at baseline, 35.87% at week 3, 28.37% at week 5 and 31.73% at week 7 (FIG. 4(a)). Shapiro-Wilk tests on the data revealed the lesion size was normally distributed at baseline and at weeks 3, 5 and 7. The calculated percentage improvement from baseline to week 7 was also deemed to be normally distributed. Thereafter, paired t-tests showed that the decrease from baseline to weeks 5 and 7 were statistically significant (p=<0.001 and 0.006 respectively) (FIG. 4(a)). When acute injury (<1 month) cases were removed (n=2), similar results were obtained with statistical significance being achieved at weeks 5 and 7 compared to baseline (p=<0.001 and p=0.017 respectively). FIGS. 5 to 16 show ultrasonograms of some of the tendon/ligamentous injuries of the data set at various time points from week 0. Table 1 shows the results of the trial, comprising data for each individual lesion.

One-tailed Pearson correlation coefficient on percentage improvement and time yielded a test statistic of r=−0.508 (p=0.027) indicating a statistically significant negative relationship (FIG. 4(b)). However, removal of 3 outliers yielded r=−0.806 (p=0.003).

TABLE 1
Full results table.

| | Time Since | | | | | Initial | Defect | Defect | Defect | |
|---|---|---|---|---|---|---|---|---|---|---|
| | Injury at | | Nature of | Site of | | Defect | Size at 3 | Size at 5 | Size at 7 | |
| ID | First Scan | | Injury | Injury | Ultrasound | Size (%) | weeks (%) | weeks (%) | weeks (%) | Comments |
| 1431 | >18 | months | Ligament | LF check | Cross- | 45.41 | 35.10 | 32.62 | 29.72 | Full work |
| | | | | | sectional | | | | | Movement artifact |
| 3465 | 9 | months | Tendon | LF SDFT | Longitudinal | 50.00 | 50.00 | 25.00 | 25.00 | Paddock rest |
| 8827 | >18 | months | Tendon | LF SDFT | Cross- | 62.30 | 46.03 | 36.40 | 53.79 | Low level |
| | | | | | sectional | | | | | exercise |
| | | | | | | | | | | Initial USS had |
| | | | | | | | | | | poor contact |
| | | | | | | | | | | 6 week f/u |
| | | | | | | | | | | movement artifact |
| 10520 | 6 | months | Ligament | LH medial | Cross- | 35.97 | 27.49 | 26.19 | 24.26 | Variation in |
| | | | | suspensory | sectional | | | | | quality and |
| | | | | branch | | | | | | positioning of |
| | | | | | | | | | | scans |
| 111112 | >12 | months | Tendon | LF SDFT | Cross- | 6.59 | 4.37 | 1.10 | 0.00 | Full work |
| | | | | | sectional | | | | | Tendon appears |
| | | | | | | | | | | normal, unable to |
| | | | | | | | | | | see discrete |
| | | | | | | | | | | lesion @ 6 weeks |
| | | | | | | | | | | but movement |
| | | | | | | | | | | artifact noted |
| 123345 | 9 | months | Tendon | LF SDFT | Cross- | 64.93 | 66.51 | 53.75 | 49.16 | Full work |
| | | | | | sectional | | | | | Painful clinically |
| 1234567 | 1 | month | Tendon | RF lateral | Cross- | 21.78 | 25.82 | 20.30 | 13.03 | Tendon splitting |
| | | | | SDFT | sectional | | | | | procedure |
| Q1Q | >12 | months | Tendon | LF SDFT | Cross- | 59.28 | 19.29 | 34.02 | 49.75 | Widespread |
| | | | | | sectional | | | | | scarring - |
| | | | | | | | | | | evolving in tissue |
| | | | | | | | | | | density |
| REG6 | 20 | months | Tendon | LF SDFT | Cross- | 60.00 | 54.63 | 41.84 | 47.21 | Horse continued |
| | | | | | sectional | | | | | exercising hence |
| | | | | | | | | | | worsening at |
| | | | | | | | | | | week 6 |
| REG7 | >12 | months | Tendon | LF SDFT | Longitudinal | 25.00 | 0.00 | 0.00 | 0.00 | |
| REG8 | 20 | months | Ligament | LF lateral | Longitudinal | 75.00 | 100.0 | 100.0 | 100.0 | |
| | | | | suspensory | | | | | | |
| | | | | branch | | | | | | |
| REG9 | Unknown | | Ligament | RF lateral | Cross- | 35.36 | 41.97 | 23.84 | 28.60 | |
| | | | | suspensory | sectional | | | | | |
| | | | | branch | | | | | | |
| 1833 | 4 | months | Ligament | LF check | Cross- | 20.11 | 20.11 | 9.17 | 9.17 | |
| | | | | | sectional | | | | | |
| 1833 | 30 | months | Ligament | RF check | Cross- | 40.00 | 40.00 | 39.54 | 39.54 | Only week 5 |
| | | | | | sectional | | | | | |
| 6168 | 1 | month | Tendon | LF lateral | Cross- | 19.81 | 6.79 | 6.79 | 6.79 | Only week 3 |
| | | | | SDFT | sectional | | | | | |

Note:
LF—left fore,
RF—right fore,
LH—left hind,
RH—right hind,
SDFT—superficial digital flexor tendon

### Discussion

The results of the study show that supplementation with vitamin A was extremely well tolerated with no signs of vitamin A toxicity noted in all subjects. Owing to the fact that the subjects were all specialised polo horses, there was a higher incidence of left sided injuries.

Our results show that it takes approximately 5 weeks for there to be significant improvements in the appearance of tendon/ligament injuries in horses which is maintained at least up to week 7 (FIG. 4(a)). Given that more than half of the injuries occurred over 12 months prior to the study and thus were very likely to be stable at the start of the trial, a noticeable improvement in 5 weeks is very astonishing. The results also show that there is a statistically significant linear negative correlation between the time between injury and starting vitamin A supplementation and outcomes (FIG. 4(b)), indicating that earlier vitamin A supplementation leads to better outcomes.

The dataset had three outliers as shown in scatter plot in FIG. 4(b): two at 12 months since injury, which both had 100% improvement, and one at 20 months since injury which had −25% improvement (i.e. the lesion got worse). The explanation for the outlier at 20 months since injury was that the ligamentous injury sustained by the subject was confirmed by a consultant radiologist to be a complete avulsion of the ligament. Short of surgery, there is no possibility of the ligament recovering following that type of injury, including with vitamin A supplement. The two outliers at 12 months since injury both had their lesions become unnoticeable by 7 weeks which is extremely promising.

General wound healing has 4 overlapping stages-haemostasis, inflammation, tissue formation and remodelling. Recent advancements in the field of wound healing suggest that the lack of inflammation in foetal wounds allows it to heal in a scarless manner, restoring the full function, flexibility and architecture of the native tissue (Galatz L M et al. Tendon Regeneration and Scar Formation: The Concept of Scarless Healing. J Orthop Res. 2015; 33:823-31). The known anti-inflammatory properties of vitamin A (Huang Z et al. Role of Vitamin A in the Immune System. J Clin Med. 2018; 7(9):258) support its use in the acute stages of tendon injuries, and indeed has been shown to increase the tensile strength of the healed tendon by double the control at day 45 in a 1990 study on chickens in Greenwald et al. Zone II Flexor Tendon Repair: Effects of Vitamins A, E, β-carotene. J Surg Res. 1990; 49(1):98-102, but does not explain the improvement found in the subjects of our study that have long-standing injuries.

The healing of tendon injuries starts with an early deposition of unoriented collagen fibres. Later on, a dynamic interplay of collagenolysis and deposition of oriented fibres determine the extent of restoration of normal tissue architecture (Greenwald et al., supra). Traditional thinking is that this process plateaus and leaves a permanent fibrotic scar. From our study, the fact that long-standing injuries (>12 months old) showed signs of improvement on ultrasonography is very encouraging especially with the recent suggestion that scar tissue is actively maintained. Fear et al. (supra) suggested this by demonstrating that fibroblasts in scar tissue are phenotypically different to fibroblasts in normal skin which is linked to the difference in matrix turnover.

The main cell type present in tendons are tenocytes (also known as tendon fibroblasts) and these maintain the tendon extracellular matrix ECM. Tendons are characterised by an exceptionally organised, anisotropic extracellular matrix with primarily type | collagen, although small amounts of type III collagen are also present (Fratzl P. Collagen: Structure and Mechanics, an Introduction. Collagen. Springer US; 2008. p. 1-13; Kannus P. Structure of the Tendon Connective Tissue. Scand J Med Sci Sport. 2000; 10(6):312-20). Equine tendon scar tissue has been shown to have higher than usual levels of type III collagen (20-30%) (Williams I et al. Cell Morphology and Collagen Types in Equine Tendon Scar. Res Vet

Sci. 1980; 28:302-10). Vitamin A is well known to play a role in the modulation of the synthesis of extracellular matrix proteins, including collagens, laminins, entactin, fibronectin, elastin and proteoglycans. It also has a role in the expression of various metalloproteinases, including collagenase. As scar tissue is due to excess deposition of disoriented collagen and physiologically abnormal proportions of collagen type by fibroblasts, this may allude to a plausible mechanism how vitamin A may influence fibroblasts maintaining scar tissue to instead produce native tendon tissue.

### Conclusion

In summary, our study found that vitamin A supplementation in horses with established tendon and ligament injuries led to radiological improvement and thus may positively influence the actively maintained characteristic extracellular matrix of scar tissue. We also found evidence that the degree of improvement with this supplementation is correlated with the time since injury. A potential mechanism by which it may act is upon fibroblasts or possibly their progenitor cells, mesenchymal stem cells. As scarring and fibrosis are seen in almost all areas of medicine, these findings have substantial implications and potential therapeutic uses if the mechanistic pathways are found to be more widely applicable.

### Example 8—Treatment of Equine Connective Tissue Injuries

This example describes the pathophysiology of equine tendon/ligament lesions before and after treatment with vitamin A supplement.

### Methodology:

Full length ultrasonography of the injured tendon/ligament was performed at the time of initial injury, baseline, as well as 3 weeks, 5 weeks and 7 weeks into the trial. Ultrasonography images were captured at the site of maximal injury. Depending on the nature of the injury, either cross-sectional or longitudinal views were taken. The lesions in these cross-sectional images were measured manually, aided by online software to determine the lesion size as a percentage of the overall cross-sectional area. The longitudinal images were presented to a consultant musculoskeletal radiologist who applied a 5-level grading system of the appearance of the injury corresponding to approximately 0%, 25%, 50%, 75% and 100% lesion size. Side effects and tolerability were also recorded.

The administration of vitamin A supplement, and the vitamin A supplement used, were the same as that of Example 7.

### Subjects:

7 horses with 7 injured limbs were enrolled in the study between March and May 2021. The injuries comprised 3 tendon and 4 ligament injuries. The inclusion criteria were polo horses with diagnosed tendon/ligament injuries regardless of time since injury. The exclusion criteria were horses with acute connective tissue injuries.

### Statistical Analysis:

All statistical analysis was performed on SPSS v27. Missing values were handled by last observation carried forward. Descriptive statistics were used to describe baseline and follow-up values. The results were first tested for normality using the Shapiro-Wilk test. The results at weeks 3, 5 and 7 were then analysed compared to baseline using two-tailed paired Student's t-tests once normality was proven.

### Results

FIG. 17(a) shows the size of the 7 lesions under investigation from the time of original injury, at baseline, and at 3, 5, and 7 weeks into the trial. The mean lesion size was 43.97% at the time of original injury and 49.43% at baseline (time treatment with vitamin A supplement began), but this difference was not statistically significant.

However, when tendon and ligament lesions were treated as two separate cohorts, there was an apparent divergence in natural healing progression between the two tissues from the time of original injury to when treatment began. As shown in FIG. 17(b), the tendons (n=3) tended to get worse as shown by an increase in lesion size across all three tendons, which was statistically significant (mean increase in lesion size=48.7%, p=0.03). Confirming the findings from previous analyses (Example 7), there was a statistically significant improvement in lesion size from baseline to week 7 with treatment with vitamin A supplement (mean improvement in lesion size=30.62% p=0.04).

As shown in FIG. 17(c), the ligaments tended to get better, demonstrated by a reduction in lesion size across three out of four lesions from the time of original injury to the start of treatment. The lesion that increased in size was an extremely severe injury, and the only subject across the entire study that did not improve even after treatment with vitamin A supplement. When analysing all 4 ligamentous injuries, there was no significant difference in lesion size at the time of original injury and the start of the trial (FIG. 17(c)). However, when excluding the known outlier, it was found that there was a statistically significant reduction in lesion size between the time of original injury and the start of the trial (FIG. 17(d)). There was also a reduction in the size of two of the three lesions of the data set from baseline to week 7 with treatment with vitamin A supplement (FIG. 17(d)). The lesion that stayed the same size from week 0 to week 7 was the oldest injury in the trial (30 months since original injury).

### Discussion

Interestingly, it appears that tendons and ligaments behave differently over their natural healing processes with ligamentous lesions either getting better or roughly staying the same from the time of original injury before improving with vitamin A supplement, and tendonous injuries getting significantly worse before improving with vitamin A supplement. These data suggest that there is a physiological difference between tendons and ligaments that results in diverging progression through wound healing, which provides guidance for the next steps.

### Example 9—Treatment of Equine Tendon Injury

This example shows the effect of vitamin A supplement on the echogenicity and health of tendon injury.

Structures composed of different tissue will have different echogenicities. The health of a tissue such as a tendon can be assessed by comparing the echogenicity of the tissue with that of a corresponding healthy tissue. Healthy tendons comprising native tissue and normal architecture are usually hyperechoic and appear white on the sonogram; they are capable of reflecting ultrasound that is cast over the tissue. An injured tendon comprising a lesion will appear as less hyperechoic, and more hypoechoic as tendon fibers are interrupted and defects are usually filled with fluid, blood, or fat. Severe lesions will be anechoic, and will display as completely dark on the sonogram.

### Methodology:

Full length ultrasonography of the injured tendon and adjacent healthy tendon was performed at baseline as well as 3 weeks, 5 weeks and 7 weeks into the trial. Ultrasonography images were captured at the site of maximal injury of the injured tendon. Depending on the nature of the injury, either cross-sectional or longitudinal views were taken. The echogenicity of a tendon lesion was assessed by comparing grey scale statistics of the tendon lesion with the values of healthy adjacent tendon tissue (FIG. 18(b)). Due to equine anatomy, there is always a directly adjacent healthy tendon to use as a control (ultrasonography is not very affected by depth of tissue). The statistics comprised determining the mean pixel intensity at a number of random points on the lesion and the healthy tendon, and calculating a mean value for the echogenicity of each tissue. A ratio of mean echnogenicity of the lesion and the healthy tissue was determined to assess the extent of damage/healing at the lesion.

Vitamin A supplement used, its administration, and the subjects investigated, comprise those of Example 7.

### Results

The mean echogenicity ratio of the lesion and healthy tendon increased each week from baseline to week 7 (FIG. 18(a)), which was statistically significant. A value of 1 would indicate perfect regeneration of native tendon. The mean echogenicity ratio was 0.52 at baseline, and 0.69 at week 7, equating to a % increase of 32.7%. As shown in FIG. 18(a), the mean echogenicity ratio from baseline to week 7 showed a continuing positive gradient.

This shows that the lesion is being replaced with native tendon tissue at a constant rate, providing evidence for continuing trials with full dose of vitamin A supplement past 7 weeks to achieve even more regeneration of the tendon. FIG. 18(b) shows an outline of the whole injured tendon and area of lesion. FIG. 18(c) shows an outline of an adjacent healthy tendon (as well as the outline of the injured tissue and lesion) used as a comparison tissue.

Interestingly, the injured tendon had lower echogenicity than the healthy tendon when the area of lesion was excluded and the remaining area of the injured tendon was examined (FIG. 19(b). FIG. 19(a) shows that the mean echogenicity ratio at week 0 was 0.76, which increased marginally to 0.82 at week 7, which was statistically significant. It was observed that the lesion size had normalised to the size of the tendon itself, suggesting that the lesion was not contained to the point of maximal injury, but that the health of the whole tendon was impaired.

### Example 10—Effect of Post-Treatment Maintenance Dose of Vitamin A Supplement on Connective Tissue Injuries

This example shows the effect of administering a post-treatment maintenance dose of vitamin A supplement on treating connective tissue injuries in horses. The results provide evidence for continuing at least a maintenance dose of vitamin A supplement past 7 weeks for connective tissue injury healing progression.

### Vitamin A Supplement Used:

Maintenance dose of vitamin A palmitate (retinyl palmitate) in a vehicle, delivered in dry feed at 8,000 IU per kg feed dry matter.

The methodology and subjects of the investigation comprise those of Example 7.

### Administration of Vitamin A Supplement:

At week 7 of full-treatment with vitamin A supplement, 7 horses with connective tissue injuries were orally administered vitamin A palmitate in a vehicle, at a dose of 80,000 IU vitamin A once per day for 7 weeks (half the treatment dose of vitamin A supplement). The known toxic dose in horses is 1,000 IU per kg (National Research Council. Nutrient Requirement of Horses: Fifth Revised Edition. The National Academies. 1989)). The dose of 80,000 IU corresponds to 16% of the toxic dose, assuming a 500 kg horse consuming 10 kg of dry feed. There were no adverse events reported and the supplement was well tolerated by the subjects. The remaining 7 horses were orally administered a placebo in a vehicle, delivered in dry feed. The trial was a double-blind randomised controlled trial.

### Results

The effect of post-treatment maintenance dose of vitamin A supplement on lesion size was investigated. FIG. 20(a) shows the mean lesion size from week 0 to week 7 on full-treatment doses of vitamin A supplement before splitting into the values of the maintenance dose and placebo groups. As described in Example 7, the calculated percentage improvement from baseline to week 7 was deemed to be normally distributed and paired t-tests showed that the decrease from baseline to weeks 5 and 7 were statistically significant (p=<0.001 and 0.006 respectively) (FIG. 4(a)). From week 7 to week 14, the mean lesion size of the placebo group increased from 31.73% to ˜35.50%. The mean lesion size of the maintenance dose group decreased from 31.73% to ˜20.00% from week 7 to week 14. The data shows that there was continued improvement in the healing of injured connective tissues for subjects that were administered maintenance dose of vitamin A supplement, and deterioration of the lesions for subjects that were administered placebo.

It was possible that the above results may give a slightly biased impression of the effect of the maintenance dose as the subjects in both groups (maintenance vs placebo) had different intragroup mean lesion sizes at week 7. FIG. 20(b) addresses this issue by showing the progression of both groups as well as the average value from Week 0 through to Week 14. Both groups continued to improve from week 7, however the figure still shows a more dramatic improvement in subjects taking a maintenance dose compared to those on placebo (−24.5% vs-6.28%).

One subject became re-injured after treatment with full-treatment doses of vitamin A supplement stopped and a maintenance dose of vitamin A supplement was administered for 7 weeks. FIG. 21 shows an ultrasonogram at baseline (FIG. 21(a)), week 7 (7 weeks of treatment with full-treatment doses of vitamin A supplement) (FIG. 21(b)), and week 14 (7 weeks of treatment with maintenance dose vitamin A supplement) (FIG. 21(c)). The size of the lesion decreases at 7 weeks of full-treatment doses, as shown by a reduction in the hypoechoic area of the ultrasonogram (FIG. 21(b)). However, the lesion becomes much more hypoechoic after post-treatment maintenance dose has been administered for 7 weeks (FIG. 21(c)). Thus, the health of this particular lesion deteriorated after treatment with maintenance dose of vitamin A supplement. This result is different to those described above, wherein the maintenance dose cohort showed improvement in the regeneration of the lesion. The re-injury presented in FIG. 21 provides evidence that, in some cases, treatment with full-treatment doses of vitamin A may be required for longer periods for regeneration of the tissue.

### Example 11—Effect of Treatment and Activity Level on Connective Tissue Improvement

This example shows the effect of activity level on connective tissue improvement in horses on vitamin A supplement.

The vitamin A supplement used, its administration, and the subjects of the investigation comprise those of Example 7.

### Methodology

Improvement (as a % of baseline) of the injured tendon/ligament was performed at 7 weeks into the trial. All 14 horses were administered full-treatment doses of vitamin A supplement from week 0 to week 7 whilst being put to work. 7 horses were put on light/no work and 7 on full work. The allocation of each horse into one of the two groups was determined by the respective owner of the horse, who decided based on whether they could afford for the horse to be put on light work (and thus investigate the isolated effect of vitamin A supplement on lesion progression) or whether the horse was required to be put back to full work.

### Results

FIG. 22 shows the effect of activity level on the healing of connective tissue injury. The graph shows that the horses put on light or no work (n=7) demonstrated less of an improvement in the health of the tendon/lesion (30.35% improvement) than the horses put on full work (45.05% improvement) (n=7). The results suggest that the effect of vitamin A supplement on connective tissue injury is enhanced by increasing activity. However, increasing activity too much is likely to result in a decrease in % improvement and worsening of the lesion (not shown in this example).

### Example 12—Pharmaceutical Composition for Oral Administration

A pharmaceutical composition for oral administration to an adult human comprises a single unit dose of 50,000 IU Vitamin A in coconut oil. The pharmaceutical composition is provided in a sterile sachet.

### Example 13—Pharmaceutical Composition for Oral Administration

A pharmaceutical composition for oral administration to an adult human comprises a single unit dose of 25,000 IU/mL Vitamin A in coconut oil. The pharmaceutical composition is provided in a sterile sachet.

1. Vitamin A for use in inhibition of scar tissue formation in a subject.

2. Use of vitamin A in the manufacture of a medicament for the inhibition of scar tissue formation in a subject.

3. Vitamin A for use in the treatment of tissue damage in a subject.

4. Use of vitamin A in the manufacture of a medicament for the treatment of tissue damage in a subject.

5. Vitamin A for use according to claim 1 or 3, or use of vitamin A according to claim 2 or 4, wherein the vitamin A comprises isolated vitamin A.

6. Vitamin A for use, or use of vitamin A, according to any preceding claim, wherein the vitamin A comprises a preformed vitamin A, such as a retinyl ester or retinol.

7. Vitamin A for use, or use of vitamin A, according to any preceding claim wherein the vitamin A comprises a provitamin A, such as a carotenoid.

8. Vitamin A for use, or use of vitamin A, according to any preceding claim wherein the vitamin A comprises a bioactive form of vitamin A, such as retinal or retinoic acid.

9. Vitamin A for use, or use of vitamin A, according to any preceding claim for administration at a dose in excess of a Tolerable Upper Limit Intake Level (UL) for the subject.

10. Vitamin A for use, or use of vitamin A, according to any preceding claim wherein the subject is a human subject.

11. Vitamin A for use, or use of vitamin A, according to claim 10 for administration to the subject at a dose of >10,000 to 100,000 IU vitamin A per day.

12. Vitamin A for use, or use of vitamin A, according to claim 11 for administration to the subject at a dose of about 25,000-100,000, 50,000-100,000, or 75,000-100,000 IU vitamin A per day.

13. Vitamin A for use, or use of vitamin A, according to any preceding claim for administration to the subject once per day.

14. Vitamin A for use, or use of vitamin A, according to claim 13 for administration for at least 3 days from the day of first administration to the subject.

15. Vitamin A for use, or use of vitamin A, according to claim 13 for administration for at least a week, at least a month, or at least 6 months from the day of first administration to the subject.

16. Vitamin A for use, or use of vitamin A, according to any of claims 13 to 15 for administration to the subject for up to 6 years from the day of first administration to the subject.

17. Vitamin A for use, or use of vitamin A, according to any preceding claim for administration systemically.

18. Vitamin A for use, or use of vitamin A, according to claim 17 for administration to the subject orally or intravenously.

19. Vitamin A for use, or use of vitamin A, according to any preceding claim for inhibition of scar tissue formation in the subject following an injury to the subject.

20. Vitamin A for use, or use of vitamin A, according to any of claims 1 to 18 for the treatment of tissue damage in the subject following an injury to the subject.

21. Vitamin A for use, or use of vitamin A, according to claim 19 or 20 for the treatment of the injury.

22. Vitamin A for use, or use of vitamin A, according to any of claims 19 to 21 wherein the injury comprises a traumatic injury.

23. Vitamin A for use, or use of vitamin A, according to any of claims 19 to 22 wherein the injury comprises a neurological injury, such as a spinal cord injury, a brain injury, or a peripheral nerve injury.

24. Vitamin A for use, or use of vitamin A, according to claim 23 wherein the inhibition of scar tissue formation comprises inhibition of glial scar tissue formation.

25. Vitamin A for use, or use of vitamin A, according to any of claims 19 to 22 wherein the injury comprises a soft tissue injury, such as a tendon injury or a ligament injury.

26. Vitamin A for use, or use of vitamin A, according to any preceding claim for administration to the subject prior to, with, or subsequent to administration of one or more regenerative cells.

27. Vitamin A for use, or use of vitamin A, according to claim 26 for administration to the subject prior to an administration of the one or more regenerative cells.

28. Vitamin A for use, or use of vitamin A, according to claim 26 or 27 wherein the administration of vitamin A is within 48 hours of the administration of the one or more regenerative cells.

29. Vitamin A for use, or use of vitamin A, according to any preceding claim, wherein the subject has been administered one or more regenerative cells.

30. Vitamin A for use, or use of vitamin A, according to any of claims 26 to 29 wherein the one or more regenerative cells are autologous to the subject.

31. Vitamin A for use, or use of vitamin A, according to any of claims 26 to 30 wherein the one or more regenerative cells comprise one or more stem cells.

32. Vitamin A for use, or use of vitamin A, according to claim 31 wherein the one or more stem cells comprises one or more pluripotent stem cells, such as one or more embryonic stem cells or induced pluripotent stem cells.

33. Vitamin A for use, or use of vitamin A, according to any of claims 26 to 32 wherein the one or more stem cells comprise one or more multipotent stem cells.

34. Vitamin A for use, or use of vitamin A, according to claim 33 wherein the one or more multipotent stem cells comprise one or more neural stem cells.

35. Vitamin A for use, or use of vitamin A, according to any of claims 26 to 34 for the treatment of a neurological injury, such as a spinal cord injury, a brain injury, or a peripheral nerve injury.

36. Vitamin A for use, or use of vitamin A, according to claim 33 wherein the one or more multipotent stem cells comprise one or more mesenchymal stem cells, adipose-derived stem cells, or tendon-derived stem cells.

37. Vitamin A for use, or use of vitamin A, according to any of claim 26 to 33, or 36 for the treatment of a soft tissue injury, such as a tendon injury or a ligament injury.

38. Vitamin A for use, or use of vitamin A, according to any of claims 26 to 37 wherein the one or more regenerative cells are administered to a site that is local to the site of an injury to the subject.

39. Vitamin A for use, or use of vitamin A, according to any preceding claim, wherein the vitamin A is the only non-cellular, non-antibiotic, active agent to be administered to the subject.

40. A multiple-dose formulation which comprises a plurality of separate unit doses of vitamin A, wherein each unit dose comprises >10,000 IU to 100,000 IU vitamin A.

41. A multiple-dose formulation according to claim 40 wherein each unit dose comprises about 25,000-100,000, 50,000-100,000, or 75,000-100,000 IU vitamin A.

42. A multiple-dose formulation according to claim 40 or 41 wherein the vitamin A comprises isolated vitamin A.

43. A multiple-dose formulation according to any of claims 40 to 42 wherein the vitamin A comprises a preformed vitamin A, such as a retinyl ester or retinol.

44. A multiple-dose formulation according to any of claims 40 to 43 wherein the vitamin A comprises a provitamin A, such as a carotenoid.

45. A multiple-dose formulation according to any of claims 40 to 44 wherein the vitamin A comprises a bioactive form of vitamin A, such as retinal or retinoic acid.

46. A multiple-dose formulation according to any of claims 40 to 45 wherein the vitamin A is part of a pharmaceutical composition comprising vitamin A and a pharmaceutically acceptable carrier, excipient or diluent.

47. A multiple-dose formulation according to claim 46 wherein vitamin A is the only non-cellular, non-antibiotic, active agent present in the pharmaceutical composition.

48. A multiple-dose formulation according to any of claims 40 to 47 comprising at least 7, at least 30, or at least 100 separate unit doses of vitamin A.

49. A multiple-dose formulation according to any of claims 40 to 48 for use as a medicament.

50. A multiple-dose formulation according to any of claims 40 to 48 for use in inhibition of scar tissue formation in a subject.

51. Use of a multiple-dose formulation according to any of claims 40 to 48 in the manufacture of a medicament for inhibition of scar tissue formation in a subject.

52. A multiple-dose formulation according to any of claims 40 to 48 for use in the treatment of tissue damage in a subject.

53. Use of a multiple-dose formulation according to any of claims 40 to 48 in the manufacture of a medicament for the treatment of tissue damage in a subject.

54. A multiple-dose formulation according to any of claim 40 to 48, or 50 for use in the treatment of an injury to the subject.

55. Use of a multiple-dose formulation according to any of claim 40 to 48, or 50 in the manufacture of a medicament for the treatment of an injury to the subject.

56. A combined preparation comprising: (a) vitamin A and; (b) one or more regenerative cells.

57. A combined preparation according to claim 56 wherein the vitamin A comprises isolated vitamin A.

58. A combined preparation according to claim 56 or 57 wherein the vitamin A comprises a preformed vitamin A, such as a retinyl ester or retinol.

59. A combined preparation according to any of claims 56 to 58 wherein the vitamin A comprises a provitamin A, such as a carotenoid.

60. A combined preparation according to any of claims 56 to 59 wherein the vitamin A comprises a bioactive form of vitamin A, such as retinal or retinoic acid.

61. A combined preparation according to any of claims 56 to 60 wherein the vitamin A is part of a pharmaceutical composition comprising vitamin A and a pharmaceutically acceptable carrier, excipient or diluent.

62. A combined preparation according to claim 61, wherein vitamin A is the only non-cellular, non-antibiotic, active agent present in the pharmaceutical composition.

63. A combined preparation according to any of claims 56 to 62 wherein the one or more regenerative cells are part of a pharmaceutical composition comprising the one or more regenerative cells and a pharmaceutically acceptable carrier, excipient or diluent.

64. A combined preparation according to any of claims 56 to 63 wherein the vitamin A comprises one or more separate unit doses, wherein the or each unit dose comprises >10,000 to 100,000 IU vitamin A.

65. A combined preparation according to claim 64 wherein the or each unit dose comprises about 25,000-100,000, 50,000-100,000, or 75,000-100,000 IU vitamin A.

66. A combined preparation according to any of claims 56 to 65 wherein the one or more regenerative cells comprise one or more stem cells.

67. A combined preparation according to claim 66 wherein the one or more stem cells comprises one or more pluripotent stem cells, such as one or more embryonic stem cells or induced pluripotent stem cells.

68. A combined preparation according to claim 66 wherein the one or more stem cells comprise one or more multipotent stem cells.

69. A combined preparation according to claim 68 wherein the one or more multipotent stem cells comprise one or more neural stem cells.

70. A combined preparation according to claim 68 wherein the one or more multipotent stem cells comprise one or more mesenchymal stem cells, adipose-derived stem cells, or tendon-derived stem cells.

71. A combined preparation according to any of claims 56 to 70 for use as a medicament.

72. A combined preparation according to any of claims 56 to 70 for use in inhibition of scar tissue formation in a subject.

73. Use of a combined preparation according to any of claims 56 to 70 in the manufacture of a medicament for the treatment of scar tissue formation in a subject.

74. A combined preparation according to any of claims 56 to 70 for use in the treatment of tissue damage in a subject.

75. Use of a combined preparation according to any of claims 56 to 70 in the manufacture of a medicament for the treatment of tissue damage in a subject.

76. A combined preparation according to any of claims 56 to 70 for use in the treatment of an injury to the subject.

77. Use of a combined preparation according to any of claims 56 to 70 in the manufacture of a medicament for the treatment of an injury to the subject.

78. A method of inhibiting scar tissue formation in a subject comprising administering to the subject an effective amount of vitamin A.

79. A method of treating tissue damage in a subject comprising administering to the subject an effective amount of vitamin A.

80. A method of treating an injury to a subject comprising administering to the subject an effective amount of vitamin A.

81. A method according to any of claims 78 to 80, wherein the vitamin A comprises isolated vitamin A.

82. A method according to any of claims 78 to 81 wherein the vitamin A comprises a preformed vitamin A, such as a retinyl ester or retinol.

83. A method according to any of claims 78 to 82 wherein the vitamin A comprises a provitamin A, such as a carotenoid.

84. A method according to any of claims 78 to 83 wherein the vitamin A comprises a bioactive form of vitamin A, such as retinal or retinoic acid.

85. A method according to any of claims 78 to 84 wherein the vitamin A is administered at a dose in excess of a Tolerable Upper Limit Intake Level (UL) for the subject.

86. A method according to any of claims 78 to 85 wherein the subject is a human subject.

87. A method according to claim 86 wherein the vitamin A is administered to the subject at a dose of >10,000 to 100,000 IU vitamin A per day.

88. A method according to claim 87 wherein the vitamin A is administered to the subject at a dose of about 25,000-100,000, 50,000-100,000, or 75,000-100,000 IU vitamin A per day.

89. A method according to any of claims 78 to 88 wherein the vitamin A is administered to the subject once per day.

90. A method according to claim 89 wherein the vitamin A is administered to the subject once per day for at least 3 days from the day of first administration to the subject.

91. A method according to claim 89 wherein the vitamin A is administered to the subject once per day for at least a week, at least a month, or at least 6 months from the day of first administration to the subject.

92. A method according to any of claims 89 to 91 wherein the vitamin A is administered to the subject for up to 6 years from the day of first administration to the subject.

93. A method according to any of claims 78 to 92 wherein the vitamin A is administered systemically to the subject.

94. A method according to claim 93 wherein the vitamin A is administered orally or intravenously to the subject.

95. A method according to any of claims 78 to 94 for the treatment of an injury to the subject wherein the vitamin A is administered following the injury to the subject.

96. A method according to claim 95, wherein the vitamin A is first administered to the subject within a week of the injury.

97. A method according to claim 95 or 96 wherein the injury comprises a traumatic injury.

98. A method according to any of claims 95 to 97 wherein the injury comprises a neurological injury, such as a spinal cord injury, a brain injury, or a peripheral nerve injury.

99. A method according to claim 98 wherein administration of the vitamin A inhibits glial scar tissue formation.

100. A method according to any of claims 95 to 97 wherein the injury comprises a soft tissue injury, such as a tendon injury or a ligament injury.

101. A method according to any of claims 78 to 100 wherein the vitamin A is administered to the subject prior to, with, or subsequent to administration of one or more regenerative cells.

102. A method according to claim 101 wherein the vitamin A is administered to the subject prior to an administration of the one or more regenerative cells.

103. A method according to claim 101 or 102 wherein the vitamin A is administered within 48 hours of the administration of the one or more regenerative cells.

104. A method according to any of claims 101 to 103 wherein the one or more regenerative cells are autologous to the subject.

105. A method according to any of claims 101 to 104 wherein the one or more regenerative cells comprise one or more stem cells.

106. A method according to any of claims 101 to 105 wherein the one or more stem cells comprise one or more pluripotent stem cells, such as one or more embryonic stem cells or induced pluripotent stem cells.

107. A method according to any of claims 101 to 106 wherein the one or more stem cells comprise one or more multipotent stem cells.

108. A method according to claim 107 wherein the one or more multipotent stem cells comprise one or more neural stem cells.

109. A method according to any of claims 101 to 108 for the treatment of a neurological injury, such as a spinal cord injury, a brain injury, or a peripheral nerve injury.

110. A method according to claim 107 wherein the one or more multipotent stem cells comprise one or more mesenchymal stem cells, adipose-derived stem cells, or tendon-derived stem cells.

111. A method according to any of claim 101 to 107, or 110 for the treatment of a soft tissue injury, such as a tendon injury or a ligament injury.

112. A method according to any of claims 101 to 111 wherein the one or more regenerative cells are administered to a site that is local to the site of an injury to the subject.

113. A method according to any of claims 78 to 103, wherein the vitamin A is the only non-cellular, non-antibiotic, active agent to be administered to the subject.

114. A method according to any of claims 78 to 113 wherein the subject is a human subject.

115. A method according to any of claims 78 to 113 wherein the subject is a human subject who is at least 30 years old.

116. A method according to any of claims 78 to 113 wherein the subject is a non-human subject, such as a horse or a dog.

117. Use according to claim 25 or 37, or a method according to claim 100 or 111, wherein the subject is a horse.

118. Use according to claim 23 or 35, or a method according to claim 98 or 109, wherein the subject is a dog.

119. Vitamin A for use, or use of vitamin A, according to any of claims 1 to 39, wherein the vitamin A inhibits scar tissue formation by anti-inflammatory action.

120. Vitamin A for use, or use of vitamin A, according to any of claim 1 to 39, or 119, wherein the vitamin A inhibits scar tissue formation by increasing expression of collagenase.

121. Vitamin A for use, or use of vitamin A, according to any of claim 1 to 39, 119, or 120, wherein the scar tissue is actively maintained scar tissue.

122. Vitamin A for use, or use of vitamin A, according to any of claims 1 to 39, or 119 to 121, for administration for at least five weeks from the day of first administration to the subject.

123. Vitamin A for use, or use of vitamin A, according to any of claims 1 to 39, or 119 to 122, for administration to the subject at a dose upto 50% of a minimum toxic dose for the subject.

124. Vitamin A for use, or use of vitamin A, according to any of claims 1 to 39, or 119 to 123 for administration to the subject at a dose of at least 5% of a minimum toxic dose for the subject.

125. A method according to any of claims 78 to 116, wherein the vitamin A is administered for at least five weeks from the day of first administration to the subject.

126. A method according to any of claim 78 to 116, or 125, wherein the vitamin A is administered to the subject at a dose upto 50% of a minimum toxic dose for the subject.

127. A method according to any of claim 78 to 116, 125, or 126, wherein the vitamin A is administered to the subject at a dose of at least 5% of a minimum toxic dose for the subject.

128. A method according to any of claims 78 to 116, or 125 to 127, wherein the vitamin A inhibits scar tissue formation by anti-inflammatory action.

129. A method according to any of claims 78 to 116, or 125 to 128, wherein the vitamin A inhibits scar tissue formation by increasing expression of collagenase.

130. A method according to any of claims 78 to 116, or 125 to 129, wherein the scar tissue is actively maintained scar tissue.

131. A method according to claim 116, or use or a method according to claim 117, wherein the horse is administered a dose of 25,000-250,000, 50,000-250,000, 75,000-250,000, 100,000-250,000, 125,000-250,000, 150,000-250,000, 175,000-250,000, or 200,000-250,000 IU vitamin A per day.

132. A method according to claim 116, or use or a method according to claim 117, wherein the horse is administered a dose of 25,000-50,000, 25,000-75,000, 25,000-100,000, 25,000-125,000, 25,000-150,000, 25,000-175,000, or 25,000-200,000 IU vitamin A per day.

133. A multiple-dose formulation which comprises a plurality of separate unit doses of vitamin A, wherein each unit dose comprises 25,000-250,000, 50,000-250,000, 75,000-250,000, 100,000-250,000, 125,000-250,000, 150,000-250,000, 175,000-250,000, or 200,000-250,000 IU vitamin A.

134. A multiple-dose formulation which comprises a plurality of separate unit doses of vitamin A, wherein each unit dose comprises 25,000-50,000, 25,000-75,000, 25,000-100,000, 25,000-125,000, 25,000-150,000, 25,000-175,000, or 25,000-200,000 IU vitamin A.

135. A multiple-dose formulation according to claim 133 or 134 for administration to a horse.

136. Vitamin A for use, or use of vitamin A, according to any of claims 1 to 39, or 119 to 124, for administration to the subject at a maintenance dose, wherein the maintenance dose is less than a full treatment dose.

137. Vitamin A for use, or use of vitamin A, according to claim 136, wherein the maintenance dose is up to three quarters of a full treatment dose.

138. Vitamin A for use, or use of vitamin A, according to claim 136 or 137, wherein the maintenance dose is up to two-thirds of a full treatment dose.

139. Vitamin A for use, or use of vitamin A, according to any of claims 136 to 138, wherein the maintenance dose is at least a quarter of a full treatment dose.

140. Vitamin A for use, or use of vitamin A, according to any of claims 136 to 139, wherein the maintenance dose is to be administered after the subject has been administered one or more full-treatment doses.

141. Vitamin A for use, or use of vitamin A, according to any of claims 136 to 140, wherein the maintenance dose is to be administered from the day after the last administration of a full-treatment dose to the subject.

142. Vitamin A for use, or use of vitamin A, according to any of claims 136 to 141, wherein a plurality of maintenance doses is to be administered.

143. Vitamin A for use, or use of vitamin A, according to claim 142, wherein the maintenance doses are to be administered for at least four weeks from the day of first administration of a maintenance dose to the subject.

144. Vitamin A for use, or use of vitamin A, according to claim 143, wherein the maintenance doses are to be administered for at least 12 weeks from the day of first administration of a maintenance dose to the subject.

145. Vitamin A for use, or use of vitamin A, according to claim 143, wherein the maintenance doses are to be administered for at least 6 months from the day of first administration of a maintenance dose to the subject.

146. Vitamin A for use, or use of vitamin A, according to claim 143, wherein the maintenance doses are to be administered for at least 12 months from the day of first administration of a maintenance dose to the subject.

147. Vitamin A for use, or use of vitamin A, according to any of claims 142 to 146, wherein the maintenance doses are to be administered for up to 6 years from the day of first administration of a maintenance dose to the subject.

148. Vitamin A for use, or use of vitamin A, according to any of claims 136 to 147, wherein the subject is a human subject.

149. Vitamin A for use, or use of vitamin A, according to claim 148, wherein the, or each maintenance dose comprises >2,500 IU to 75,000 IU vitamin A per day.

150. Vitamin A for use, or use of vitamin A, according to claim 148 or 149, wherein the, or each maintenance dose comprises 5,000-75,000, 10,000-75,000, or 20,000-75,000 IU vitamin A per day.

151. Vitamin A for use, or use of vitamin A, according to claim 148 or 149, wherein the, or each maintenance dose comprises 5,000-50,000, 10,000-50,000, or 20,000-50,000 IU vitamin A per day.

152. Vitamin A for use, or use of vitamin A, according to any of claims 136 to 147, wherein the subject is a non-human subject.

153. Vitamin A for use, or use of vitamin A, according to claim 152, wherein the subject is a horse.

154. Vitamin A for use, or use of vitamin A, according to claim 153, wherein the, or each maintenance dose comprises 10,000-200,000, 20,000-150,000, 40,000-100,000, or 60,000-100,000 IU vitamin A per day.

155. A multiple-dose formulation which comprises a plurality of separate unit doses of vitamin A, wherein each unit dose is a maintenance dose of vitamin A, and wherein each unit dose comprises >2,500 IU to 75,000 IU vitamin A.

156. A multiple-dose formulation according to claim 155, wherein the, or each unit dose comprises 5,000-75,000, 10,000-75,000, or 20,000-75,000 IU vitamin A.

157. A multiple-dose formulation according to claim 155 or 156, wherein the, or each unit dose comprises 5,000-50,000, 10,000-50,000, or 20,000-50,000 IU vitamin A.

158. A multiple-dose formulation according to any of claims 155 to 157 for administration to a human subject.

159. A multiple-dose formulation which comprises a plurality of separate unit doses of vitamin A, wherein each unit dose is a maintenance dose of vitamin A, and wherein each unit dose comprises 10,000-200,000, 20,000-150,000, 40,000-100,000, or 60,000-100,000 IU vitamin A.

160. A multiple-dose formulation according to claim 159 for administration to a horse.

161. A multiple-dose formulation according to any of claims 155 to 160, wherein the vitamin A comprises isolated vitamin A.

162. A multiple-dose formulation according to any of claims 155 to 161, wherein the vitamin A comprises a preformed vitamin A, such as a retinyl ester or retinol.

163. A multiple-dose formulation according to any of claims 155 to 162, wherein the vitamin A comprises a provitamin A, such as a carotenoid.

164. A multiple-dose formulation according to any of claims 155 to 163, wherein the vitamin A comprises a bioactive form of vitamin A, such as retinal or retinoic acid.

165. A multiple-dose formulation according to any of claims 155 to 164, wherein the vitamin A is part of a pharmaceutical composition comprising vitamin A and a pharmaceutically acceptable carrier, excipient or diluent.

166. A multiple-dose formulation according to claim 165, wherein vitamin A is the only non-cellular, non-antibiotic, active agent present in the pharmaceutical composition.

167. A multiple-dose formulation according to any of claims 155 to 166 comprising at least 7, at least 30, or at least 100 separate unit doses of vitamin A.

168. A multiple-dose formulation which comprises: a first plurality of separate unit doses of vitamin A, wherein each unit dose of the first plurality of separate unit doses is a full treatment unit dose of vitamin A as recited in any of claims 40 to 48; and a second plurality of separate unit doses of vitamin A, wherein each unit dose of the second plurality of separate unit doses is a maintenance dose of vitamin A as recited in any of claims 155 to 158, or 161 to 167.

169. A multiple-dose formulation which comprises: a first plurality of separate unit doses of vitamin A, wherein each unit dose of the first plurality of separate unit doses is a full treatment unit dose of vitamin A as recited in any of claims 133 to 135; and a second plurality of separate unit doses of vitamin A, wherein each unit dose of the second plurality of separate unit doses is a maintenance dose of vitamin A as recited in claim 159 or 160.

170. A multiple-dose formulation according to any of claims 155 to 169 for use as a medicament.

171. A multiple-dose formulation according to any of claims 155 to 169 for use in inhibition of scar tissue formation in a subject.

172. Use of a multiple-dose formulation according to any of claims 155 to 169 in the manufacture of a medicament for inhibition of scar tissue formation in a subject.

173. A multiple-dose formulation according to any of claims 155 to 169 for use in the treatment of tissue damage in a subject.

174. Use of a multiple-dose formulation according to any of claims 155 to 169 in the manufacture of a medicament for the treatment of tissue damage in a subject.

175. A multiple-dose formulation according to any of claims 155 to 169 for use in the treatment of an injury to the subject.

176. Use of a multiple-dose formulation according to any of claims 155 to 169 in the manufacture of a medicament for the treatment of an injury to the subject.

177. A multiple-dose formulation according to claim 175, or use according to claim 176, wherein the injury comprises a soft tissue injury, such as a tendon injury or a ligament injury.

178. A method according to any of claims 78 to 116, or 125 to 132, wherein the subject is administered a maintenance dose of vitamin A, wherein the maintenance dose is less than a full treatment dose.

179. A method according to claim 178, wherein the maintenance dose is up to three quarters of a full treatment dose.

180. A method according to claim 178 or 179, wherein the maintenance dose is up to two-thirds of a full treatment dose.

181. A method according to any of claims 178 to 180, wherein the maintenance dose is at least a quarter of a full treatment dose.

182. A method according to any of claims 178 to 181, wherein the maintenance dose is administered after the subject has been administered one or more full-treatment doses.

183. A method according to any of claims 178 to 182, wherein the maintenance dose is administered from the day after the last administration of a full-treatment dose to the subject.

184. A method according to any of claims 178 to 183, wherein a plurality of maintenance doses administered to the subject.

185. A method according to claim 184, wherein the maintenance doses are administered for at least four weeks from the day of first administration of a maintenance dose to the subject.

186. A method according to claim 185, wherein the maintenance doses are administered for at least 12 weeks from the day of first administration of a maintenance dose to the subject.

187. A method according to claim 185, wherein the maintenance doses are administered for at least 6 months from the day of first administration of a maintenance dose to the subject.

188. A method according to claim 185, wherein the maintenance doses are administered for at least 12 months from the day of first administration of a maintenance dose to the subject.

189. A method according to any of claims to 184 to 188, wherein the maintenance doses are administered for up to 6 years from the day of first administration of a maintenance dose to the subject.

190. A method according to any of claims to 184 to 189, wherein the subject is a human subject.

191. A method according to claim 190, wherein the, or each maintenance dose comprises >2,500 IU to 75,000 IU vitamin A per day.

192. A method according to claim 190 or 191, wherein the, or each maintenance dose comprises 5,000-75,000, 10,000-75,000, or 20,000-75,000 IU vitamin A per day.

193. A method according to claim 190 or 191, wherein the, or each maintenance dose comprises 5,000-50,000, 10,000-50,000, or 20,000-50,000 IU vitamin A per day.

194. A method according to any of claims 184 to 189, wherein the subject is a non-human subject.

195. A method according to claim 194, wherein the subject is a horse.

196. A method according to claim 195, wherein the, or each maintenance dose comprises 10,000-200,000, 20,000-150,000, 40,000-100,000, or 60,000-100,000 IU vitamin A per day.
